(19) United States
(12) Patent Application Publication
(10) Pub. No.: US 2026/0240993 A1
(43) Pub. Date: Aug. 20, 2026

(54) PARAINFLUENZA VIRUS 3 MONOCLONAL ANTIBODIES

(71) Applicant: Florida State University Research Foundation, Inc., Tallahassee, FL (US)

(72) Inventors: Jarrod J. Mousa, Tallahassee, FL (US); Katelyn D. McCaffrey, Tallahassee, FL (US); Behrouz Ghazi Esfahani, Tallahassee, FL (US)

(21) Appl. No.: 19/390,966

(22) Filed: Nov. 17, 2025

Related U.S. Application Data
(60) Provisional application No. 63/721,628, filed on Nov. 18, 2024.

(51) Int. Cl. A61K 39/42; A61K 38/00; A61K 39/00; A61K 39/155; A61P 31/14; C07K 16/11; C12Q 1/70

(52) U.S. Cl. CPC A61K 39/42; A61K 38/00; A61K 39/155; A61P 31/14; C07K 16/11; C12Q 1/70; A61K 2039/543; C07K 2317/21; C07K 2317/31; C07K 2317/732; C07K 2317/734; C07K 2317/77; C07K 2317/94; C12Q 2600/112

(57) ABSTRACT

In one aspect, the disclosure relates to isolated human antibodies that bind to at least one protein from a parainfluenza virus (PIV), including, but not limited to, PIV3, pharmaceutical compositions comprising same, methods of treating or preventing PIV infection using same, and methods for detecting PIV in a sample from a subject using same. This abstract is intended as a scanning tool for purposes of searching in the particular art and is not intended to be limiting of the present disclosure.

### CROSS-REFERENCE TO RELATED APPLICATIONS

This application claims the benefit of U.S. Provisional Application No. 63/721,628 filed on Nov. 18, 2024, which is incorporated herein by reference in its entirety.

### STATEMENT REGARDING FEDERALLY SPONSORED RESEARCH OR DEVELOPMENT

This invention was made with government support under grant numbers R01AI143865 and R56AI181850 awarded by the National Institutes of Health. The government has certain rights in the invention.

### CROSS REFERENCE TO SEQUENCE LISTING

This application contains a sequence listing filed in ST.26 format entitled “930603-1510_Sequence_Listing.xml” created on Nov. 17, 2025, and having a size of 153,855 bytes, is incorporated by reference in its entirety.

### BACKGROUND

Human parainfluenza viruses (PIVs) are clinically prevalent viruses consisting of four types circulating globally each year. Although viral isolates have been detected in people of all ages, PIVs primarily cause severe disease in infants and young children, the elderly, the immunocompromised, and those with preexisting comorbidities, such as lung transplants or asthma. With clinical manifestations ranging from mild upper respiratory distress to bronchitis and pneumonia, PIV infection can lead to hospitalization and death in these high-risk populations. PIV type 3 (PIV3) is the second leading cause of bronchitis and pneumonia in children after respiratory syncytial virus (RSV). Enhanced respiratory disease caused by PIV3 infection typically afflicts young children during their first 2 years of life. Nearly 60% of children show serological evidence for PIV3 infection before the age of 5, with reinfection occurring throughout life. Prior to the COVID-19 pandemic, PIV3 accounted for approximately 60% of detected PIVs; however, post-pandemic PIV3 detection increased to approximately 99%, subsequently increasing its pervasiveness and the risk for advanced respiratory disease hospitalizations. With no approved therapy or vaccine, PIV3 continues to spread and cause severe respiratory complications in susceptible populations.

PIV3 is a negative sense, single-stranded RNA virus in the family Paramyxoviridae. The genome encodes six viral proteins, with two surface glycoproteins initiating viral infection: the fusion (F) protein and the hemagglutinin-neuraminidase (HN) protein. The HN protein attaches to the host cell by interacting with its cognate receptor, sialic acid, which in turn initiates F to refold, beginning the fusion process. The PIV3 F protein has similar functions to other class I fusion proteins, in that it initiates fusion between the virion and the host cell membrane, subsequently allowing the virus to enter and infect the host cell.

PIV3 HN is a type II membrane protein that exists as a covalently linked dimer composed of a stalk and head region. In addition to activating the F protein for viral fusion, the head domain of PIV3 HN contains an active site necessary for both sialic acid binding and cleavage, making it necessary for viral entry as well as viral release, and therefore an excellent target for mAb development. It is hypothesized that, following receptor binding to sialic acid, HN may activate F through a loop that extends into a hydrophobic pocket within the apex of F, lowering the activation barrier and triggering F to undergo conformational changes that fuse the viral and cell membranes together. After viral replication is complete within the host cell cytoplasm and viral progeny is preparing to bud from the cell, HN cleaves sialic acid from carbohydrate chains, releasing viral progeny from the host cell. These functions of HN create multiple targets for clinical intervention, with the potential to prevent receptor binding, viral fusion, or egress. While the F protein is essential for viral infection, the diverse activities of HN present opportunities for more robust treatments.

Isolation of PIV3 F-specific mAbs that neutralize PIV3 has identified several antigenic sites. For example, mAb PIA174 binds across all three protomers at the apex of PIV3 F, blocking HN interaction and subsequent fusion. Recently, cross-binding and cross-neutralizing mAbs targeting PIV1 and PIV3 F proteins have been identified, namely mAb 3′1, which protects from PIV3 challenge in vivo. PIV1 commonly causes croup in infants and young children, and serological evidence has shown it to be the second-most frequently circulating PIV. PIV1 and PIV3 HN proteins share 49% sequence homology, which suggests the potential for cross-reactive mAb binding.

With PIV3 and PIV1 circulation persisting, the lack of treatments and vaccines against PIVs leaves the susceptible populations entirely vulnerable to infection and enhanced disease. Despite advances in PIV treatment and prevention research, there are still no human monoclonal antibodies that bind to the parainfluenza virus 3 hemagglutinin-neuraminidase protein. Furthermore, there are no treatments or vaccines for PIV3 and other PIVs. These needs and other needs are satisfied by the present disclosure.

### SUMMARY

In accordance with the purpose(s) of the present disclosure, as embodied and broadly described herein, the disclosure, in one aspect, relates to isolated human antibodies that bind to at least one protein from a parainfluenza virus (PIV), including, but not limited to, PIV3 and PIV1, pharmaceutical compositions comprising same, methods of treating or preventing PIV infection using same, and methods for detecting PIV in a sample from a subject using same.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1F show assessment of potent neutralizing mAbs against PIV3 and epitope discovery on the head domain of the PIV3 HN protein. (FIG. 1A) ELISA binding curves data displays mAb binding with high affinity to the head domain of HN. Data points are the average of four technical replicates from one experiment and are representative of two biological replicates. Error bars represent the standard deviation. (FIG. 1B) Plaque reduction neutralization test curves for the nine mAbs. Data points are the average of three technical replicates from one experiment and are representative of two biological replicates. Error bars represent the standard deviation. (FIG. 1C) Summary of the half maximal effective concentration (EC₅₀) values and half maximal inhibitory concentration (IC₅₀) values calculated from (FIG. 1A) and (FIG. 1B). (FIG. 1D) PIV3-infected LLC-MK2 cells were stained with anti-PIV3 HN mAbs PIV3HN-05 and PIV3HN-09 conjugated to PE. Anti-F mAb PIA174 was used as a positive control, and a mAb against human metapneumovirus, MPV467, was used as a negative control. Both anti-HN mAbs and anti-F mAbs were detected on virally infected cells. Data represent the results from one technical replicate and are representative of two biological replicates. (FIG. 1E) Epitope binning was performed via biolayer interferometry (BLI) by loading His-tagged HN protein onto an anti-HIS sensor, and then associating the sensor with one mAb followed by a second mAb. (FIG. 1F) Three distinct epitopes on the PIV3 HN protein were discovered from the competitive binding to HN by the mAbs. Data indicate binding of the second antibody to the sensor in the presence of the first antibody. Values in black boxes describe mAbs with high levels of competition (530), whereas values in white boxes describe mAbs that do not compete for binding (>70).

FIG. 2 shows fusion inhibition of mAbs. Fusion inhibition of mAbs from each site was assessed by transfecting Vero-SLAM cells with either HN or F and GFP 3 hrs prior to adding mAb. Syncytia formation was visualized 24 hours following transfection and fusion inhibition was evaluated by comparison to the PBS-treated control. Previously identified anti-F mAb PIA174 was used as a positive control. PBS-treated cells were used as a negative control to show standard levels of syncytia. Results shown are images from one technical replicate from one experiment and are representative of 3 biological replicates.

FIGS. 3A-3E show complement recruitment and PIV1 cross-neutralization capabilities of mAbs. (FIG. 3A) Complement deposition was assessed due to its importance in aiding mAbs in preventing viral replication. Complement has several functions, including formation of the membrane attack complex (MAC), and initiation of cell phagocytosis or cellular cytotoxicity. (FIG. 3B) HN-coated beads were stained with individual mAbs before introducing the C3 complement protein. Deposition of the C3b protein to HN-coated beads stained with mAbs was measured by comparison to C3b deposition alone. Data points are the results of three technical replicates from one experiment and the data are representative of two biological replicates. Error bars represent the standard deviation. (**P<0.0086) (FIG. 3C) PRNT was performed with added guinea pig complement at a 1:1000 dilution. All except two mAbs had increased neutralization potency with added complement, indicated by a decreased IC₅₀ value. Each data point is the IC₅₀ value from one neutralization experiment with or without complement, and the data are representative of two biological replicates. (FIG. 3D) One mAb from each epitope was evaluated for hemagglutination inhibition (HI) using guinea pig red blood cells. Data are the results from one experiment representative of two biological replicates. (FIG. 3E) One mAb, PIV3HN-05, cross-neutralized parainfluenza virus type 1 (PIV1) in vitro with an IC₅₀ value of 180 ng/mL. The PIV1/3 cross-neutralizing mAb, 3×1, was used as a positive control. Data points are the average of three technical replicates from one experiment and are representative of two biological replicates. Error bars represent the standard deviation.

FIGS. 4A-4C show structural insights into the non-protective Site 1 epitope of the PIV3 HN protein. (FIG. 4A) X-ray crystal structure displaying PIVHN-13 Fab binding to the dimer interface of the head domain of the PIV3 HN protein, determined to 2.4 Å. PIV3 HN monomers, the PIVHN-13 heavy chain, and the light chain are shown. The previously proposed interaction site between the PIV3 F and PIV3 HN proteins is highlighted in gold and the sialic acid active site is highlighted in magenta. (FIG. 4B) Structures of the PIVHN-13 Fab, the PIV3 HN dimer, and the pre-fusion PIV3 F (PDB: 6MJZ) protein were fit into the PIV3 F-HN interaction map (EMDB: 27550). Site 1 on PIV3 HN contains no interactions with the PIV3 F-PIV3 HN interaction site. (FIG. 4C) Interactions between the heavy and light chain of PIVHN-13 Fab and both PIV3 HN monomers were identified. Top: CRDH3 forms a hydrophobic pocket surrounded by hydrogen bonds between CRD2 residues Gly58 and Ser64 with PIV3 HN residues Ser122 and Leu124, respectively. Hydrophobic regions are highlighted with light gray surfaces, with decreasing hydrophobicity depicted in darker gray. Middle: CDRH2 and FRH3 in the Fab heavy chain form various hydrogen bonds with the PIV3 HN monomer. All hydrogen bonds are formed with one monomer except for one between His63 on the heavy chain and Asn139 on the second HN monomer. Bottom: Hydrogen bonds between the light chain of the Fab and one HN monomer are found in the CDRL3 and FRL1. Hydrogen bonds are shown in dotted lines.

FIGS. 5A-5D show structural insights into the protective Site 2-specific mAb PIV3HN-05. (FIG. 5A) Cryo-EM map of PIV3HN-05 Fab complexed with HN, with PIV3HN-05 Fab binding to the apical surface of HN. (FIG. 5B) When fit into a map of F (PDB: 6MJZ) and HN interaction (EMDB: 27550), the PIV3HN-05 CDRH3 does not spatially interact with the active site of HN but fits directly within the HN active site. (FIG. 5C) Overlay of the Alphafold3 predicted structure (grey) and experimentally determined structure (HC: indigo, LC: mauve) of PIV3HN-05 Fab complexed to HN. (FIG. 5D) Interactions of the PIV3HN-05 CDRH3 with the PIV3 HN protein in the sialic acid binding pocket. Two hydrogen bonds are indicated with black dotted lines. Hydrophobic regions are highlighted with lighter gray translucent surfaces, with decreasing hydrophobicity depicted in darker gray. HN residues associated with sialic acid receptor interactions are labeled. Difluorosialic acid atoms fit into the HN model (PDB: 4WEF) are depicted in black.

FIGS. 6A-6D show mAb treatment of Syrian hamsters before and after PIV3 challenge. (FIG. 6A) Syrian hamsters (n=10) were treated with 10 mg/kg of one mAb binding to each identified site on HN 24 hours prior to intranasal PIV3 challenge. Lungs and nasal wash were collected four days following viral challenge. (FIG. 6B) Plaque assays revealed decreased viral load in the lungs with prophylactically PIV05- and PIV09-treated hamsters. (FIG. 6C) Syrian hamsters (n=5) were treated 24 hours after intranasal PIV3 challenge. Lungs and nasal wash were collected four days following viral challenge. (FIG. 6D) Viral load in the lungs of PIV05 was significantly lower than the mock-treated group. (*P<0.04, **P<0.0098, ***P<0.0002).

FIGS. 7A-7E show flow sorting of antigen-specific B cells using single antigen staining. PBMCs were Ficoll-isolated from healthy human adult donors and flow sorted to obtain live CD19⁺, IgD/A/M⁻, PIV3 HN⁺ B cells. (FIG. 7A) Lymphocytes were selected from the original PBMC sample. (FIG. 7B) Lymphocytes negative for the live/dead DAPI stain were selected. (FIG. 7C) Live lymphocytes positive for CD19 conjugated to APC were selected. (FIG. 7D) CD19⁺ live lymphocytes negative for IgD/A/M conjugated to FITC were selected. (FIG. 7E) The final population of live CD19⁺, IgD/A/M⁻ lymphocytes positive for PIV3 HN conjugated to BV605 were sorted onto a 3T3 feeder layer in Media A supplemented with CpG and cyclosporin A and incubated for 2 weeks prior to antigen screening.

FIGS. 8A-8E show flow sorting of antigen-specific B cells using dual antigen staining. PBMCs were sorted under the same conditions as in FIGS. 7A-7E, with an additional PIV3 HN conjugated to a second fluorophore to sort double-antigen positive cells.

FIG. 9 shows hemagglutination inhibition (HI) assay performed with guinea pig red blood cells (RBCs). Each mAb was serially diluted and incubated with 4 HA units of PIV3 before adding to RBCs. Inhibition of RBC hemagglutination was measured and the mean inhibitory concentration (MIC) of each mAb was calculated based on the last dilution of mAb to inhibit hemagglutination.

FIGS. 10A-10E show size exclusion chromatography and negative stain electron microscopy of PIV3 HN+Fab complexes. (FIG. 10A) Size exclusion chromatogram of PIV3 HN, which eluted at approximately 14-15 mL on the Superdex S200, 10/300 column. (FIG. 10B) Size exclusion chromatograph of PIV3HN-05 Fab, which eluted at approximately 15 mL on the Superdex S200, 10/300 column. (FIG. 10C) Size exclusion chromatogram of PIV3 HN complexed with PIV3HN-13 Fab. The protein+Fab complex eluted at approximately 12 mL, and excess Fab eluted at approximately 15 mL on the Superdex S200, 16/600 column. (FIG. 10D) Size exclusion chromatogram of PIV3 HN complexed with PIV3HN-05 Fab and PIV3HN-13 Fab. The protein+Fabs complex eluted at approximately 11 mL, and excess Fab eluted at approximately 15 mL. (FIG. 10E) Negative stain electron micrographs of PIV3 HN (left), PIV3 HN complexed with PIV3HN-05 Fab (middle), and PIV3 HN complexed with PIV3HN-05 Fab and PIV3HN-13 Fab. Scale bar=50 nm.

FIG. 11 shows workflow of the cryo-EM data processing. Cryosparc readouts from the nonuniform refinement are provided along with a calculated local resolution map.

FIG. 12 shows a snapshot of the electron density for the PIV3HN-05-PIV3HN-13-PIV3 HN cryoEM structure. The CDRH3 of PIV3HN-05 binding into the sialic acid binding site is shown on the left. The map is contoured to 3.1 rmsd in COOT.

FIGS. 13A-13B show ELISA binding and virus neutralization of isolated mAbs. (FIG. 13A) ELISA binding curves of isolated mAbs, 5217-2 and 5217-9, against the PIV3 HN protein. Optical density at 405 nm (OD405) was measured using a microplate reader. Data points are the average of four technical replicates from one experiment and are representative of two biological replicates. The data is presented using mean values ±standard deviation. (FIG. 13B) Plaque reduction neutralization assay of the two isolated mAbs against PIV3. Data points are the average of three technical replicates from one experiment and are representative of two biological replicates. The data is presented using mean values ±standard deviation.

FIG. 14 shows biolayer interferometry (BLI) epitope binning analysis of novel mAbs against previously characterized mAbs. A heat map showing the results from a binning experiment with the six of the previously characterized mAbs (PIV3HN-13, PIV3HN-05. PIV3HN-11, PIV3HN-03, PIV3HN-04, PIV3HN-09) and the two new mAbs (5217-2 and 5217-9). Black indicates complete competition (533%), gray indicates partial competition (34-66%), and white indicates no competition (>67%). The formula to calculate the competition percentage is as follows % Competition=(signal from first mAb with second mAb/signal from first mAb)×100%. The addition of mAb 5217-2 led to the discovery of a new epitope on PIV3 HN, designated as Site 4, while mAb 5217-9 bound to the previously characterized Site 1. Both epitopes are highlighted.

FIGS. 15A-15D show cryo-EM structure of Site 3 mAb PIV3HN-09 binding to the PIV3 HN head. (FIG. 15A) Cryo-EM density map of a PIV3 HN dimer bound to PIV3HN-09 Fabs and PIV3HN-13 Fabs. (FIG. 15B) Alternative view of the atomic model (ribbon representation) fitted into the cryo-EM density (transparent surface), demonstrating a good model to map fit. (FIG. 15C) Surface representation of the HN proteins with bound Fab fragments, PIV3HN-09 heavy chains are colored dark gray and light chains are light gray. (FIG. 15D) Detailed view of the interaction interface between the HN protein and the PIV3HN-09 heavy chain, highlighting key interactions. Hydrogen bonds are depicted as dotted lines, with distances labeled in angstroms (Å). The top panel shows a side view of the binding interface, while the bottom panel provides a top-down perspective.

FIGS. 16A-16D show cryo-EM structure of Site 4 mAb 5217-2 binding to the PIV3 HN head. (FIG. 16A) Cryo-EM density map of a PIV3 HN dimer bound to 5217-2 Fabs (red) and PIV3HN-09 Fabs. (FIG. 16B) Alternative view of the atomic model (ribbon representation) fitted into the cryo-EM density (transparent surface), demonstrating a good model to map fit. (FIG. 16C) Surface representation of the HN proteins with bound Fab fragments. (FIG. 16D) Detailed view of the interaction interface between the HN protein and the 5217-2 heavy and light chains, highlighting key contact residues. Hydrogen bonds are depicted as dotted lines, with distances labeled in angstroms (Å). The top panel shows a side view of the binding interface, while the bottom panel provides a top-down perspective.

FIGS. 17A-17D show an updated cryo-EM structure of Site 2 mAb PIV3HN-05 binding to the PIV3 HN head and evaluation of cross-reactivity in a PIV1 viral challenge model. (FIG. 17A) Cryo-EM density map of a PIV3 HN dimer bound to PIV3HN-05 Fabs and PIV3HN-13 Fabs. (FIG. 17B) Detailed view of the interaction interface between the HN protein and the HCDR3 loop of PIV3HN-05 Fab. The Fab does not directly interact with the HN sialic acid active site (residues 234-323) but inserts into the active site, occupying the space. Hydrogen bonds are depicted as dotted lines, with distances labeled in angstroms (Å). (FIG. 17C) The left panel shows the overall structure of the PIV3 HN head domain in complex with the PIV3HN-05 Fab. To evaluate the potential mechanism behind PIV1 cross-reactivity, the PIV1 HN ectodomain was structurally aligned with the PIV3 HN head, and the binding site of the PIV3HN-05 Fab was compared between the two. (FIG. 17D) In vivo evaluation of PIV3HN-05 against PIV1 challenge. Top: Experimental timeline of the hamster study. Syrian hamsters (n=10) received a single dose of PIV3HN-05 (10 mg/kg) one day prior to the PIV1 viral challenge. Lungs were harvested four days post-challenge, and viral titers were assessed via plaque assay. Bottom: Results of the plaque assay show a reduction in lung viral load in the PIV3HN-05 treated group compared to the PBS control.

FIGS. 18A-18B show structural mapping of Site 1-4 Fabs bound to the PIV3 HN head domain in complex with the PIV3 F protein. (FIG. 18A) A model showing the PIV3 HN head domain simultaneously bound by the four site-specific Fabs: Site 1 PIV3HN-13, Site 2 PIV3HN-05, Site 3 PIV3HN-09, and Site 4 5217-2. This model provides a comprehensive representation of the epitope location on the HN head, illustrating the relative position and accessibility of each site. (FIG. 18B) A model of the PIV3 HN head domain with Site 1-4 Fabs bound in the context of the PIV3 F protein, generated using a structure of prefusion F (PDB: 6MJZ) and the HN-F interaction map (EMB: 27550). This model shows the orientation and relative positioning of each Fab when the HN is engaged with the F protein.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Human parainfluenza viruses (HPIVs) are a leading cause of acute respiratory infections (ARIs) in children, immunocompromised individuals, and the elderly. While HPIVs can affect individuals of all ages, the highest incidence occurs in children under the age of 5, contributing significantly to infant morbidity and mortality. HPIVs can cause both upper and lower respiratory tract infections, typically presenting with cold-like symptoms. In more severe cases, sometimes resulting in hospitalization, HPIVs can cause croup, bronchitis, and pneumonia. There are five known serotypes of parainfluenza viruses that infect humans: HPIV1, HPIV2, HPIV3, HPIV4a, and HPIV4b. Among the different serotypes, PIV3 has been associated with the highest hospitalization rates, reflecting its potential for causing severe respiratory illness. PIV1 follows closely behind, also contributing to hospital admissions in children under 5 years of age. Notably, children under one year of age exhibited the highest rate of PIV infection, reaching a striking 45.5%. Among these cases, PIV3 was the most prevalent, accounting for 58.7% followed by PIV1 at 36.4%.

Currently, there are no approved vaccines for HPIVs, largely due to challenges in eliciting long-lasting and protective immune responses, particularly in seronegative children. However, increasing attention is being directed toward monoclonal antibodies as a potential therapeutic option, given their demonstrated safety in humans, high specificity to the virus, and ability to effectively engage the immune system. Significant advances has been made in monoclonal antibody research for respiratory viruses such as respiratory syncytial virus (RSV) and influenza. For RSV, palivizumab, a monoclonal antibody targeting the fusion (F) protein, has been shown to significantly reduce the rate of hospitalization in high-risk premature infants and children with congenital lung disease. The success of monoclonal antibody-based therapies offers a promising strategy for preventing and treating HPIV infections, especially in vulnerable populations where vaccine responses can be limited.

The HPIVs are negative sense, single-stranded RNA viruses belonging to the Paramyxovirdae family. PIV1 and PIV3 are classified under the genus Respirovirus, while PIV2 and PIV4 fall within the genus Rubulavirus. The RNA genome of PIV3 encodes six essential proteins: nucleocapsid protein (NP), phosphoprotein (P), matrix protein (M), fusion glycoprotein (F), hemagglutinin neuraminidase glycoprotein (HN), and RNA polymerase (L). The HN and F surface glycoproteins play critical roles in initiating infection. The HN protein binds to sialic acid residues on the surface of host epithelial cells, facilitating viral attachment, while the F protein mediates the fusion of the viral envelope with the host cell membrane, enabling viral entry. Both the HN and F proteins are major targets for neutralizing antibodies, making them key candidates for therapeutic and vaccine development.

The HN protein is a homotetrameric, type II integral membrane protein composed of a stalk region and a globular head domain. 9 Unlike many other receptor-binding glycoproteins, HN possesses dual functionality: it uses hemagglutinin to bind sialic acid on host cells and neuraminidase to cleave sialic acid and release viral attachment. In addition to mediating attachment to sialic acid-containing receptors, the HN protein plays a critical role in activating the F (fusion) protein. Upon activation, the F protein undergoes a conformational change that exposes its buried fusion peptide, enabling the fusion of the viral and host cell membranes. The virus relies heavily on the HN protein functions including receptor binding, receptor cleavage, and F protein activation making it a strategic target for interventions aimed at disrupting receptor binding thus preventing membrane fusion. While the F protein has been the focus of many antiviral strategies, the multifunctional nature of the HN protein highlights its potential as a more advantageous therapeutic target.

The absence of effective treatments allows human parainfluenza viruses to remain a significant threat to vulnerable populations. monoclonal antibodies targeting the hemagglutinin-neuraminidase (HN) protein of PIV3. Herein are disclosed mAbs that demonstrate both high binding affinity and potent neutralizing activity for the HN protein of PIV3. Each of these mAbs were found to target a distinct epitope on the HN protein, and the structural features of these epitopes were characterized.

Disclosed herein are human mAbs against the PIV3 HN protein. In one aspect, using these mAbs, three distinct epitopes on the HN protein have successfully been mapped, the functional characteristics of mAbs binding to each epitope including fusion inhibition and complement deposition have been assessed, the structural features of two epitopes found on the PIV3 HN protein have been determined, and potent neutralizing mAbs as prophylactic and therapeutic treatments in vivo have been evaluated. In a further aspect, one mAb decreased PIV3 infection in vivo and binds to the sialic acid active site on HN, presumably preventing binding and fusion of the virus. In a further aspect, this mAb introduces promising insights into HN-targeted treatments and vaccines for the clinical realm.

### Isolated Human Antibodies

In one aspect, disclosed herein is an isolated human antibody that binds to at least one protein from a parainfluenza virus (PIV). In some aspects, the antibody can be a monoclonal antibody. In a further aspect, mixtures of disclosed monoclonal antibodies are also contemplated and should be considered disclosed. In an aspect, the PIV can be PIV1, PIV2, PIV3, or PIV4, or any combination thereof. In another aspect, the PIV is PIV3. In another aspect, the PIV is PIV1. In some aspects, the antibody can cross-neutralize more than one PIV. In one aspect, the protein can be an envelope protein such as, for example, hemagglutinin-neuraminidase or fusion protein.

In a further aspect, the heavy chain of the isolated human antibody has an amino acid sequence selected from SEQ ID NOs. 62-65, SEQ ID NOs. 66-70, or SEQ ID NO. 134, 136, 138, 140, 142, 144, 146, 148, 150, or 152. In another aspect, the light chain of the isolated human antibody has an amino acid sequence selected from SEQ ID NOs. 80-83, SEQ ID NOs. 85-88, or SEQ ID NO. 135, 137, 139, 141, 143, 145, 147, 149, 151, or 153. In one aspect, the heavy chain has SEQ ID NO. 64 and the light chain has SEQ ID NO. 52, corresponding to PIV3HN-05. In a further aspect, this combination of heavy chain and light chain is a potent neutralizing antibody and can also protect against PIV1 disease as well as PIV3. Also disclosed herein are nucleic acids encoding the isolated human antibodies. In an aspect, the nucleic acids can include DNA, RNA, or a combination thereof. In one aspect, the nucleic acid includes first nucleic acid sequence encoding a heavy chain and a second nucleic acid sequence comprising a light chain. In a further aspect, the first nucleic acid sequence can be selected from SEQ ID NO. 114, 116, 118, 120, 122, 124, 126, 128, 130, or 132 and the second nucleic acid sequence is selected from SEQ ID NO. 115, 117, 119, 121, 123, 125, 127, 129, 131, or 133.

Furthermore, disclosed herein are viral vectors including the nucleic acids. In aspects where the disclosed nucleic acid is administered therapeutically, the subject's own cellular machinery can be used to manufacture the antibody sequences.

In some aspects, the isolated human antibody includes one or more introduced mutations in order to increase effectiveness of treatment or prevention of PIV in a subject. In one aspect, the isolated human antibody has at least one mutation in a heavy chain of the isolated human antibody in an amino acid sequence selected from SEQ ID NOs. 62-65, SEQ ID NOs. 66-70, or SEQ ID NO. 134, 136, 138, 140, 142, 144, 146, 148, 150, or 152, or at least one mutation in a light chain of the isolated human antibody in an amino acid sequence selected from SEQ ID NOs. 80-83, SEQ ID NOs. 85-88, or SEQ ID NO. 135, 137, 139, 141, 143, 145, 147, 149, 151, or 153, or both. In an aspect, the at least one mutation increases complement deposition activity, antibody-dependent phagocytosis, antibody-dependent cellular cytotoxicity, antibody half-life, or any combination thereof.

In some aspects, the isolated human antibody can be bispecific or trispecific.

In another aspect, the isolated human antibody has an IC₅₀ against the PIV of lower than 200 ng/mL, lower than 100 ng/mL, or lower than 50 ng/mL. In another aspect, the isolated human antibody has an EC₅₀ against the PIV of about 50 ng/mL

### Pharmaceutical Compositions and Methods for Passive Immunization

In another aspect, disclosed herein are pharmaceutical compositions including the disclosed isolated human antibodies, nucleic acids, and/or viral vectors and a pharmaceutically acceptable carrier.

In still another aspect, disclosed herein is a method for passive immunization of a subject against a PIV, the method including administering at least one disclosed isolated human antibody or the disclosed pharmaceutical composition to the subject. In another aspect, the passive immunization can be prophylactic, therapeutic, or both. In one aspect, the pharmaceutical composition can be formulated to be administered intranasally, by inhalation, or by injection, including, but not limited to intramuscular, intravenous, intraperitoneal, or subcutaneous injection.

In an aspect, the subject is human. In another aspect, performing the method inhibits viral fusion with a plurality of cells in the subject. In still another aspect, performing the method induces complement deposition. In one aspect, performing the method inhibits hemagglutination of red blood cells in the subject. In another aspect, performing the method prevents replication of the PIV in the subject.

### Methods for Detecting Parainfluenza Viruses

In still another aspect, disclosed herein is a method for detecting a PIV in a subject, the method including at least the steps of:
(a) obtaining a sample from the subject;
(b) contacting the sample with a disclosed isolated human antibody; and
(c) detecting a signal from the sample;
   wherein presence of the signal indicates that the PIV is present in the subject and wherein absence of the signal indicates that the PIV is not present in the subject.

In another aspect, the method is or includes an ELISA assay. In still another aspect, the subject is a human. In one aspect, the sample can be a nasal swab or lung fluid. In still another aspect, the signal can be selected from a color change, a fluorescence signal, a luminescence signal, or any combination thereof. Other useful signals and methods of measuring the same useful in the disclosed process include surface plasmon resonance (SPR), where detection of the signal is observed as a resonance unit change, and biolayer interferometry (BLI), wherein detection of the signal is observed as a wavelength shift change.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a parainfluenza virus,” “an epitope,” or “an antibody,” includes, but is not limited to, mixtures or combinations of two or more such parainfluenza viruses, epitopes, or antibodies, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of an antibody refers to an amount that is sufficient to achieve the desired improvement in the property modulated by the formulation component, e.g. achieving the desired half maximal inhibitory concentration (IC₅₀). The specific level in terms of wt % in a composition required as an effective amount will depend upon a variety of factors including the particular parainfluenza virus infecting a subject, amount and type of any adjuvants or other pharmaceuticals present, viral load, subject body weight, and concurrent infection with any other viral diseases.

As used herein, the terms “optional” or “optionally” means that the subsequently described event or circumstance can or cannot occur, and that the description includes instances where said event or circumstance occurs and instances where it does not.

As used herein, “bispecific” refers to an antibody that is capable of binding to two different epitopes or antigens at the same time, while a “trispecific” antibody can bind to at least three different epitopes or antigens at the same time. In an aspect, the disclosed isolated human antibodies can be bispecific or trispecific.

Unless otherwise specified, temperatures referred to herein are based on atmospheric pressure (i.e. one atmosphere).

### Pharmaceutical Compositions

As used herein, “administering” can refer to an administration that is intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as preventing or partially preventing PIV3 or a symptom of PIV3. The effect can be therapeutic in terms of a partial or complete cure of a disease, condition, symptom or adverse effect attributed to the disease, disorder, or condition. The term “treatment” as used herein can include any treatment of PIV3 in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent.

Described herein are human monoclonal antibodies and pharmaceutical compositions containing the same that have therapeutic or clinical utility. Also described herein are methods of synthesizing the human monoclonal antibodies. Also described herein are methods of administering the human monoclonal antibodies to a subject in need thereof. In some aspects, the subject can have a parainfluenza virus such as, for example, PIV3 Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

In a further aspect, the disclosed pharmaceutical compositions comprise a therapeutically effective amount of at least one disclosed human monoclonal antibody. The disclosed pharmaceutical compositions include those suitable for oral, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, and/or parenterally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed human monoclonal antibody may be formulated into various pharmaceutical forms for administration purposes.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a human monoclonal antibody of the present disclosure as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for nasal and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The human monoclonal antibodies described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable human monoclonal antibody will be in a form suitable for oral, nasal, intravenous injection, intramuscular injection, or parenteral administration. Carriers include solids or liquids, and the type of carrier is chosen based on the type of administration being used. The compounds may be administered as a dosage that has a known quantity of the compound.

Pharmaceutical compositions of the present disclosure can be suitable for injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions, or suspensions, including saline and buffered media. Parenteral vehicles can include mannitol, normal serum albumin, sodium chloride solution, Ringer's dextrose, dextrose and sodium chloride, lactated Ringer's, and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment conditions that require modulation of PIV3 viral activity, an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The present disclosure is further directed to a method for the manufacture of a medicament for modulating PIV3 activity (e.g., treatment of PIV3 infection) in mammals (e.g., humans) comprising combining one or more disclosed human monoclonal antibodies, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed human monoclonal antibody and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

As already mentioned, the present disclosure also relates to a pharmaceutical composition comprising a disclosed human monoclonal antibody, The present disclosure also relates to such a composition for use as a medicine. The present disclosure also relates to an additional antiviral therapeutic agent, as a combined preparation for simultaneous, separate, or sequential use in the treatment or prevention of a condition in a mammal, including a human, the treatment or prevention of which is affected or facilitated by the modulatory effect of the disclosed compound and the additional therapeutic agent. The different drugs of such a combination or product may be combined in a single preparation together with pharmaceutically acceptable carriers or diluents, or they may each be present in a separate preparation together with pharmaceutically acceptable carriers or diluents.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered aspects, which should not be confused with the claims.

Aspect 1. An isolated human antibody that binds to at least one protein from a parainfluenza virus (PIV).

Aspect 2. The isolated human antibody of aspect 1, wherein the antibody is a monoclonal antibody.

Aspect 3. The isolated human antibody of aspect 1 or 2, where the parainfluenza virus comprises PIV1, PIV2, PIV3, PIV4, or any combination thereof.

Aspect 4. The isolated human antibody of aspect 3, wherein the parainfluenza virus is PIV3.

Aspect 5. The isolated human antibody of aspect 3, wherein the parainfluenza virus is PIV1.

Aspect 6. The isolated human antibody of any one of aspects 1-5, wherein the at least one protein comprises an envelope protein.

Aspect 7. The isolated human antibody of aspect 6, wherein the envelope protein comprises hemagglutinin-neuraminidase or fusion protein.

Aspect 8. The isolated human antibody of any one of aspects 1-7, wherein a heavy chain of the isolated human antibody has an amino acid sequence selected from SEQ ID NOs. 62-65, SEQ ID NOs. 66-70, or SEQ ID NO. 134, 136, 138, 140, 142, 144, 146, 148, 150, or 152.

Aspect 9. The isolated human antibody of any one of aspects 1-8, wherein a light chain of the isolated human antibody has an amino acid sequence selected from SEQ ID NOs. 80-83, SEQ ID NOs. 85-88, or SEQ ID NO. 135, 137, 139, 141, 143, 145, 147, 149, 151, or 153.

Aspect 10. The isolated human antibody of any one of aspects 1-9, wherein the isolated human antibody has a light chain amino acid sequence of SEQ ID NO. 82 and a heavy chain amino acid sequence of SEQ ID NO. 64.

Aspect 11. The isolated human antibody of any one of aspects 1-7, wherein the isolated human antibody has at least one mutation in a heavy chain of the isolated human antibody in an amino acid sequence selected from SEQ ID NOs. 62-65, SEQ ID NOs. 66-70, or SEQ ID NO. 134, 136, 138, 140, 142, 144, 146, 148, 150, or 152, or at least one mutation in a light chain of the isolated human antibody in an amino acid sequence selected from SEQ ID NOs. 80-83, SEQ ID NOs. 85-88, or SEQ ID NO. 135, 137, 139, 141, 143, 145, 147, 149, 151, or 153, or both.

Aspect 12. The isolated human antibody of aspect 11, wherein the at least one mutation increases complement deposition activity, antibody-dependent phagocytosis, antibody-dependent cellular cytotoxicity, antibody half-life, or any combination thereof.

Aspect 13. The isolated human antibody of any one of aspects 1-12, wherein the isolated human antibody is bispecific or trispecific.

Aspect 14. The isolated human antibody of any one of aspects 1-13, wherein the isolated human antibody has an IC₅₀ against the PIV lower than 200 ng/mL.

Aspect 15. The isolated human antibody of any one of aspects 1-13, wherein the isolated human antibody has an IC₅₀ against the PIV lower than 100 ng/mL.

Aspect 16. The isolated human antibody of any one of aspects 1-13, wherein the isolated human antibody has an IC₅₀ against the PIV lower than 50 ng/mL.

Aspect 17. The isolated human antibody of any one of aspects 1-16, wherein the isolated human antibody has an EC₅₀ against the PIV of about 50 ng/mL.

Aspect 18. A nucleic acid encoding the isolated human antibody of any one of aspects 1-17.

Aspect 19. The nucleic acid of aspect 18, wherein the nucleic acid comprises RNA, DNA, or any combination thereof.

Aspect 20. The nucleic acid of aspect 18 or 19, wherein the nucleic acid comprises a first nucleic acid sequence encoding a heavy chain and a second nucleic acid sequence comprising a light chain.

Aspect 21. The nucleic acid of aspect 20, wherein the first nucleic acid sequence is selected from SEQ ID NO. 114, 116, 118, 120, 122, 124, 126, 128, 130, or 132.

Aspect 22. The nucleic acid of aspect 20 or 21, wherein the second nucleic acid sequence is selected from SEQ ID NO. 115, 117, 119, 121, 123, 125, 127, 129, 131, or 133.

Aspect 23. A viral vector comprising the nucleic acid of any one of aspects 20-22.

Aspect 24. A pharmaceutical composition comprising the isolated human antibody of any one of aspects 1-17, the nucleic acid of any one of aspects 18-22, or the viral vector of aspect 23, and a pharmaceutically acceptable carrier.

Aspect 25. The pharmaceutical composition of aspect 24, wherein the pharmaceutical composition is formulated to be administered intranasally, by inhalation, or by injection.

Aspect 26. The pharmaceutical composition of aspect 25, wherein the injection is intramuscular, intravenous, intraperitoneal, or subcutaneous.

Aspect 27. A method for passive immunization of a subject against a parainfluenza virus (PIV), the method comprising administering the isolated human antibody of any one of aspects 1-17, the nucleic acid of any one of aspects 18-22, the viral vector of aspect 23, or the pharmaceutical composition of any one of aspects 24-26 to the subject.

Aspect 28. The method of aspect 27, wherein the passive immunization is prophylactic, therapeutic, or both.

Aspect 29. The method of aspect 27 or 28, where the parainfluenza virus comprises PIV1, PIV2, PIV3, PIV4, or any combination thereof.

Aspect 30. The method of aspect 29, wherein the parainfluenza virus is PIV3.

Aspect 31. The method of aspect 29, wherein the parainfluenza virus is PIV1.

Aspect 32. The method of any one of aspects 27-31, wherein the subject is a human.

Aspect 33. The method of any one of aspects 27-32, wherein performing the method inhibits viral fusion with a plurality of cells in the subject.

Aspect 34. The method of any one of aspects 27-33, wherein performing the method induces complement deposition.

Aspect 35. The method of any one of aspects 27-34, wherein performing the method inhibits hemagglutination of red blood cells in the subject.

Aspect 36. The method of any one of aspects 27-35, wherein performing the method prevents replication of the PIV in the subject.

Aspect 37. A method for detecting a parainfluenza virus (PIV) in a subject, the method comprising:
(a) obtaining a sample from the subject;
(b) contacting the sample with the isolated human antibody of any one of aspects 1-17; and
(c) detecting a signal from the sample;
wherein presence of the signal indicates that the PIV is present in the subject and wherein absence of the signal indicates that the PIV is not present in the subject.

Aspect 38. The method of aspect 37, wherein the method comprises an ELISA assay.

Aspect 39. The method of aspect 37 or 38, wherein the subject is a human.

Aspect 40. The method of any one of aspects 37-39, wherein the sample comprises a nasal swab or lung fluid.

Aspect 41. The method of any one of aspects 37-40, wherein the signal comprises a color change, a fluorescence signal, a luminescence signal, a resonance unit change, a wavelength shift change, or any combination thereof.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Results

Eight Neutralizing Human mAbs were Isolated Against the Head Domain of the PIV3 HN Protein.

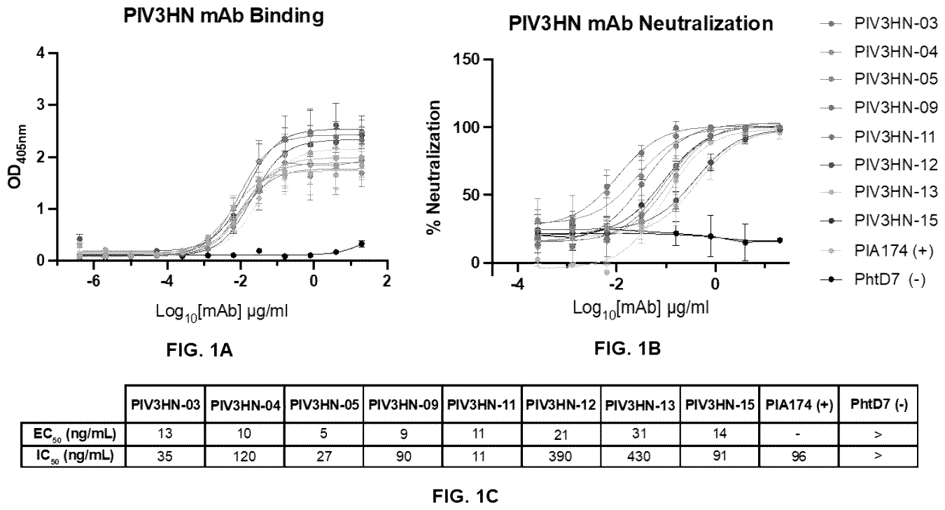

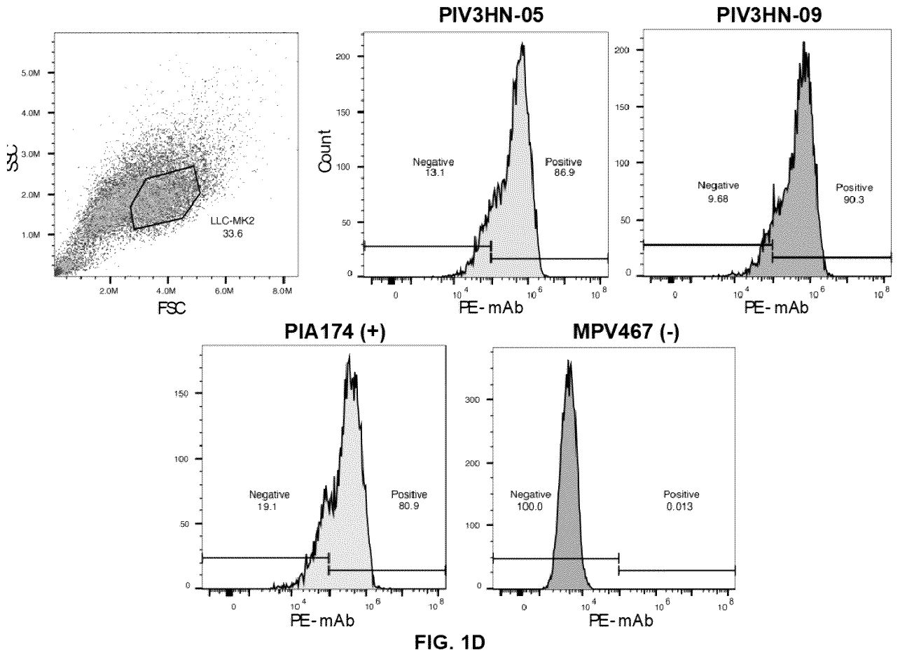

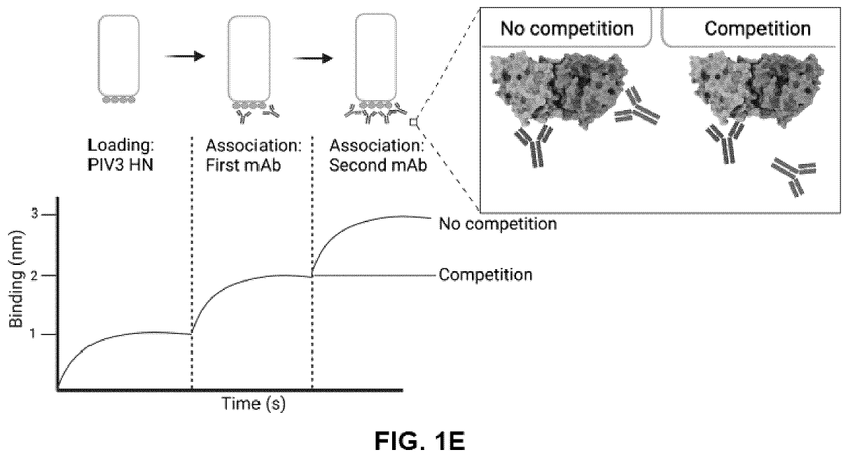

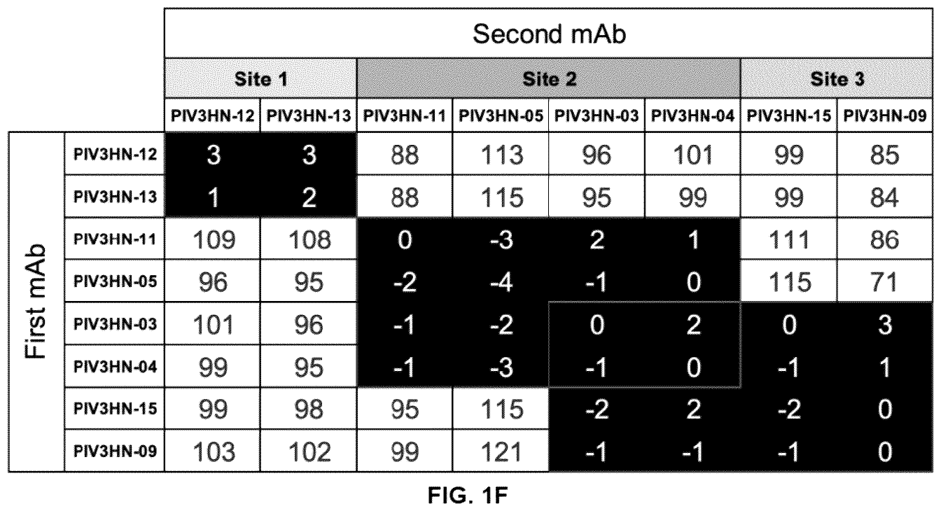

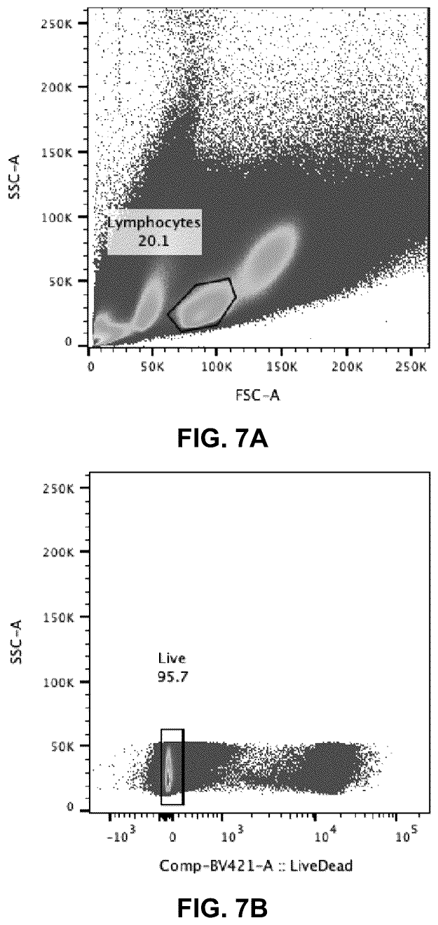

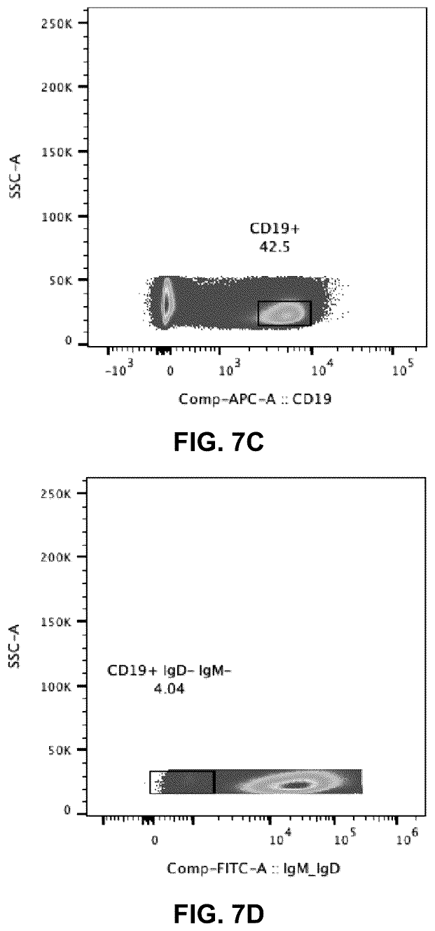

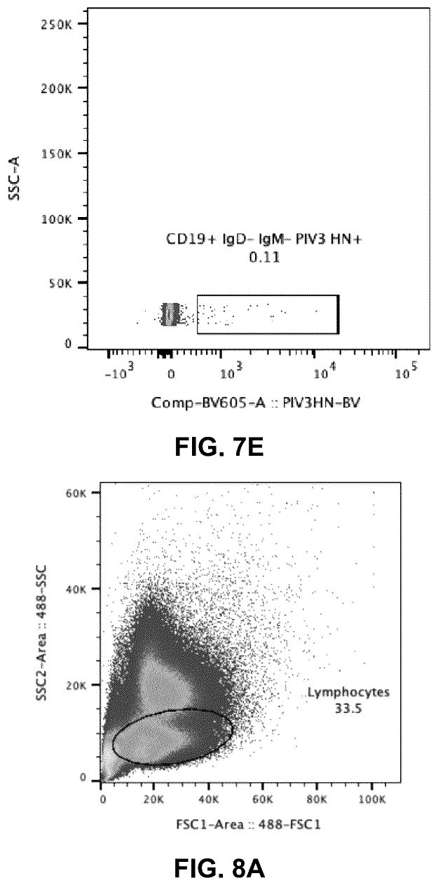

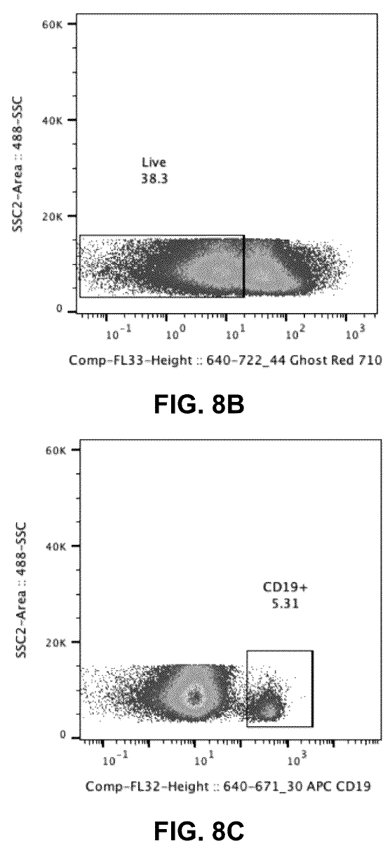

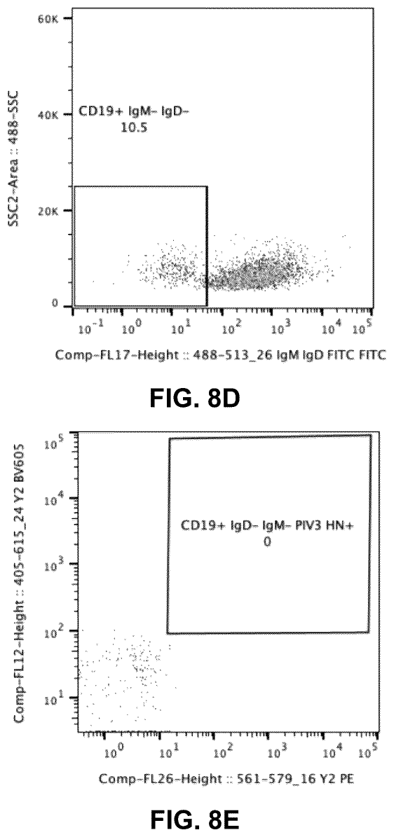

Human peripheral blood mononuclear cells (PBMCs) were isolated from blood bank leukocyte reduction filters and sorted antigen-specific B cells using fluorescent streptavidin tetramers conjugated to the head region of the PIV3 HN protein (Table 1). CD19⁺/IgA⁻/IgD⁻/IgM⁻/PIV3 HN⁺ B cells were single-cell sorted onto an irradiated NIH/3T3 cell feeder layer expressing human CD40L, a human B cell activating factor, and human interleukin-21 (hIL-21) with added CpG to stimulate B cell expansion and differentiation into antibody-secreting cells as previously described (FIGS. 7A-8E). The antibody-containing supernatant was evaluated for positive binding to recombinant PIV3 HN protein by enzyme-linked immunosorbent assay (ELISA), and RNA from positive clones was extracted, sequenced, and cloned into an expression vector (Table 2). Eight mAbs were recombinantly expressed and evaluated for binding to recombinant PIV3 HN head protein by ELISA (FIG. 1A, Table 3). The neutralizing activity of each mAb against PIV3 was also evaluated using a plaque reduction neutralization assay (PRNT) (FIG. 1B). All eight mAbs displayed HN binding activity, with effective concentrations (EC₅₀) less than 50 ng/mL (FIG. 1C). Additionally, all mAbs were able to neutralize PIV3, with mAbs PIV3HN-03, PIV3HN-05, PIV3HN-09, PIV3HN-11, and PIV3HN-15 being the most potent, displaying inhibitory concentrations (IC₅₀) lower than 100 ng/mL (FIG. 1C). Clinically available mAb therapies encompass mAbs with IC₅₀ values close to 100 ng/mL, corroborating the clinical relevancy of the identified mAbs in this study.

TABLE 1
Demographic information of adult human subjects

| Donor | Age | Sex | Race | mAb |
|---|---|---|---|---|
| Subject 1 | 59 years | Female | Caucasian | PIV3HN-03 |
| | | | | PIV3HN-04 |
| | | | | PIV3HN-05 |
| Subject 2 | 48 years | Male | Caucasian | PIV3HN-09 |
| | | | | PIV3HN-11 |
| | | | | PIV3HN-12 |
| | | | | PIV3HN-13 |
| Subject 3 | 57 years | Male | Caucasian | PIV3HN-15 |

TABLE 2
PCR primer sequences
RT-PCR (SEQ ID NOs. 1-17)

| Primer | Sequence |
|---|---|
| 5′ L-VH 1 | ACAGGTGCCCACTCCCAGGTGCAG |
| 5′ L-VH 3 | AAGGTGTCCAGTGTGARGTGCAG |
| 5′ L-VH 4/6 | CCCAGATGGGTCCTGTCCCAGGTGCAG |
| 5′ L-VH 5 | CAAGGAGTCTGTTCCGAGGTGCAG |
| 3′ HuIgG-constant | TCTTGTCCACCTTGGTGTTGCT |
| 5′ L Vk 1/2 | ATGAGGSTCCCYGCTCAGCTGCTGG |
| 5′ L Vk 3 | CTCTTCCTCCTGCTACTCTGGCTCCCAG |
| 5′ L Vk 4 | ATTTCTCTGTTGCTCTGGATCTCTG |
| 3′ Ck 543-566 | GTTTCTCGTAGTCTGCTTTGCTCA |
| 5′ L Vl 1 | GGTCCTGGGCCCAGTCTGTGCTG |
| 5′ L Vl 2 | GGTCCTGGGCCCAGTCTGCCCTG |
| 5′ L Vl 3 | GCTCTGTGACCTCCTATGAGCTG |
| 5′ L Vl 4/5/9 | GGTCTCTCTCSCAGCYTGTGCTG |
| 5′ L Vl 6 | GTTCTTGGGCCAATTTTATGCTG |
| 5′ L Vl 7 | GGTCCAATTCYCAGGCTGTGGTG |
| 5′ L Vl 8 | GAGTGGATTCTCAGACTGTGGTG |
| 3′ CI | CACCAGTGTGGCCTTGTTGGCTTG |

2^{ND} PCR (SEQ ID NOs. 18-31)

| Primer | Sequence |
|---|---|
| 5′ VH3a-sense | SARGTGCAGCTCGTGGAG |
| 5′ VH3b-sense | GAGGTGCAGCTGTTGGAG |
| 5′ VH1/5/7-sense | CTGCAACCGGTGTACATTCCGAGGTGCAGCTGGTGCAG |
| 5′ VH4-sense | CTGCAACCGGTGTACATTCCCAGGTGCAGCTGCAGGAG |
| 3′ Cgamma (IgG) | AGTAGTCCTTGACCAGGCAGCCCAG |
| 5′ Pan Vk | ATGACCCAGWCTCCABYCWCCCTG |
| 3′ Ck 494-516 | GTGCTGTCCTTGCTGTCCTGCT |
| 5′ AgeI Vl 1 | CTGCTACCGGTTCCTGGGCCCAGTCTGTGCTGACKCAG |
| 5′ AgeI Vl 2 | CTGCTACCGGTTCCTGGGCCCAGTCTGCCCTGACTCAG |
| 5′ AgeI Vl 3 | CTGCTACCGGTTCTGTGACCTCCTATGAGCTGACWCAG |
| 5′ AgeI Vl 4/5/9 | CTGCTACCGGTTCTCTCTCSCAGCYTGTGCTGACTCA |
| 5′ AgeI Vl 6 | CTGCTACCGGTTCTTGGGCCAATTTTATGCTGACTCAG |
| 5′ AgeI Vl 7/8 | CTGCTACCGGTTCCAATTCYCAGRCTGTGGTGACYCAG |
| 3′ XhoI Cl | CTCCTCACTCGAGGGYGGGAACAGAGTG |

Cloning PCR (SEQ ID NOs. 32-61)

| Primer | Sequence |
|---|---|
| 5′ VH1/5/7 | ATCCTTTTTCTAGTAGCAACTGCAACCGGTGTACATTCCGAGGTGCA |
| | GCTGGTGCAG |
| 5′ VH3 | ATCCTTTTTCTAGTAGCAACTGCAACCGGTGTACATTCTGAGGTGCA |
| | GCTGGTGGAG |
| 5′ VH3-23 | ATCCTTTTTCTAGTAGCAACTGCAACCGGTGTACATTCTGAGGTGCA |
| | GCTGTTGGAG |
| 5′ VH4 | ATCCTTTTTCTAGTAGCAACTGCAACCGGTGTACATTCCCAGGTGCA |
| | GCTGCAGGAG |
| 5′ VH4-34 | ATCCTTTTTCTAGTAGCAACTGCAACCGGTGTACATTCCCAGGTGCA |
| | GCTACAGCAGTG |
| 5′ VH3-9/3′/33 | ATCCTTTTTCTAGTAGCAACTGCAACCGGTGTACATTCTGAAGTGCA |
| | GCTGGTGGAG |
| 5′ VH6-1 | ATCCTTTTTCTAGTAGCAACTGCAACCGGTGTACATTCCCAGGTACA |
| | GCTGCAGCAG |
| 5′ VK1 | ATCCTTTTTCTAGTAGCAACTGCAACCGGTGTACATTCTGACATCCA |
| | GATGACCCAGTC |
| 5′ VK1-9/1-13 | ATCCTTTTTCTAGTAGCAACTGCAACCGGTGTACATTCAGACATCCA |
| | GTTGACCCAGTCT |
| 5′ VK1D-43/1-8 | ATCCTTTTTCTAGTAGCAACTGCAACCGGTGTACATTGTGCCATCCG |
| | GATGACCCAGTC |
| 5′ VK2 | ATCCTTTTTCTAGTAGCAACTGCAACCGGTGTACATGGGGATATTGT |
| | GATGACCCAGAC |
| 5′ VK2-28/2-30 | ATCCTTTTTCTAGTAGCAACTGCAACCGGTGTACATGGGGATATTGT |
| | GATGACTCAGTC |
| 5′ VK3-11/3D-11 | ATCCTTTTTCTAGTAGCAACTGCAACCGGTGTACATTCAGAAATTGT |
| | GTTGACACAGTC |
| 5′ VK3-15/3D-15 | ATCCTTTTTCTAGTAGCAACTGCAACCGGTGTACATTCAGAAATAGT |
| | GATGACGCAGTC |
| 5′ VK3-20/3D-20 | ATCCTTTTTCTAGTAGCAACTGCAACCGGTGTACATTCAGAAATTGT |
| | GTTGACGCAGTCT |
| 5′ VK4-1 | ATCCTTTTTCTAGTAGCAACTGCAACCGGTGTACATTCGGACATCGT |
| | GATGACCCAGTC |
| 5′ VL1 | ATCCTTTTTCTAGTAGCAACTGCAACCGGTTCCTGGGCCCAGTCTGT |
| | GCTGACKCAG |
| 5′ VL2 | ATCCTTTTTCTAGTAGCAACTGCAACCGGTTCCTGGGCCCAGTCTGC |
| | CCTGACTCAG |
| 5′ VL3 | ATCCTTTTTCTAGTAGCAACTGCAACCGGTTCTGTGACCTCCTATGA |
| | GCTGACWCAG |
| 5′ VL4/5 | ATCCTTTTTCTAGTAGCAACTGCAACCGGTTCTCTCTCSCAGCYTGT |
| | GCTGACTCA |
| 5′ VL6 | ATCCTTTTTCTAGTAGCAACTGCAACCGGTTCTTGGGCCAATTTTAT |
| | GCTGACTCAG |
| 5′ VL7/8 | ATCCTTTTTCTAGTAGCAACTGCAACCGGTTCCAATTCYCAGRCTGT |
| | GGTGACYCAG |
| 3′ JH1/2 | GGAAGACCGATGGGCCCTTGGTCGACGCCTGAGGAGACGGTGACC |
| | AG |
| 3′ JH4/5 | GGAAGACCGATGGGCCCTTGGTCGACGCTGAGGAGACGGTGACCA |
| | G |
| 3′ JH3 | GGAAGACCGATGGGCCCTTGGTCGACGCTGAAGAGACGGTGACCA |
| | TTG |
| 3′ JH6 | GGAAGACCGATGGGCCCTTGGTCGACGCTGAGGAGACGGTGACCG |
| | TG |
| 3′ JK1/2/4 | AAGACAGATGGTGCAGCCACCGTACGTTTGATYTCCACCTTGGTC |
| 3′ JK3 | AAGACAGATGGTGCAGCCACCGTACGTTTGATATCCACTTTGGTC |
| 3′ JK5 | AAGACAGATGGTGCAGCCACCGTACGTTTAATCTCCAGTCGTGTC |
| 3′ CL | TGTTGGCTTGAAGCTCCTCACTCGAGGGYGGGAACAGAGTG |

TABLE 3
Monoclonal antibody sequences

| mAb | HC Sequence AA | LC Sequence AA |
|---|---|---|
| PIV3HN-03 | QVQLQEWGPGVVKPSQTLSLTCTVS | DIQMXXXPVSLSASVGDRVTITCQA |
| | GGSISSGFYYWRWIRQPAGKGLEWIG | SQDISNYLNWYQQKPGKAPKLLIYD |
| | RLYTSGSTNYHPSFKSGVTISVDTSKN | ASNLETGVPSRFSGSGSGTDFTFTI |
| | QFSLKMRCVTAADTAVYYCARSKGYS | SSLQPEDIATYYCQHFDNVPMYTF |
| | NDYLKYXXYGMDVWGQGTTVTVSS | GQGTKLEIT |
| PIV3HN-04 | EVQLVQSGAEMKKPGASVKVSCKAS | DIQMTQSPSFLSASVGDRVTITCRA |
| | GYTYTNYYIHWVRQAPGLGLEWMGIV | SQGISTYLAWYQQKPGKAPKLLIYG |
| | NPTGDGTSYAPKFKGRVTMTGDTSTS | TSTLQSGVPSRFSGTGSGTEFILTIS |
| | TVYMELNNLKSDDTAVYYCARARVNG | SLQPEDFATYYCQQLDSYPVTFGG |
| | VFESDYWGPGTLVSVSS | GTRVEIK |
| PIV3HN-05 | EVQLVESGGGVVQPGGSLRLSCVAS | QSVLTQPPSASGTPGQGVTISCSG |
| | GLSLSPNWMHWVRQAPGKGLVWVS | SNSNIGTNAVDWYQQFPGTAPRLLI |
| | RISDNGDTTNYAGSVMGRFTISRDNA | FNDNQRPSGVPDRFSGSRSGTSA |
| | KNTLYLQMNNLRADDTAVYFCVRESA | SLAISGLQSEDEAVYYCATWDDSL |
| | VISSPSWGLYDTAYHFDPWGQGTLVT | NGPVVFGGGTKLTVL |
| | VSS | |
| PIV3HN-09 | QVQLQESGPGLVKPSETLSLTCTVSG | EIVLTQSPATLSLSPGERATLSCRA |
| | GSVSSGSYYWSWIRQPPGKGLEWIG | SQSVSTYLAWYQQKPGQAPRLLM |
| | DIFTSGSTSYSPSLKSRVTISLDGSKN | YDASNRATGIPARFSGSGSGTDFTL |
| | HFSLKLRSVSSADAAVYYCARESWEG | TISSLESEDFAVYYCQQRSIRPWTF |
| | AYLKYFDYWGQGTLVTVSS | GQGTKVEIK |
| PIV3HN-10 | QVQLQESGPGLVKPSETLSLTCTVSG | EIVLTQSPATLSLSPGERATLSCRA |
| | GSVSSGSYYWSWIRQPPGKGLEWIG | SQSVSTYLAWYQQKPGQAPRLLM |
| | DIFTSGSTSYSPSLKSRVTISLDGSKN | YDASNRATGIPARFSGSGSGTDFTL |
| | HFSLKLRSVSSADAAVYYCARESWEG | TISSLESEDFAVYYCQQRSIRPWTF |
| | AYLKYFDYWGQGTLVTVSS | GQGTKVEIK |
| PIV3HN-11 | EVQLVQSGAEVKEPGASVKVSCKAS | EIVLTQSPGTLSLSPGERATLSCRA |
| | GYISTDYYIHWVRQAPGQGPEWMAIL | SQSVGSSYLAWYQQKPGQTPRLLI |
| | NPGGGSPSYAQRFQDRVSVTRDTST | YGASNRATGIPDRFSGSGSGTDFT |
| | ETVYMELSRLRPEDTAVYYCVRDQYD | LTISGLEPEDFAVYYCQHYGRSPLS |
| | FWTSYYTGVEGRHYYYGLDVWGQGT | STFGQGTKVEIK |
| | TVTVSS | |
| PIV3HN-12 | QVQLQESGPGLVKPSETLSLTCTVSG | EIVLTQSPATLSLSPGERATLSCRA |
| | GSVSSGSYYWSWIRQPPGKGLEWIG | SQSVSTYLAWYQQKPGQAPRLLM |
| | DIFTSGSTSYSPSLKSRVTISLDGSKN | YDASNRATGIPARFSGSGSGTDFTL |
| | HFSLKLRSVSSADAAVYYCARESWEG | TISSLESEDFAVYYCQQRSIRPWTF |
| | AYLKYFDYWGQGTLVTVSS | GQGTKVEIK |
| PIV3HN-13 | EVQLLESGGALVQPGGSLRVSCAASG | DIVMTQSPLSLPVTPGEPASISCRS |
| | FSFSSYAMSWLRQTPGKGLEWVSAIG | SQSLRHSDGNNYLDWYLQKPGQS |
| | GSGHSTYYADSVQGRFTVSRDNSKD | PQLLIYLGSNRASGVPDRFSGSGS |
| | TLYLQMNSLRAEDTAVYYCAKFFRSD | GSDFTLKISRVEAEDVGVYYCMQA |
| | GVFHFDYWGPGNPGSPSP | LQTPTFGQGTKVEIK |
| PIV3HN-15 | QVQLQESGPGLVKPSETLSLTCTVSG | SYELTQPPSVSVAPGKTARITCGGN |
| | GSISSYYWSWIRQPAGKGLEWIGRIY | NIGSKSVHWYQQKPGQAPVLVIYY |
| | SSGSTKYSPSLRGRVTMSLDRSKNQF | DSDRPSGIPERFSGSNSGNTATLTI |
| | SLKLTSVSAADTAVYYCARDRFIAVAA | SRVEAGDEADYYCQVWDSSSDHP |
| | DTRLGMDVWGQGTTVTVSS | VFGGGTKLTVL |

HC amino acid sequences in Table 3 correspond to SEQ ID NOs. 62-70, respectively, while HC DNA sequences encoding the amino acid sequences correspond to SEQ ID NOs. 71-79, respectively. LC amino acid sequences in Table 3 correspond to SEQ ID NOs. 80-88, respectively, while LC DNA sequences encoding the amino acid sequences correspond to SEQ ID NOs. 89-97, respectively.

To confirm the enrichment strategy of binding to soluble HN domains selected for antibodies capable of binding to full length HN protein, binding of two mAbs, PIV3HN-05 and PIV3HN-09, to PIV3-infected cells was assessed using flow cytometry. Following a 48-hr infection of LLC-MK2 cells with PIV3, cells were stained with phycoerythrin (PE)-conjugated mAbs, which were tested alongside the anti-PIV3 F positive control mAb PIA174 and the anti-human metapneumovirus F negative control mAb MPV467. mAbs PIV3HN-05, PIV3HN-09, and PIA174 stained the infected cells while mAb MPV467 did not (FIG. 1D). This supported the hypothesis that mAbs targeting HN head domains retain the ability to bind to full-length HN and can identify PIV3-infected cells similarly to mAbs targeting F.

Three Distinct Binding Epitopes were Discovered on the PIV3 HN Protein.

The binding epitope groups of the mAbs on the head domain of the PIV3 HN protein were next determined using competitive biolayer interferometry (BLI) (FIG. 1E). Hexahistidine-tagged PIV3 HN was loaded onto an anti-HIS sensor prior to associating with one mAb. Following an increase in binding, the sensor was associated with a second mAb and competition for the same epitope was identified from the lack of binding after the second mAb association. Three distinct epitopes on the PIV3 HN protein were mapped using this method, termed Sites 1-3 (FIG. 1F). Site 2 and Site 3 contained two mAbs that cross-competed, PIV3HN-03 and PIV3HN-04, suggesting these two epitopes are in proximity on the PIV3 HN protein. Site 1 contained the two least potent neutralizing mAbs, PIV3HN-12 and PIV3HN-13, while Site 2 contained the three most potent neutralizing mAbs, PIV3HN-03, PIV3HN-05, and PIV3HN-11, suggesting that Site 2 plays an important role in viral infection and replication. No correlations were found between epitope and V(D)J gene usage of mAbs (Tables 4-5).

TABLE 4
V(D)J gene usage

| mAb | Gene | Heavy Chain | Light Chain |
|---|---|---|---|
| PIV3HN-03 | V | IGHV4-61*02 | IGKV1-33*01 |
| | D | IGHD5-18*01 | — |
| | J | IGHJ6*02 | IGKJ2*01 |
| PIV3HN-04 | V | IGHV1-46*01 | IGKV1-9*01 |
| | D | IGHD2-8*01 | — |
| | J | IGHJ4*02 | IGKJ4*01 |
| PIV3HN-05 | V | IGHV3-74*01 | IGLV1-44*01 |
| | D | IGHD6-6*01 | — |
| | J | IGHJ5*02 | IGLJ2*01 |
| PIV3HN-09 | V | IGHV4-61*03 | IGKV3-11*01 |
| | D | IGHD5-24*01 | — |
| | J | IGHJ4*02 | IGKJ1*01 |
| PIV3HN-11 | V | IGHV1-46*01 | IGKV3-20*01 |
| | D | IGHD3-3*01 | — |
| | J | IGHJ6*02 | IGKJ1*01 |
| PIV3HN-12 | V | IGHV1-69*01 | IGLV3-21*04 |
| | D | IGHD1-26*01 | — |
| | J | IGHJ4*02 | IGLJ3*02 |
| PIV3HN-13 | V | IGHV3-23*04 | IGKV2-28*01 |
| | D | IGHD2-8*01 | — |
| | J | IGHJ4*02 | IGKJ2*01 |
| PIV3HN-15 | V | IGHV4-4*07 | IGKV1-5*03 |
| | D | IGHD6-16*01 | — |
| | J | IGHJ6*02 | IGKJ2*02 |

TABLE 5
CDR lengths and junctions

| | | CDR-IMGT | |
|---|---|---|---|
| mAb | Chain | length | CDR3 sequence (SEQ ID NOs. 98-113) |
| PIV3HN-03 | HC | [10.7.20] | CARSKGYSNDYLKYXXYGMDVW |
| | LC | [6.3.10] | CQHFDNVPMYTF |
| PIV3HN-04 | HC | [8.8.13] | CARARVNGVFESDYW |
| | LC | [6.3.9] | CQQLDSYPVTF |
| PIV3HN-05 | HC | [8.8.23] | CVRESAVISSPSWGLYDTAYHFDPW |
| | LC | [8.3.12] | CATWDDSLNGPVVF |
| PIV3HN-09 | HC | [10.7.15] | CARESWEGAYLKYFDFW |
| | LC | [6.3.9] | CQQRSIRPWTF |
| PIV3HN-11 | HC | [8.8.26] | CVRDQYDFWTSYYTGVEGRHYYYGLDVW |
| | LC | [7.3.11] | CQHYGRSPLSSTF |
| PIV3HN-12 | HC | [8.8.15] | CARGLSRYTRELLFHYW |
| | LC | [6.3.11] | CQVWDSSSDHPVF |
| PIV3HN-13 | HC | [8.8.14] | CAKFFRSDGVFHFDYW |
| | LC | [11.3.8] | CMQALQTPTF |
| PIV3HN-15 | HC | [8.7.18] | CVKGRYTIADYFEHW |
| | LC | [6.3.9] | CQQNYDLPFTF |

PIV3 HN mAbs Targeting all Three Epitopes can Inhibit Fusion.

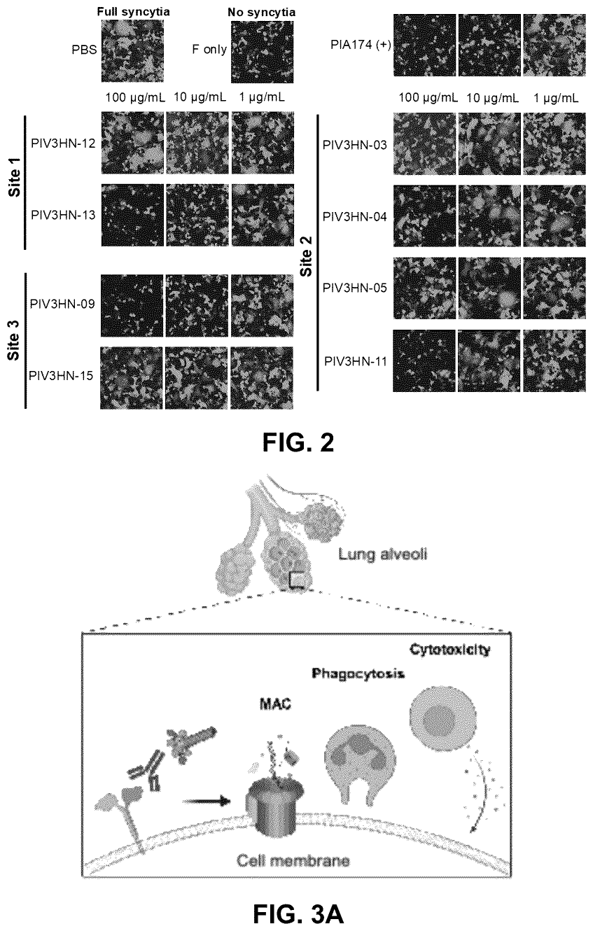

The PIV3 HN protein has several proposed functions, one of which is activating the F protein to initiate a conformational change from the pre-fusion to the post-fusion conformation after binding to sialic acid. To determine the fusion-inhibiting activity of each mAb with respect to the three epitopes on the PIV3 HN protein, independent from receptor binding, Vero-SLAM cells were transfected with plasmids encoding the PIV3 F and PIV3 HN proteins along with green fluorescent protein (GFP). After treating with individual mAbs at decreasing concentrations, syncytia were visualized under a fluorescent cell imaging microscope. mAb PIA174 was used as a positive control since this mAb targets the PIV3 F protein, binds at the apex of the protein, and has been shown to inhibit fusion. Half of the mAbs, including PIV3HN-04, PIV3HN-09, PIV3HN-11, and PIV3HN-13, successfully prevented syncytia formation at the highest concentration of 100 μg/mL, with several inhibiting fusion at a lower concentration of 10 μg/mL (FIG. 2). Fusion inhibition was not site-specific, but rather mAbs in each epitope group were able to inhibit fusion, while others in the same groups had no effect on syncytia formation. Additionally, the fusion inhibition activity was not correlated to the neutralization potency of the mAbs (FIGS. 1B-1C).

mAbs Binding to Site 2 on the PIV3 HN Protein Induce Complement Deposition.

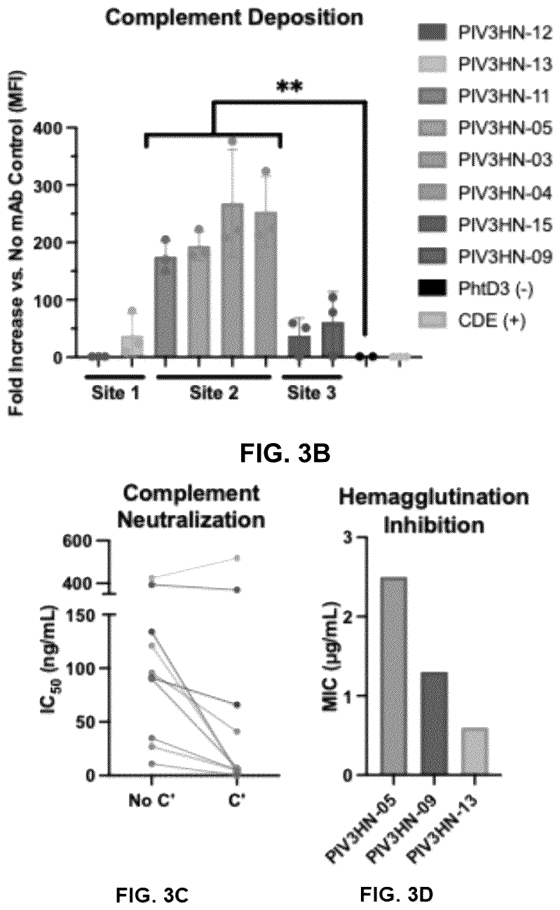

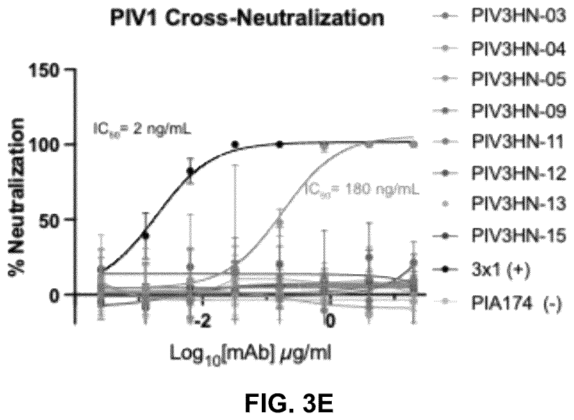

Although mAbs can prevent virus from functioning by binding to a functional epitope via the fragment antigen binding (Fab) region, they can also recruit complement proteins in circulation to bind to the Fc region via the classical pathway. Complement deposition can lead to the formation of the membrane attack complex or can recruit innate cells to kill the infected cell through phagocytosis or cytotoxicity (FIG. 3A). To elucidate the efficacy of mAbs to each site in deposing complement, PIV3 HN-conjugated fluorospheres were stained with individual mAbs complexed with the guinea pig complement C3b protein and detected C3b signal using flow cytometry as previously reported. Beads that had high levels of fluorescence indicated high levels of complement deposition when contrasted to the complement-only control. mAbs binding to Site 2 were highly effective in complement deposition compared to mAbs binding to Sites 1 and 3 (FIG. 3B). To corroborate these experiments, it was sought to determine the neutralizing activity of each mAb in the presence of complement, as the previous neutralization assays were conducted without added complement. As expected, decreases in IC₅₀ values were detected for mAbs binding to Site 2 and Site 3, but not those binding to Site 1 (FIG. 3C).

mAbs from all Sites Inhibit Hemagglutination of Guinea Pig Red Blood Cells.

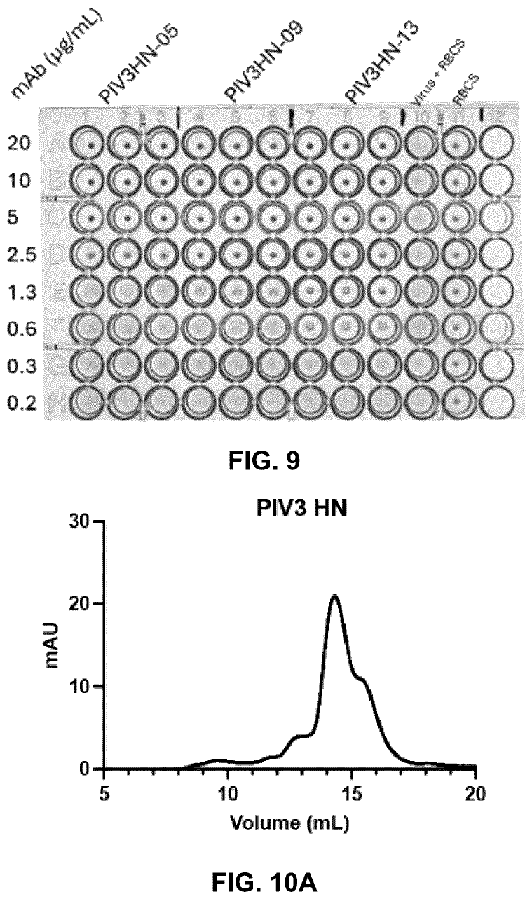

To determine if the mAbs can inhibit functional activity of the PIV3 HN protein, a hemagglutination inhibition (HI) assay was performed using guinea pig red blood cells (RBCs) by co-incubating serial dilutions of mAbs targeting each epitope (PIV3HN-05, PIV3HN-09, and PIV3HN-13) with 4 HA units of PIV3 before adding to guinea pig RBCs. After a 1-2 hour incubation, the minimal inhibitory concentration (MIC) of each mAb to inhibit hemagglutination activity was measured by the last dilution of mAb that displayed HI activity. All three mAbs displayed the ability to inhibit the HA activity of the PIV3 HN protein, suggesting mAbs targeting multiple epitopes of the PIV3 HN protein are able to prevent virus attachment (FIGS. 3D and 9).

### The Site 2-Specific mAb PIVHN-05 Cross-Neutralizes PIV1.

Parainfluenza virus type 1 (PIV1) commonly causes croup in young children and is the second most prevalent circulating parainfluenza virus after PIV3. The two viral seasons occur separately and encompass the calendar year, necessitating a need for a viral therapy that can target both viruses. The HN protein from PIV3 and PIV1 share 49% sequence homology. One PIV1/3 cross-neutralizing mAb, 3′1, has been identified and targets the F protein; however, given the similarity between the two PIV HN proteins, it was sought to identify anti-HN mAbs that can cross-neutralize PIV1 and PIV3 viruses. The neutralizing activity of all eight mAbs against PIV1 was assessed, and only mAb PIVHN-05 successfully neutralized PIV1 with an IC₅₀ value of 180 ng/mL (FIG. 3D).

### Site 1 mAb PIV3HN-13 Binds Near the Dimer Interface of the PIV3 HN Protein.

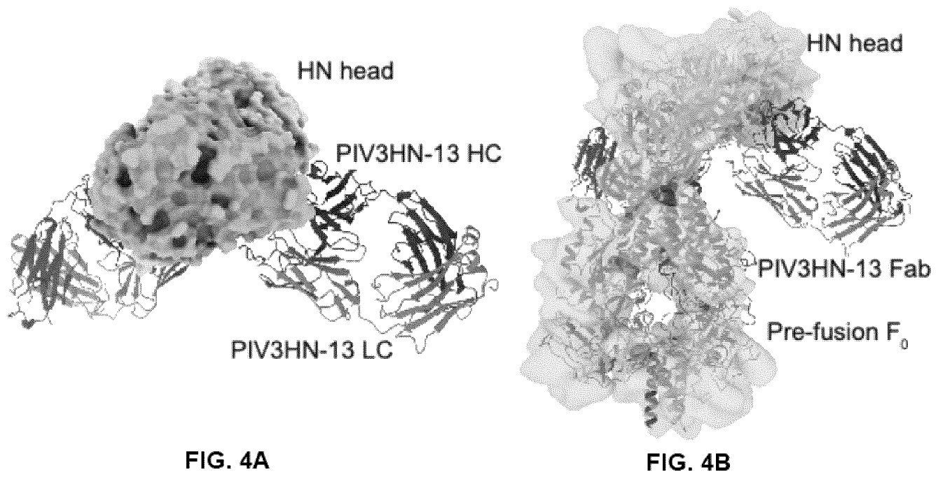

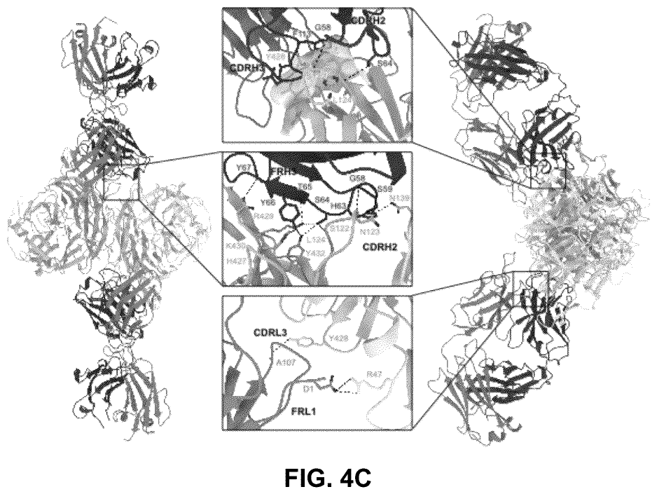

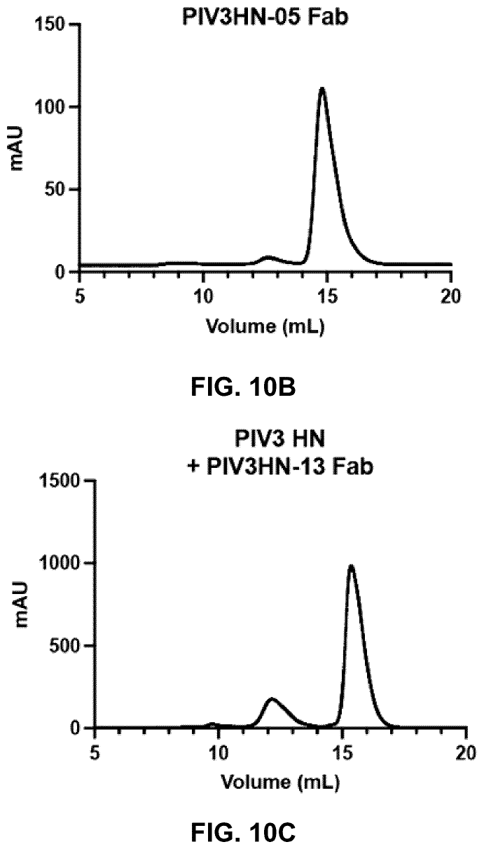

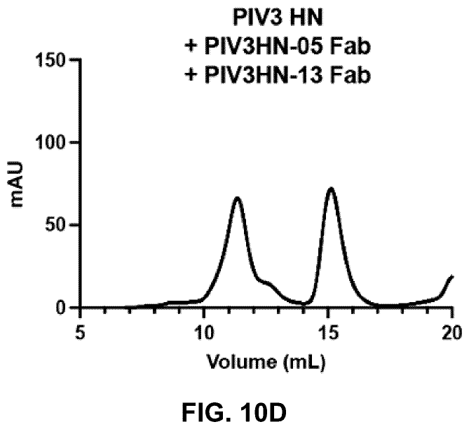

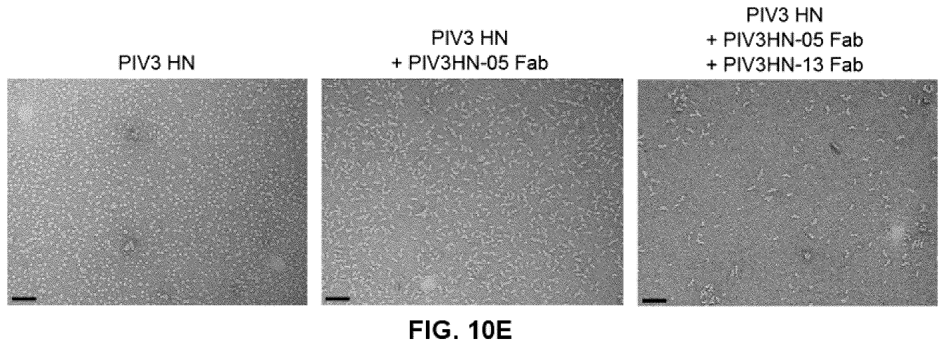

Based on the studies above, the Site 2-targeting mAb PIV3HN-05 was determined to be highly neutralizing (IC₅₀<50 ng/mL), induces complement deposition, and cross-neutralizes PIV1. In contrast, mAbs binding Site 1 (PIV3HN-12 and PIV3HN-13) are weakly neutralizing (IC₅₀>300 ng/mL), inhibit fusion, and lack complement deposition activity. While these two epitopes are discrete on the PIV3 HN protein (FIG. 1F), it was sought to further elucidate these epitopes. For this purpose, structures of one mAb were determined from two epitopes, PIV3HN-13 targeting Site 1 and PIV3HN-05 targeting Site 2. An X-ray crystal structure of the Site 1-binding mAb PIV3HN-13 was determined to 2.4 Å by complexing the PIV3 HN protein and PIV3HN-13 Fab together and confirming successful complexation through size exclusion chromatography (SEC) and negative stain electron microscopy (nsEM) (FIGS. 10A-10E). It was found that mAb PIV3HN-13 binds at the PIV3 HN dimer interface (FIG. 4A, Table 6). There appears to be no steric interactions between the PIV3HN-13 Fab and the PIV3 HN sialic acid active site, found at amino acids 275 to 280 on the H N head domain (FIG. 4A). When fit into a map of the previously proposed PIV3 F-HN interaction (EMD-27550), which occurs shortly after HN activation at the cell membrane, mAb PIV3HN-13 appears to bind distal to the interaction site (FIG. 4B). Interestingly, mAb PIV3HN-13 still inhibited cell-cell fusion, which may be attributed to IgG flexibility or other mAb recruitment functions. The specific interactions found between the PIV3HN-13 Fab and PIV3 HN were assessed and found numerous hydrogen bonds and a hydrophobic pocket between the Fab and antigen (FIG. 4C). Within the heavy chain complementarity determining region 3 (CDRH3), a hydrophobic pocket is formed with three beta hairpins on PIV3 HN (FIG. 4C, top). Surrounded by hydrogen bonds within the CDRH2 and CDRH3, this interface likely creates a hydrophobic attraction to the Site 1 epitope. Along with CDRH2 hydrogen bonds, which are detailed below, one hydrogen bond can be found in the CDRH3 between PIV3HN-13 Fab residue F113 and PIV3 HN residue Y428 (3.0 Å). This hydrophobic pocket possibly strengthens the antibody-antigen interaction and stabilizes the HN protein, which may inhibit further processes such as PIV3 F protein activation. CDRH2 and heavy chain framework region 3 (FRH3) contain the majority of the hydrogen bonds between PIV3HN-13 and PIV3 HN (FIG. 4C, middle). Several hydrogen bonds are present between the CDRH2 and the beta hairpin on PIV3 HN, including Fab to HN residues G58 to S122 (3.1 Å), and S64 to L124 (2.7 Å), respectively. Additionally, S64 on the CDRH2 forms two additional hydrogen bonds with a second beta sheet on HN at residues K430 and Y432 (3.1 Å and 3.1 Å, respectively). Another residue on the CDRH2, S59, forms separate hydrogen bonds with residue N123 on HN, bonding with both the amine and the hydroxyl atoms (2.8 Å and 3.3 Å, respectively). CDRH2 residue T65 forms a hydrogen bond with HN residue K430, the second bond to form with this HN residue (2.9 Å). Of note, one hydrogen bond is formed between the CDRH2 region of mAb PIVHN-13 and the second HN monomer, at residues H63 and N139, respectively (3.2 Å). Additionally, FRH3 residue Y66 forms two hydrogen bonds with HN residues H427 and Y432 (2.7 Å and 2.8 Å, respectively). A final FRH3 residue Y67 forms a bond with N429 on HN (2.9 Å). Although few, the light chain of PIV13 also contains several hydrogen bonds with the HN protein, including those in the light chain CDR3 (CDRL3) and the FRL1 (FIG. 4C, bottom). These bonds include Fab residue D1 binding to both amine groups on HN residue R47 (3.4 Å and 3.1 Å, respectively), along with Fab residue A107 binding to HN residue Y428 (2.8 Å). In total 15 hydrogen bonds were observed between the PIV3HN-13 Fab and the PIV3 HN protein, along with the hydrophobic pocket within the CDRH3 and several beta hairpins in PIV3 HN.

TABLE 6
X-ray crystallography data collection and refinement statistics
PIV3 HN + PIV3HN-13 Fab

| Wavelength | 1.0 | |
|---|---|---|
| Resolution range | 34.36-2.51 | (2.6-2.51)^{a} |
| Space group | P 1 21 1 | |
| Unit cell | 81.273 129.353 89.454 90 93.567 90 | |
| Total reflections | 62013 | (3090) |
| Unique reflections | 61196 | (5863) |
| Multiplicity | 98.4 | (97.7) |
| Completeness (%) | 96.95 | (92.64) |
| Mean I/sigma(I) | 3.33 | (17.8) |
| Wilson B-factor | 39.69 | |
| R-meas | 0.159 | (0.481) |
| R-pim | 0.068 | (0.897) |
| CC½ | 0.985 | (0.897) |
| Reflections used in refinement | 61175 | (5862) |
| Reflections used for R-free | 3097 | (300) |
| R-work | 0.2189 | (0.2966) |
| R-free | 0.2644 | (0.3419) |

| | |
|---|---|
| Number of non-hydrogen atoms | 13582 |
| macromolecules | 13342 |
| ligands | 227 |
| solvent | 13 |
| Protein residues | 1731 |
| RMS(bonds) | 0.004 |
| RMS(angles) | 0.70 |
| Ramachandran favored (%) | 96.03 |
| Ramachandran allowed (%) | 3.85 |
| Ramachandran outliers (%) | 0.12 |
| Rotamer outliers (%) | 2.50 |
| Clashscore | 5.86 |
| Average B-factor | 40.34 |
| macromolecules | 39.87 |
| ligands | 68.04 |
| solvent | 35.57 |

^{a}Statistics for the highest-resolution shell are shown in parentheses.

### The Site 2 mAb PIV3HN-05 Binds Toward the Apex of PIV3 HN, Distal to the F-Interaction Sites

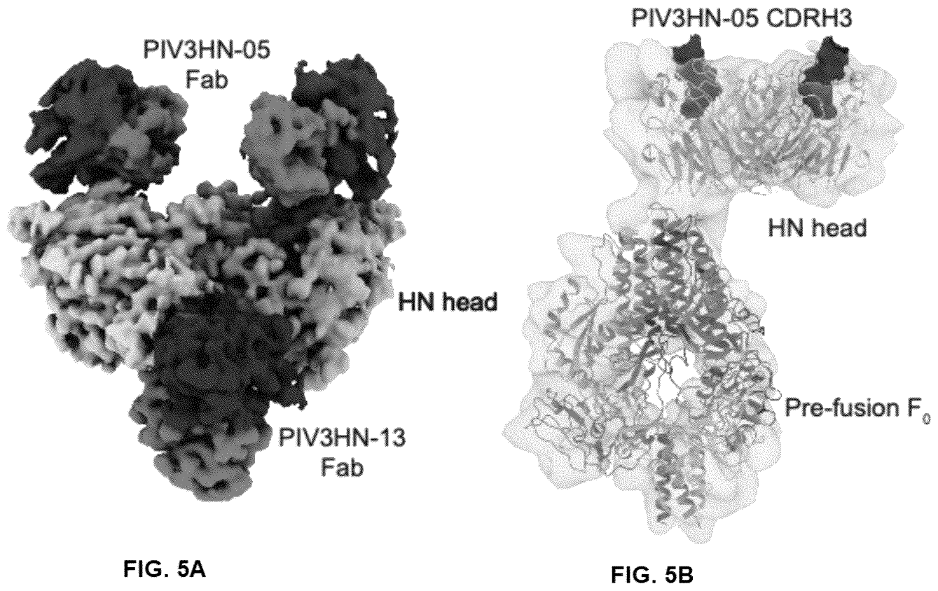

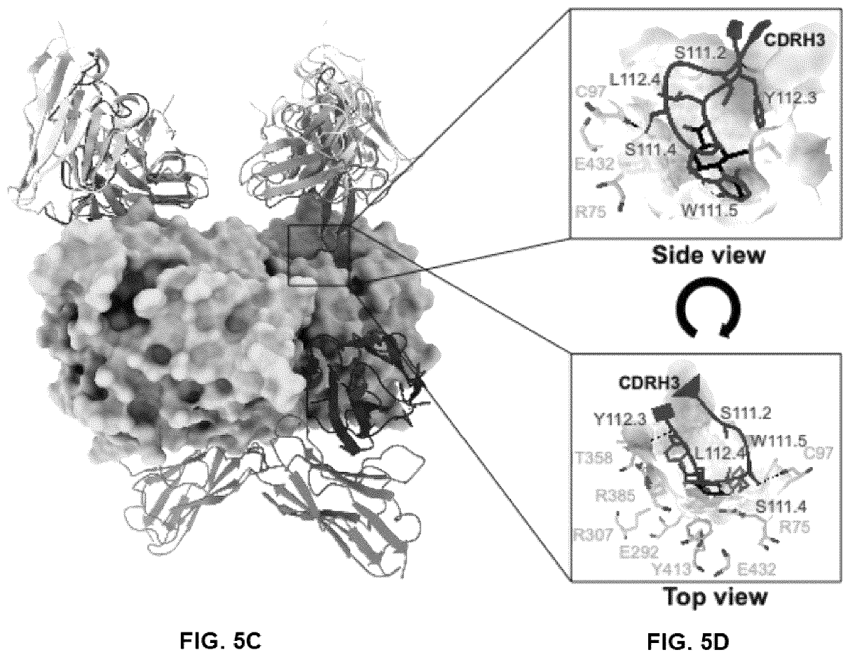

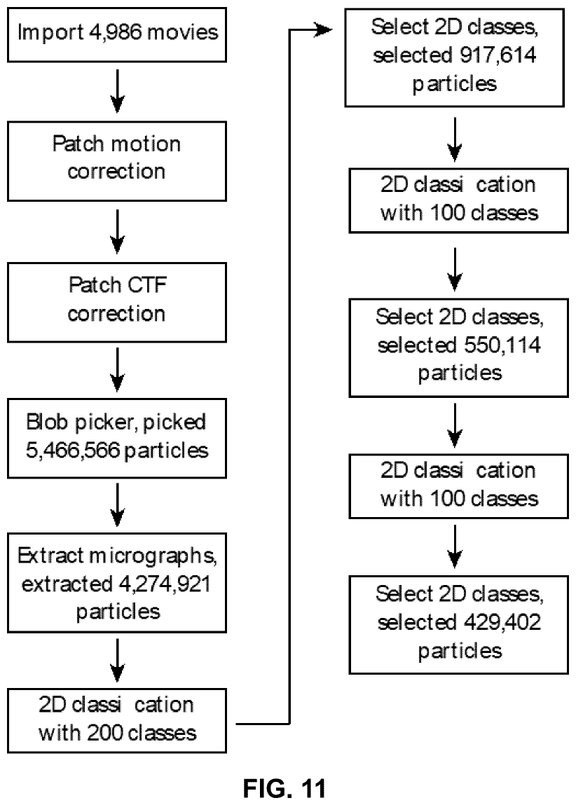

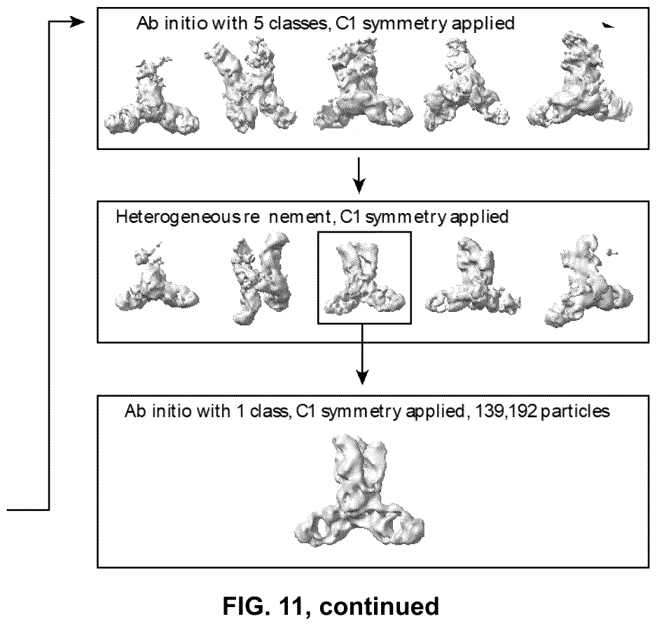

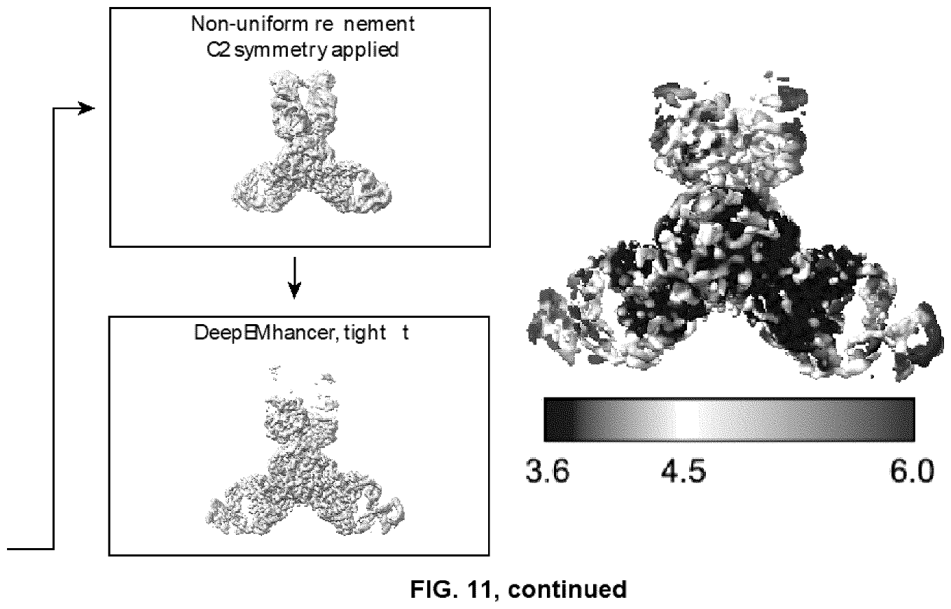

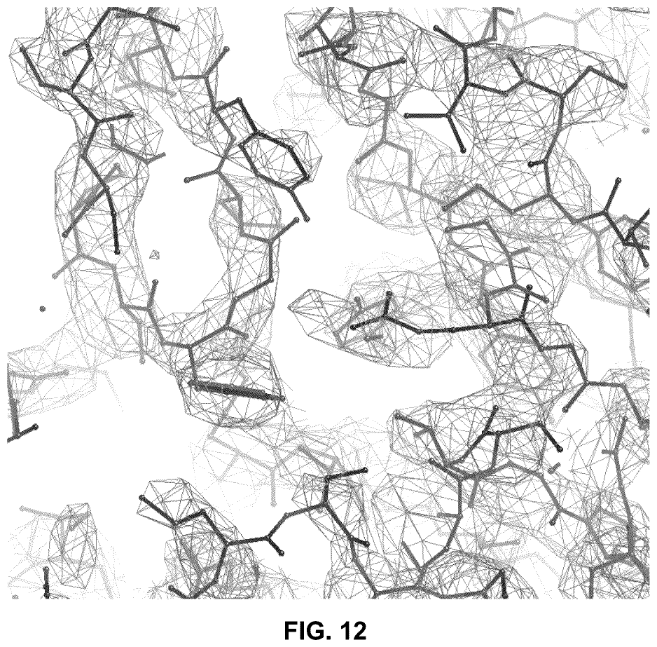

To determine the binding site of mAb PIV3HN-05, cryo-electron microscopy (cryo-EM) was used (FIG. 11). To enable larger particle formation, the discrete binding epitopes, and the X-ray structure determined above, were used to generate a complex containing both Fabs PIV3HN-05 and PIV3HN-13 in complex with the PIV3 HN protein (FIGS. 10A-10E). After particle picking and heterogeneous refinement, an electron density map containing a PIV3 HN dimer with four Fabs bound was obtained and was further processed to a global resolution of 3.57 Å (FIG. 11, Table 7). The density in the map was clear for the PIV3HN-13 Fab and the PIV3 HN protein, however, the density for the PIV3HN-OS Fab contained some disordered regions in portions of the variable region. The PIV3HN-OS Fab binds at the apex of the HN head domain directly within the sialic acid binding site and distal to the binding site of PIV3HN-13, with one Fab binding to one PIV3 HN monomer (FIG. 5A). The CDRH3 of mAb PIV3HN-05 fits deep into the binding pocket of the HN active site and distal to the PIV3 F-HN interaction site, indicating the mechanism of protection by mAb PIV3-05 likely occurs through blocking receptor binding rather than blocking the HN-F protein interaction (FIG. 5B). Importantly, the electron density for the CDRH3 was clearly visible in the cryoEM map allowing a focus solely on the high-resolution interactions between PIV3HN-05 Fab and PIV3 HN. No other interactions besides the CDRH3 were observed with the experimentally built structure. However, since the variable region was missing portions in the electron density, Alphafold3 was used to predict a structure of the PIV3HN-PIV3HN-05 interaction. The Alphafold3 model of PIV3HN-05 Fab with PIV3 HN aligns well with the solved structure of the CDRH3 of the PIV3HN-05 Fab, and also suggested minimal interactions between the PIV3HN-05 Fab and the PIV3 HN protein outside of the CDRH3 (FIG. 5C). Based on the experimentally determined structure, there are two hydrogen bonds between the PIV3HN-05 CDRH3 and the HN active site, respectively: S111D with C97 and Y112C with T358 (both 2.8 Å).

Additionally, hydrophobic interactions are seen within the binding site made by residues on HN previously identified to form the HN active site, seemingly locking the CDRH3 within the active site binding pocket (FIG. 5D). When fit to a previously solved structure of PIV3 HN complexed with a sialic acid analogue, difluorosialic acid (DFSA), the CDRH3 occupies the same space of DFSA, corroborating the hypothesis of PIV3HN-05 blocking sialic acid binding. Like other viral proteins containing hemagglutinin (HA) and neuraminidase (NA) activity, such as influenza virus HA and NA and Newcastle disease virus HN, residues containing sialic acid binding and catalytic activity on PIV3 HN lies at the apex of the head domain. Site 2 was found to be within the sialic acid site of the HN protein, leading to a hypothesis that PIV3HN-05, and other mAbs binding to Site 2, may block receptor binding or prevent viral release by inhibiting HN from interacting with sialic acid.

TABLE 7
Cryo-EM statistics of the PIV3N-
05/PIV3HN-13/PIV3 HN structure
Data collection and processing

| Sample | PIV3 HN + PIV-05 + PIV-13 |
|---|---|
| EMDB ID | EMD-44138 |
| Microscope | Titan Krios |
| Detector | K2 Summit DED |
| Voltage (kV) | 300 kV |
| Electron rate (e−/Å2) | 52.8 |
| Defocus range (μm) | −0.8 to −2.6 |
| Pixel size (Å/pix) | 1.03 |
| Magnification (nominal) | 48,544 |
| Symmetry imposed | C2 |
| Micrographs collected | 4,986 |
| Micrographs used | 4,986 |
| Number of frames | 40 |
| Particles extracted (no.) | 4,256,416 |
| Final particle images (no.) | 139,192 |
| Map resolution (Å) | 3.57 |
| FSC threshold | 0.143 |

Refinement

| | |
|---|---|
| Model composition | |
| Non-hydrogen atoms | 10,459 |
| Protein residues | 1,488 |
| Ligands | 0 |
| B factors (Å²) | |
| Protein (mean) | 56.45 |
| R.m.s. deviations | |
| Bond lengths (Å) (# > 4 σ) | 0.004 |
| Bond angles (°) (# > 4 σ) | 0.760 |
| Validation | |
| MolProbity score | 2.88 |
| Clashscore | 20.11 |
| Rotamer outliers (%) | 0.14 |
| CaBLAM outliers (%) | 3.13 |

### mAb Targeting HN the Active Site can Prevent Viral Replication in Syrian Hamster Lungs Prophylactically and Therapeutically.

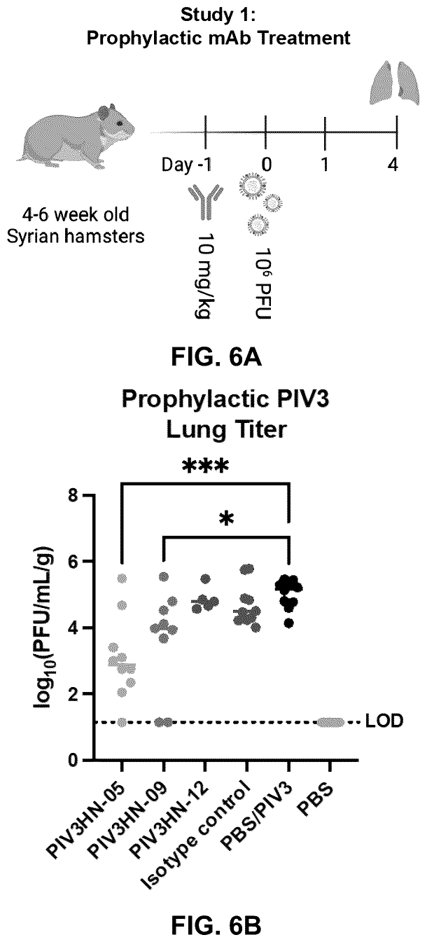

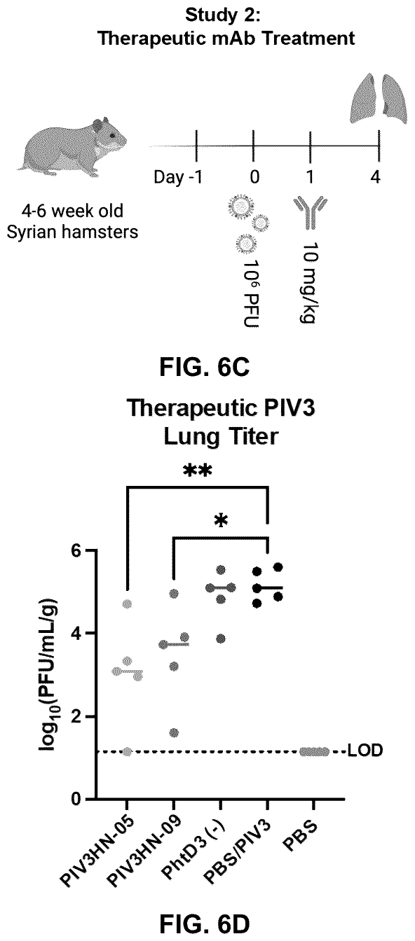

The targeted binding of mAb PIV3HN-05 to the sialic acid binding site on HN, along with its potent neutralization, complement deposition, and PIV1 cross-neutralization, led to the assessment of PIV3HN-05 as a prophylactic and therapeutic treatment in vivo. In addition to PIV3HN-05, the efficacy of mAb PIV3HN-12, which binds to structurally solved Site 1 and inhibits fusion, was tested, as was mAb PIV3HN-09, which binds to Site 3 and is another potent neutralizer of PIV3. For this purpose, the Syrian golden hamster was used, which supports viral replication of PIV3 in the lungs and nasopharynx. In Study 1, hamsters (n=10) were treated intraperitoneally with 10 mg/kg of one mAb to each site 24 hrs before PIV3 viral challenge (FIG. 6A). An isotype control mAb treated group using mAb PhtD7, which targets the pneumococcal histidine triad protein 0, a PBS treated group, and a group of hamsters that were administered PBS as a treatment and as a mock PIV3 infection were also included. Four days after intranasal PIV3 challenge, nasal wash and lung samples were collected and viral load was quantified via plaque assay. Differences in viral load in the lungs were seen in treated hamsters, but viral load in the nasal wash was varied within groups and therefore excluded from statistical comparison.

Compared to the PBS treated and PIV3 infected group (1×10⁵ PFU/mL/g), mAbs PIV3HN-05 (1×10³ PFU/mL/g) and PIV3HN-09 (5×10³ PFU/mL/g), binding to Sites 2 and 3, respectively, effectively decreased PIV3 replication in the lungs, while the Site 1-specific mAb, PIV3HN-12 (7×10⁴ PFU/mL/g), did not (FIG. 6B). The success of the prophylactic mAb treatment of PIV3HN-05 and PIV3HN-09 led to the evaluation of both mAbs as a more clinically relevant therapeutic treatment delivered 24 hrs after PIV3 challenge (FIG. 6C). mAb PIVHN-05 was able to decrease viral load in the lungs (1×10³ PFU/mL/g) compared to the PBS treated group (2×10⁵ PFU/mL/g) (FIG. 6D). mAb PIV3HN-09 was also able to reduce lung viral titers (3×10³ PFU/mL/g) compared to the PBS treated group to a lesser, but still significant, degree (FIG. 6D).

In summary, this paper showcases a specific application of mAb therapy in combating PIV3 infection, specifically with the success of mAb PIV3HN-05 in reducing viral replication in the lungs likely by blocking HN receptor binding. Beyond its immediate impact on PIV3 treatment, the introduction of mAb therapy targeting different activities of HN holds broader implications for the development of treatments against Paramyxoviruses and other respiratory viruses through the assessment of structural targets and mAb functional versatility.

### Example 2: Discussion

Due to the potential for severe PIV3 disease in infants, young children, the elderly, and the immunocompromised, there exists an unfulfilled need for effective vaccines and therapeutics against PIV3. With the recent advancements of other pediatric virus treatments and vaccines, such as for RSV, this paper not only identifies potential mAb treatments to be further assessed for their clinical effectiveness, but also introduces new insights into the PIV3 HN protein and its potential as a vaccine target.

The current understanding of B cell immunity to PIV3 infection is lacking, and these studies help fill gaps in the current knowledge as human mAbs with high levels of potency in both binding affinity and neutralization have been identified. The assessment of mAbs to three discrete epitopes as treatments led to an understanding of the potential of mAbs to Site 2, particularly mAb PIV3HN-05, which was shown to reduce viral replication in the lungs of hamsters. Along with this, the structural interactions between antibody and protein for two mAbs targeting discrete epitopes have been defined.

Of the three identified epitopes, Sites 2 and 3 of PIV3 HN contain the most potent neutralizing mAbs, and mAbs binding to Site 2 are highly effective in complement deposition followed by those binding Site 3. Indeed, the addition of complement increased the neutralizing potency of mAbs binding to Site 2 and Site 3, which may involve Fc-effector functions such as cellular phagocytosis or cellular cytotoxicity. Additionally, while not specific to any binding epitope or correlated to neutralization potency, a subset of mAbs were able to inhibit fusion even at low concentrations, although the mechanism for this inhibition remains to be determined. Of all the mAbs, only the Site 2-specific mAb PIV3HN-05 demonstrated cross-neutralizing activity against PIV1, the second most prevalent of the PIVs. Thus, now mAbs to both PIV3 HN and to PIV3 F have been identified, and there likely remain additional epitopes capable of eliciting cross-reactive mAbs. However, the mechanism by which these mAbs cross-react between the two viruses remains to be determined.

In addition to the efficacy studies, structures of mAbs binding Sites 1 and 2 on the PIV3 HN protein were determined. The Site 1-binding mAb PIV3HN-13 binds at the PIV3 HN dimer interface, with a hydrophobic pocket within the CDRH3 creating an energetically attractive interface for PIV3 HN binding and numerous hydrogen bonds within the CDRH2 likely strengthening the bond between the Fab and antigen. The cryo-EM map and solved CDRH3 structure of the protective mAb PIV3HN-05 identified that Site 2 lies at the apex of PIV3 HN directly inside the sialic acid binding site and distal from both Site 1 and the F interaction site toward the base of the HN head domain. With the promising functional activities of this mAb and the clear resolution of the CDRH3 secured inside the active site of HN, mAb PIV3HN-05 was pursued as a treatment in vivo.

The prophylactic efficacy of mAbs targeting each of the three discovered epitopes was assessed in a whole-body system using a model of PIV3 infection in Syrian golden hamsters. The Site 1 mAb PIV3HN-12 did not influence lung viral titers, while the Site 3 mAb PIV3HN-09 was somewhat effective in Study 1 but not Study 2. However, mAb PIV3HN-05 successfully decreased the lung viral load in hamsters and was effective whether it was administered prophylactically or therapeutically. Based on these data, and the fact that mAb PIV3HN-05 binds to the sialic acid binding site, is potently neutralizing, effective at complement deposition, and cross-neutralizes PIV1, PIV3HN-05 is marked with high potential as a clinical treatment for PIV3 infection and possibly for PIV1 infection. However, the activity of PIV3HN-05 against PIV1 infection in vivo still needs to be assessed in future studies.

Overall, these studies provide new immunological insights into the areas of focus for HN-directed therapies, through way of potential receptor binding inhibition, antibody-mediated immunity, in vivo efficacy, and structural interactions. Additional in vivo experiments can be conducted to study the effectiveness of a mAb cocktail treatment by combining multiple neutralizing mAbs binding to different epitopes and inhibiting various HN functions, testing modifications of Fc domains to increase complement deposition, and determining the efficacy of mAb PIV3HN-05 against PIV1. Structural insights regarding mAbs binding to Site 2 will also need to be further assessed, due to the disordered density of the cryo-EM map generated for the PIV3HN-05 Fab/PIV3 HN structure. Site 3 will need to be further studied to complete the structural assessment of the three epitopes defined against HN, which may lead to future investigations on neutralizing mechanisms of mAbs and their effectiveness in vivo. It is also highly likely additional epitopes on the PIV3 HN protein will be identified as additional human mAbs are isolated. In summary, these data and future data may lead to development of effective vaccines and therapeutics against PIV3 infection and other PIVs, and prompt additional research surrounding the functional aspects of the PIV3 HN protein as well as HN proteins from other PIVs.

### Example 3: Materials and Methods

### Study Design

The overall objective of this study was to identify novel mAbs against the HN protein on PIV3 and assess their neutralizing capabilities, protection in vivo, and structural characteristics. These goals were completed using experiments surrounding binding analysis, virus neutralization, in vivo treatment, and structural analysis, all of which are detailed below in the Materials and Methods. mAbs were labeled as potent binders and neutralizers by EC₅₀ values and IC₅₀ values below 50 ng/mL and 200 ng/mL, respectively.

### Cells and Virus

LLC-MK2 cells were obtained from ATCC (CCL-7) and grown in Opti-MEM (Gibco, Cat #31985070) supplemented with 2% FBS (Gibco, Cat #26140079) at 37° C., 5% CO₂. To split, cells were washed twice with PBS (Corning, Cat #21040CV) and trypsinized with 0.25% trypsin-EDTA (Corning, Cat #25053C1) and neutralized with Opti-MEM, 2% FBS media. PIV3 strain C243 was obtained from IRR (VR-93). PIV1 strain C35 was obtained from IRR (VR-94). Virus was diluted into virus medium (Opti-MEM, 5 μg/mL trypsin-EDTA, and 1% antibiotic-antimycotic) and added to LLC-MK2 cells grown at an MOI of 0.01. Virus was propagated at 37° C., 5% CO₂ for 3-5 days until CPE was observed. Virus was subsequently harvested using a sucrose freeze/thaw method as previously described. The virus-containing sucrose solution was aliquoted, flash-frozen in liquid nitrogen, and stored at −80° C. until further use.

### PIV3 HN Head Protein Construct Design, Expression, and Purification.

A plasmid construct for the head domain of PIV3 HN was designed based on the wild-type PIV3 strain C243 HN sequence (GenBank JN089924.1) with the N-terminal cytoplasmic tail and transmembrane domains removed and a Hexahistidine tag added. Plasmid containing the HN head domain insert was transformed into DH5a E. coli competent cells prior to plasmid isolation and transfection. Recombinant protein was expressed in Expi293F cells (ThermoFisher, Cat #A14527) as previously described. Culture supernatant was filtered through a 0.45 μm filter before purifying through a HisTrap excel pre-packed column (Cytiva, Cat #17371206) per the manufacturer's instructions. Purified protein was buffer exchanged into PBS and stored at −80° C. until use.

Isolation of Peripheral Blood Mononuclear Cells from Healthy Human Donors

Peripheral blood mononuclear cells (PBMCs) from Subject 1 were obtained through study recruitment to the University of Georgia Clinical and Translational Research Unit and written informed consent was obtained. This study was approved by the University of Georgia Institutional Review Board as PROJECT00002304. PBMCs from Subjects 2-3 were obtained from leukocyte reduction filters supplied by Shepheard Community Blood Center in Augusta, Georgia. Each filter was gently washed with PBS and the flow-through, which contains the leukocytes, was collected in a 50 mL conical tube. In a separate tube, 13 mL of warmed Ficoll-Paque (Cytiva, Cat #17144002) was added to a separate 50 mL conical tube. The cells were gently added to the top of the Ficoll layer then centrifuged at 2500 rpm for 30 min at 4° C. with a slow acceleration and deceleration rate. The PBMC layer below the PBS and above the Ficoll was gently aspirated from the tube and added to a separate tube containing Dulbecco's Modified Eagle's Medium (DMEM) (Corning, Cat #10014CV) and centrifuged at 300×g for 5 min. The supernatant was discarded, and the cell pellet was resuspended in 10 mL RBC lysis buffer (155 mM NH₄Cl, 12 mM NaCO₃, 0.1 mM EDTA). The cells were centrifuged at 300×g for 5 min, the supernatant was discarded, and the PBMCs were washed with DMEM. Finally, the cells were resuspended in ClonaCell-HY Medium A (StemCell Cat #03801) supplemented with 10% DMSO, transitioned to −80° C., and stored in LN₂ until further use.

Antigen-Specific Flow Sorting with Memory B Cells Against the PIV3 HN Protein

One day before B cell sorting, gamma-irradiated NIH/3T3 cells (ATCC, CRL-1658) modified to express human CD40L, human interleukin-21 (IL-21), and human B-cell activating factor (BAFF) (a gift from Deepta Battacharya), were diluted in StemCell Medium A (0.5 million cells/mL) and supplemented with the TLR agonist CpG (phosphorothioate-modified oligodeoxynucleotide ZOEZOEZZZZZOEEZOEZZZT; Invitrogen) (6 μg/mL) as previously described. The diluted cells were added to the inner wells of a tissue culture-grade 384 well plates (50 μL/well), excluding the outer two rows on each side, which contained sterile diH₂O (100 μL/well) to prevent evaporation of media containing the sorted B cells. Prior to antigen-specific sorting, PIV3 HN was biotinylated as per manufacturer's instructions (ThermoFisher, Cat #21455). Streptavidin-conjugated PE (Invitrogen, Cat #S866) and streptavidin-conjugated BV605 (BioLegend, Cat #405229) were slowly added to biotinylated HN at a fluorophore to protein molar ratio of 4:1 and stored on ice away from light. PBMCs (1×10⁶ cells) were suspended in FACS buffer (PBS, 2% FBS, 2% goat serum, 0.5 mM EDTA) (1 mL) and Fc-blocked with Human TruStain FcX (BioLegend, Cat #422301) (5 μL/million cells) on ice for 30 min. A staining mixture containing anti-human CD19-APC (BioLegend, Cat #302212), IgM-FITC (Southern Biotech, Cat #2020-02), IgD-FITC (Southern Biotech, Cat #2030-02), GhostDye Red 780 (Tonbo Biosciences, Cat #130865T100) or DAPI (ThermoFisher, Cat #62248) (live/dead stains), BT-SA conjugated PIV3 HN-PE and BT-SA conjugated PIV3 HN-BV605 was prepared and kept on ice away from light. Cells were centrifuged at 400×g for 5 min to pellet the cells and washed with FACS buffer once before resuspending in 30 μL FACS buffer. Single-stain controls were prepared with the cells and the remainder of the cells were added to the staining mixture and incubated on ice for 30 min away from light. Cells were washed once with FACS buffer and resuspended in FACS buffer. Antigen-specific live B cells (CD19⁺, IgM⁻, IgD⁻, PE⁺, BV605⁺) were single-cell sorted into each well containing the 3T3 cell monolayer and incubated for 13 days at 37° C., 5% CO₂. Wells containing B cell clones were fed with Medium A (50 μL/well) 7 days after flow sorting.

### Enzyme-Linked Immunosorbent Assay Against the PIV3 HN Protein

For both PBMC screening following B cell sorting and binding curves for transfected mAbs, 384-well high binding plates (Greiner, Cat #781061) were coated with PIV3 HN and the assay performed as previously described. The OD40s values were measured on a Biotek plate reader using Gen5 3.11 and EC₅₀ values were calculated using GraphPad Prism9.

RNA Extraction and DNA Amplification of RNA from Antigen-Positive B Cells

RNA extraction was performed with the Qiagen RNeasy Micro Kit (Qiagen, Cat #74004) according to the manufacturer's instructions. In brief, B cells from positive wells were isolated by gently scraping and pipetting the base of the well and removing the culture prior to lysis and extraction. RNA samples were immediately translated into DNA or stored at −80° C. until use. RNA was translated and amplified into separate heavy chains and light chains using the OneStep RT-PCR kit according to the manufacturer instructions (Qiagen, Cat #210212). Briefly, RNA was thawed on ice and combined with 5× buffer, dNTP mix, enzyme mix, nuclease free water, and a primer mix for heavy chains, kappa chains, or lambda chains, as previously described. The reaction was performed on a Thermal Cycler (50° C.-30 mins, 95° C.-15 mins, 40 cycles of (94° C.-40 s, 55° C.-60 s, 72° C.-60 s), 72° C.-10 min). The reaction product was amplified in a second PCR. DNA product from the previous PCR was combined with 2× DreamTaq Master Mix (ThermoFisher, Cat #K1081), nuclease-free water, and the heavy chain, kappa chain, or lambda chain primer mix. The reaction was performed on a Thermal Cycler (94° C.-5 min, 40 cycles of (94° C.-30 s, 57° C.-30 s, 72° C.-60 s), 72° C.-10 min). Presence of heavy chain and light chain DNA was confirmed on a 1% agarose gel. PCR products were purified using the E.Z.N.A Cycle Pure Kit (Omega Bio-Tek, Cat #D6492-01) according to the manufacturer's instructions before Sanger sequencing. Finally, after the sequences of each heavy chain and light chain were determined, samples were cloned using the appropriate primer based on the sample's V and J genes. DNA was combined with 2× DreamTaq Master Mix, primer mix, and nuclease-free water as previously described. The reaction was performed using a Thermal Cycler (94° C.-4 min, 40 cycles of (94° C.-30 s, 58° C.-30 s, 72° C.-45 s), 72° C.-8 min). DNA product was confirmed on a 1% agarose gel and purified using the E.Z.N.A Cycle Pure Kit prior to ligating into an expression vector.

### Vector Digestion and Ligation

Expression vectors were digested prior to DNA ligation as previously described. Heavy chain, kappa chain, and light chain vectors were used (AbVec-hlgG1 (FJ475055), AbVec-hlgKappa (FJ475056), and PBR322-based Ig-lambda expression vector (FJ517647), respectfully). Digested vectors were separated on a 1.5% agarose gel (100V—5 min, 75V—75 min) and bands at 6000 bp were cut out and purified using the E.Z.N.A. Gel Extraction Kit (Omega Bio-Tek, Cat #D2500-01) according to the manufacturer's instructions. Heavy chain and light chain DNA inserts were combined with the linearized vector, NEBuilder HiFi DNA Assembly Master Mix (New England BioLabs, Cat #E2621S). Assembled vectors were stored at −20° C. until transformations were performed.

Plasmid Transformation into E. coli Competent Cells

Following vector ligation, assembled vectors were transformed into E. Coli DH5a competent cells as previously described. Colony PCR was performed as described above to confirm successful transformation. Individual colonies were selected for expansion into LB cultures with ampicillin (5 mL) and shaken for 8-12 hrs at 37° C. DNA plasmids were extracted from cultures as described previously. Plasmids were sequenced to confirm mAb transformation, and plasmids were re-transformed into DH5a cells (1 μL) as described above. The bacteria were cultured to a final volume of 250 mL and DNA plasmids were extracted using the E.Z.N.A Plasmid Maxi Kit (Omega Bio-Tek, Cat #D6922-04) according to the manufacturer's instructions. Plasmids were sterile filtered in preparation for mammalian cell transfection.

Plasmid Transfection into HEK-293F and ExpiCHO Cells

mAbs were transfected in either Freestyle293 or ExpiCHO cultures. For HEK293 transfections, volume of mAb plasmids, including both heavy chains and light chains, were calculated based on the final culture volume (1 μg/mL), combined with Opti-MEM media and PEI MAX transfection reagent, and added to Expi293F cells as mentioned above. ExpiCHO transfections were conducted using ExpiCHO-S cells (ThermoFisher, Cat #A29127) the ExpiFectamine CHO transfection kit (ThermoFisher, Cat #A29129) under the Max Titer protocol as described by the manufacturer.

### mAb Purification

Six days after plasmid transfection in HEK293 cells, or 14 days after plasmid transfection in ExpiCHO cells, the culture was centrifuged at 6000×g for 10 min. The supernatant was filtered through a 0.45 μm filter. PBS was run through a HiTrap Protein G prepacked column (Cytiva, Cat #17040503) according to the manufacturer's instructions. Eluted mAbs were buffer exchanged with PBS. Concentrations of each mAb were taken on a Denovix system using the IgG setting. An SDS-PAGE was performed to confirm successful transfection of mAbs under non-reducing and reducing conditions and stained with Coomassie Blue.

### PIV3 mAb Plaque Reduction Neutralization Assay (PRNT)

PIV3 mAbs were serially diluted in Opti-MEM and incubated with PIV3 in a 1:1 ratio for 1 hr at room temperature. The mAb/virus mixture was added to LLC-MK2 (Opti-MEM, 2% FBS) monolayer in a 24-well tissue culture plate (50 μL/well) and rocked for 1 hr at 37° C., 5% CO₂. Cells were overlaid with warm 0.75% methylcellulose in Opti-MEM media supplemented with 5 μg/mL trypsin-EDTA and 1% antibiotic-antimycotic and incubated for 4 days at 37° C., 5% CO₂. Following incubation, cells were fixed with 10% neutral buffered formalin for 30 min at room temperature, washed 3 times with water, and blocked with blocking solution (2% milk blocking solution) supplemented with 2% goat serum in 0.05% PBS-Tween-20 for 1 hr at room temperature. Plates were washed 3 times with water and the primary antibody (PIA174 for PIV3, 3′1 for PIV1) was added (5 μg/mL in blocking buffer, 200 μL/well) for 1 hr at room temperature. Plates were washed 3 times with water and HRP-conjugated secondary antibody (goat anti-human IgG Fc HRP, Southern Biotech Cat #2048-05) was added to the wells at a 1:2000 dilution (200 μL/well) for 1 hr at room temperature. Plates were washed 5 times with water and developed with TrueBlue substrate (SeraCare, Cat #5510-0030) (120 μL/well) and rocked for 10 min at room temperature before washing once with water and allowed to dry. Immunostained plaques were manually counted with a stereomicroscope. IC₅₀ values were calculated using GraphPad Prism9.

Flow Cytometry with PIV3-Infected Cells

Fluorophore-conjugated antibody was prepared by incubating biotinylated mAb with streptavidin-conjugated PE (Invitrogen, Cat #S866) for 1 hour on ice 24 hours prior to cell staining. LLC-MK2 cells were grown in a T75 flask (Opti-MEM, 2% FBS) at 37° C., 5% CO₂ to 80% confluency prior to infection. Cells were washed twice with PBS before infecting with PIV3 (MOI=0.01). Cells were incubated with virus for 1 hour at 37° C., 5% CO₂, rocking every 10 mins. Virus culture media (Opti-MEM, 5 μg/mL trypsin-EDTA, 1% antibiotic-antimycotic) was added to the flask after 1 hour and cells were infected for 48 hours. After infection was complete, cells were digested with Versene (Gibco, Cat #15040-066) at 37° C., 5% CO₂ for 30-45 mins until cells were no longer attached to the flask. Cells were washed twice with FACS and incubated in FACS buffer on ice for 30 mins. Cell aliquots were individually stained with PE-conjugated mAb (PE-PIV05, PE-PIV09, PE-PIA174, PE-MPV467) on ice away from light for 30 mins. Cells were washed twice with FACS buffer and resuspended in FBS. Cells positive for PE were analyzed on the NovoCyte Quanteon Flow Cytometer and analyzed in FlowJo.

### Biolayer Interferometry Competition Assay

PIV3 HN protein monomer and human mAbs were diluted at 100 μg/mL in Octet buffer (PBS, 0.02% Tween-20, 0.1% BSA). After obtaining a baseline in Octet buffer for 60 sec, PIV3 HN was loaded onto HIS1K Biosensors (Sartorius, Cat #18-5120) for 120 sec. mAbs were primarily associated with HN for 300 sec, then the biosensors were placed directly into wells with mAbs for 300 sec for comparative competition. Biosensors were regenerated with glycine (0.01 M, pH 2.7) 5 times for 6 sec before reusing. The degree of competition was calculated by dividing the total binding (nm) of the second mAb by the total binding (nm) of the first mAb×100. Higher values indicate lower competition, whereas lower values indicate high levels of competition.

### Fusion Inhibition Assay

Vero-SLAM cells were transfected with plasmids encoding PIV-HN, PIV-F, and GFP at a 3:3:1 ratio with JetOptimus transfection reagent (PolyPlus, Cat #101000006) as per manufacturer's instructions. Three hours following transfection, the media was replaced, and antibody or PBS was added to the cells at the indicated concentration. Syncytia was visualized 24 hours following transfection and images were taken using a Zoe microscope (Bio-Rad) (20× magnification).

### Hemagglutination Inhibition Assay

For the HAI experiment, 25 μL of 4 HA units of HPIV3 were incubated with 25 μL two-fold serially diluted antibodies in PBS (starting with 20 μg/mL of each antibody) for 1 hr at room temperature. Then, 50 μL of 0.5% guinea pig RBCS (Innovative Research Inc.) was added to the virus-antibody mixture and allowed to settle for 1-2 hrs at room temperature. Minimal inhibitory concentration of each antibody was detected as the lowest concentration that inhibits the HA activity of 4 HA units of PIV3.

### Complement Deposition Flow Cytometric Assay

Antibody dependent complement deposition was performed as previously described. In brief, biotinylated HN antigen was coupled with FluoSpheres NeutrAvidin beads (Invitrogen, Cat #F8776) at a 1:1 ratio of antigen (μg): beads (μL). Beads are then washed twice in 5% PBS-BSA and resuspended at 1:100 of starting bead volume in 0.1% BSA. MAbs were then incubated at 1-10 μg/mL with 10 μL of antigen specific beads for 2 hours at 37° C. Bead-mAb complexes are then washed twice in 0.1% BSA. Guinea pig complement (MP Biomedicals, Cat #8642836) was then diluted in R-10 buffer (RPMI-1640+10% FBS) at a 1:50 dilution. Complement and the bead-mAb complex was incubated for 15 minutes and washed twice in PBS. A 1:100 dilution in PBS of secondary anti-C3 antibody (ICL, Cat #GC3-90P-Z) (fluorescein-conjugated goat anti-guinea pig complement C3) was then incubated at RT for 15 min. Complexes were then washed twice in PBS and resuspended in a final volume of 150 uL PBS. Samples were read on the Novocyte Quanteon.

### Complement PRNT

A PRNT was performed as described above with the addition of guinea pig complement (MP Biomedicals, Cat #8642836). After serially diluting mAb into Opti-MEM, guinea pig complement was added at a 1:1000 volume ratio. PIV3 virus was added to the complement+mAb mixture and the assay was performed as stated above.

### Prophylactic/Therapeutic Treatment and Viral Challenge of Syrian Hamsters

All procedures involving animals were performed in accordance with guidelines of the Institutional Animal Care and Use Committee of the University of Georgia. Four- to six-week-old male and female Syrian hamsters (Charles River Laboratories #049) were single housed in the animal biosafety level 2 (ABSL-2) facility at the University of Georgia College of Veterinary Medicine. Hamsters were given their respective mAb prophylactic treatment (10 mg/kg) through an intraperitoneal (IP) injection one day before viral challenge. 24 hours after mAb prophylaxis, hamsters were anesthetized with 4% isoflurane for 2 min before infecting with PIV3 strain C243 intranasally (IN) (10⁶ PFU/mL, 50 μL). Hamsters were monitored before placing back in cage. Five days after infection, hamsters were humanely euthanized by asphyxiation under CO₂. Lungs were collected and gently washed with sterile PBS, then weighed, added to cold Medium A (3 mL), and homogenized for viral titration. Nasal wash was collected by injecting sterile PBS through the trachea and collecting from the nasal cavity. Unless immediately used, lung and nasal wash samples were stored in 1% SPG buffer at −80° C. following flash freezing in LN₂.

PIV3 Viral Titration from Syrian Hamster Lung and Nasal Wash

Immediately following sample collection, lung homogenate and nasal wash samples were serially diluted in cold Opti-MEM media and added to LLC-MK2 (Opti-MEM, 2% FBS) monolayer in a 24-well tissue culture plate (50 μL/well) and rocked for 1 hour at 37° C., 5% CO₂. The remainder of the plaque assay was performed as described above. Viral titers were calculated as log₁₀(PFU/mL/g) and analyzed in GraphPad Prism9.

### Assembly of Fab-Antigen Complex

Fab was digested using the Pierce Fab Preparation Kit (ThermoFisher, Cat #44985) prior to complexing with HN. Pure Fab and antigen were isolated through size exclusion chromatography (SEC) as described below. Fab and antigen were combined in a 2:1 molar ratio in a low salt buffer (120 mM NaCl, 20 mM Tris, pH 7.5) and incubated overnight at 4° C. The Fab-antigen complex was isolated through SEC prior to use.

### Size Exclusion Chromatography

Individual HN protein, Fab, and Fab-antigen complexes were isolated through SEC on a Superdex S200 10/300 (Cytiva, Cat #28990944) in column buffer (120 mM NaCl, 20 mM Tris, pH 7.5) based on their molecular weight elution profiles. The desired elute was concentrated prior to use.

### PIV3HN-13 Fab:HN Crystal Formation

Complexes were formed by combining a 1:1.2 molar ratio of PIV3HN to Fab. This solution incubated in 4° C. overnight and was then run over a Superdex 200 Increase 10/300 GL column. The peak coordinating with a complex was isolated and concentrated to 9 mg/ml and was then screened using a sitting drop method in which a 0.5 μL protein solution to 0.5 μL crystal condition were combined. Crystals formed between 1-4 weeks. PIV3HN-13 Fab:HN crystallized in a solution of 0.1M Ammonium Sulfate, 0.1M MES pH 6.5, and 15% (w/v) PEG4000 and was flash frozen with no cryo-protectant. PIV3HN-13 Fab crystallized from the Molecular Dimensions 1+2 kit in 0.2 M Ammonium Sulfate, 0.1 M MES pH 6.5, and 30% w/v PEG5000 MME.

### X-Ray Crystallography Data Collection, Processing, and Refinement

Data was collected at the Advanced Photon Source (APS) beamlines 22-ID. Data Processing was performed using HKL-2000 (v 719.2) and CCP4 suite (v 8.0.005). Molecular Replacement was performed Phaser-MR (full-featured) out of the Phenix suite of programs. To phase the complexes, homology models were generated using SWISS-MODEL, PIV3 HN model was based on the previously solved structure (PDB: 1V3B). After phasing structures went through multiple rounds of refinement in Coot (v 0.9.8.3) and Phenix (v1.20.1).

### Negative-Stain Electron Microscopy

The size excluded Fab-antigen complex was diluted into HyPure water (Cytiva, Cat #SH30538.02) to a final concentration of 15 μg/mL. Glow discharged, carbon-coated copper grids were stained with the diluted complex (5 μL) for 3 min, then washed twice with HyPure water. The grid was washed once with 1% uranyl formate solution, then stained in 1% uranyl formate solution for 45 sec. The excess stain was removed with a Kim wipe and the grid air dried. Negative-stain electron micrographs were imaged on the JEOL JEM1011 transmission electron microscope equipped with a high-contrast 2K-by-2K AMT midmount digital camera to obtain 2D images of the Fab-antigen complex before assessing for structural interactions through cryo-electron microscopy.

### Cryo-Electron Microscopy

For Fabs 05+13:HN complex, at a concentration of 1.15 mg/mL, respectively, were applied to Quantifoil 2/1 (300 mesh) grids previously glow-discharged for 15 s at 15 mAmp current on both sides. Grids were blotted for 3 s with 100% humidity and plunge-frozen in liquid ethane using a FEI Vitrobot Mark IV instrument. Cryo-EM data were collected on a Krios G2 (Thermo Fisher) equipped with a K2 summit DED camera (Gatan, Pleasnton, CA.). Cryo-EM movies were acquired using a nominal magnification of 48,544× with a pixel size of 1.03 Å. Movies were recorded as 40-frame videos in counting mode, with a defocus range from −0.8 to −2.6 μm. Data were processed in cryoSPARC according to FIG. 11. Alphafold3 was used to generate an initial model, which was fit into the cryo-EM map in ChimeraX. The model was then manually refined in COOT followed by refinement in Phenix.

### Example 4: HN-Targeted Monoclonal Antibodies Against Human PIV3

### Results

Isolation of PIV3 HN-Specific mAbs

HN-targeted monoclonal antibodies were isolated from healthy donors by sorting peripheral blood B cells for antigen-specific memory B cells. This was done by using fluorescently conjugated HN proteins to identify B cells that recognized the hemagglutinin-neuraminidase surface protein. To obtain the antibody sequences, 10× Genomics sequencing was used to retrieve paired heavy and light chain variable regions from the B cells. The sequences were then cloned into plasmids to generate recombinant monoclonal antibodies. Using this approach, two mAbs were identified and produced that were specific to the HN protein termed 5217-2 and 5217-9. These antibodies were subsequently characterized for their binding properties and protective efficacy and compared to the previously identified HN-specific monoclonal antibodies.

Binding and Neutralizing Properties of the Isolated mAbs

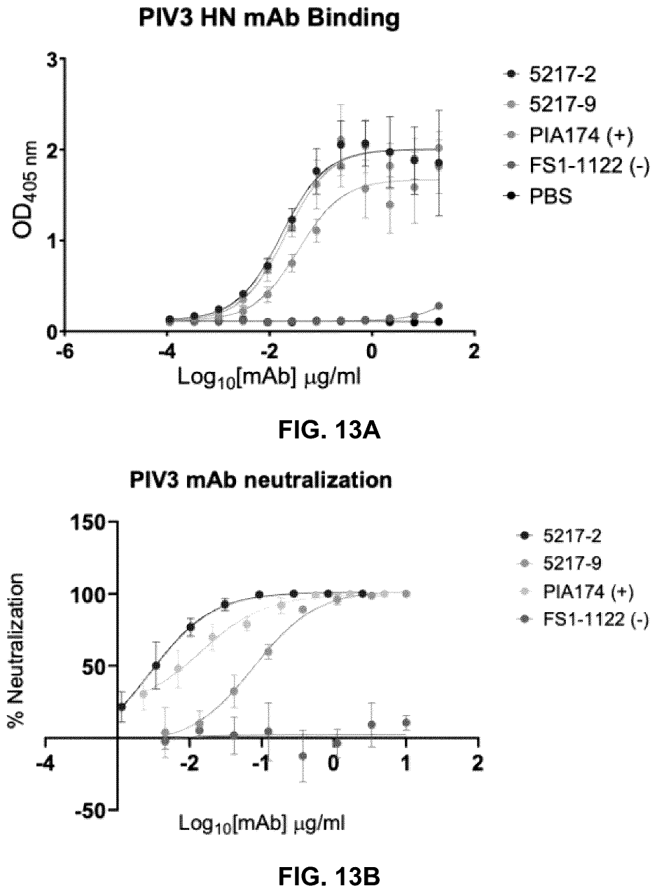

The binding properties of the novel mAbs, 5217-2 and 5217-9, to the PIV3 HN protein were confirmed via enzyme-linked immunosorbent assay (ELISA). Both mAbs bound to the HN protein with an EC₅₀<1.0 μg/mL (FIG. 13A). The mAbs were subsequently tested for their ability to neutralize PIV3 virus in vitro using a plaque reduction neutralization assay (PRNT). Both demonstrated neutralizing activity, with mAb 5217-2 exhibiting the most potent capabilities against PIV3 virus with an IC₅₀ of 3 ng/mL. Notably, mAb 5217-9 displayed reduced neutralizing activity when compared to the positive control, PIA174, with an IC₅₀ of 70 ng/mL (FIG. 13B).

To confirm that these mAbs recognize the native full-length HN protein on the virion surface, their binding to PIV3-infected LLC-MK2 cells was evaluated using flow cytometry. Cells were infected with PIV3 for 48 hours, harvested, and incubated with phycoerythrin (PE)-conjugated mAbs. The novel mAbs were evaluated alongside the established positive control PIA174 and the negative control PhtD3. Consistent with expectations, mAbs 5217-2, 5217-9, and PIA174 stained the cells indicating recognition of the HN protein in its native conformation on the viral surface. In contrast, the negative control mAb, PhtD3, did not show detectable binding. This supports the functional relevance of the epitope targeted by 5217-2 and 5217-9, as it is accessible and conserved in the context of infection.

### Epitope Mapping Reveals a Novel Binding Site on the PIV3 HN Protein

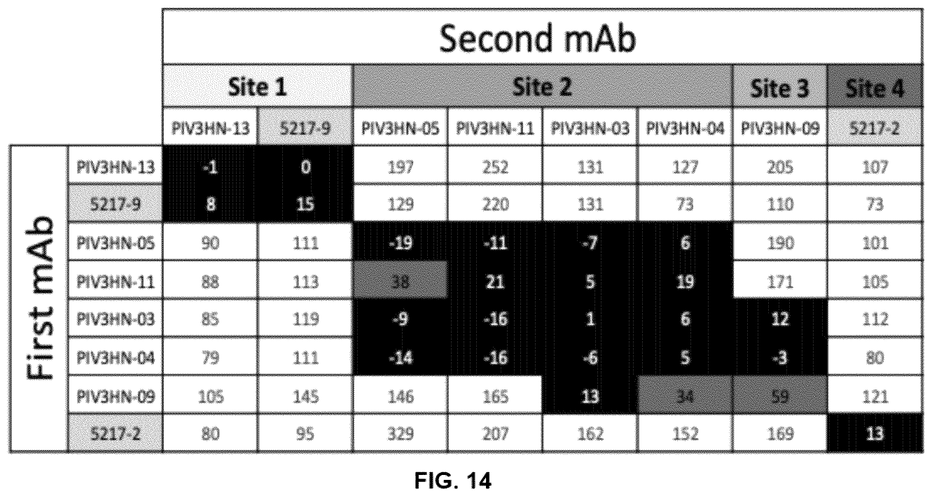

To further investigate the antigenic binding profiles of the newly characterized mAbs alongside those previously identified, epitope mapping was performed. Epitope binding groups were identified using biolayer interferometry (BLI), a technique that measures interactions by analyzing interference patterns. The goal was to determine whether the newly isolated mAbs recognized unique epitopes or shared binding sites with the previously described antibodies. Anti-His biosensors were loaded with histidine-tagged PIV3 HN protein and then underwent a series of sequential mAb association steps. Binding of the first mAb was detected by an increase in the wavelength shift. Upon introduction of the second mAb association, competition was indicated by the absence of an additional shift, suggesting overlapping binding epitopes. In contrast, the presence of an additional shift indicated no competition, meaning the antibodies had distinct epitopes. Previously, three epitope groups termed Site 1, Site 2, and Site 3 were mapped using this method. The addition of the newly generated monoclonal antibodies into the binning assay led to the discovery of a novel binding site designated as Site 4. Notably, mAb 5217-2 demonstrated exclusive binding to this new epitope, as evidenced by its unique binding profile and lack of competition with the Site 1-3 antibodies. On the other hand, mAb 5217-9 was found to compete with antibodies classified within Site 1 (PIV3HN-13), indicating its binding to this previously characterized epitope of Site 1 (FIG. 14).

### Novel Site 4 mAb Induces Complement Deposition

In addition to recognizing and neutralizing pathogens through their Fab regions, antibodies also play a crucial role in activating innate immune defenses. Through their Fc region, antibodies interact with complement proteins, initiating a cascade that leads to the formation of the membrane attack complex (MAC) and ultimately cell lysis of the target cell. To assess this function, the newly developed mAbs were tested for their ability to activate complement proteins. Biotinylated PIV3 HN protein was conjugated to fluorescent Neutravidin beads and incubated with individual mAbs. Guinea pig complement C3b was then added to the immune complexes, enabling antibody-mediated complement deposition. Deposition was detected using a FITC-conjugated anti-C3 antibody and then analyzed via flow cytometry. Increased FITC fluorescence indicated higher levels of complement recruitment. Mab 5217-2 demonstrated elevated Fc effector responses when compared to the positive control PIV03.

### Site 3 mAb PIV3HN-09 Binds Opposite of Site 1 mAb PIV3HN-13

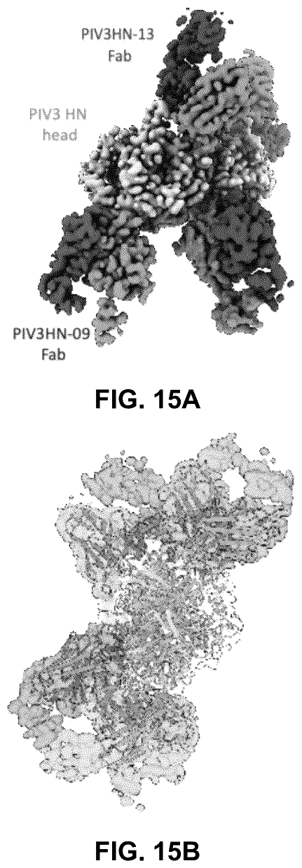

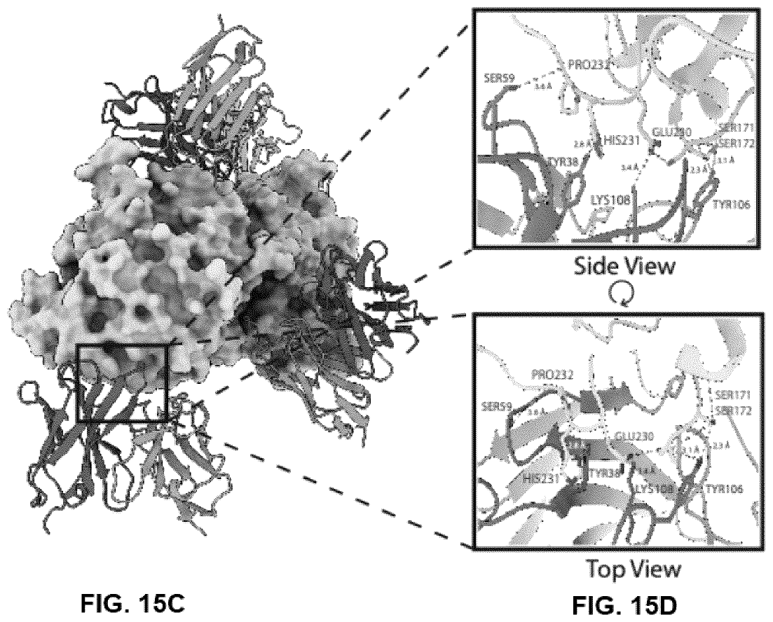

While the structures of monoclonal antibodies targeting Sites 1 and 2 have been previously elucidated, the Site 3 directed mAb, PIV3HN-09, was identified as a potent neutralizer capable of inhibiting F-HN protein fusion. To better understand its binding epitope, it was sought to determine the structure of PIV3HN-09 in complex with PIV3 HN protein using cryo-electron microscopy (cryo-EM). To do this, PIV3HN-09 mAb was cleaved into Fabs via papain digestion and complexed with the HN protein. Complex formation was confirmed by size exclusion chromatography (SEC), after which the sample was prepared for structural analysis using single-particle cryo-EM. The particle movies were processed using CryoSPARC, ultimately allowing refinement of a PIV3 HN dimer in complex with the PIV3HN-09 Fab and the known epitope of the PIV3HN-13 Fab. The final electron density map was resolved at a resolution of 2.95 Å obtained from 198,000 particles and was fit onto a predicted model from AlphaFold3 (FIGS. 15A-15B). Manual model building was performed using Coot, in which amino acid side chains and backbone conformations were adjusted to fit the cryo-EM density map. After model adjustment, refinement and validation were carried out in Phenix, which included evaluation of stereochemistry and Ramachandran outliers.

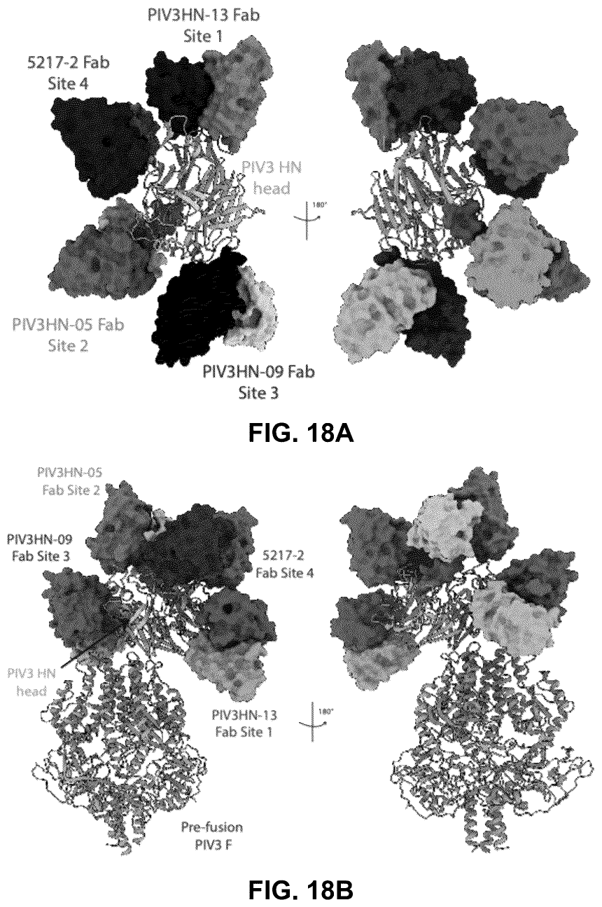

Structural analysis reveals that mAb PIV3HN-09 binds at the opposite end of the Site 1 mAb, PIV3HN-13, near the HN dimer interface (FIG. 18A). Closer examination of the binding interface shows that the epitope is primarily recognized through interactions mediated by the heavy chain of the antibody. All three complementarity-determining region (CDR) loops of the heavy chain contribute to binding by engaging multiple PIV3 HN surface residues. Specifically, the HCDR1 residue Tyr38 forms a hydrogen bond with His231 of the HN protein at a distance of 2.8 Å. Ser59 at the HCDR2 interacts with Pro232 (3.6 Å) of the PIV3 HN protein via a hydrogen bond. Tyr106 on the HCDR3 forms separate hydrogen bonds with Ser171 and Ser 172 (2.3 Å and 3.1 Å, respectively). A final HCDR3 residue Lys108 forms a bond with Glu230 on HN (3.4 Å) (FIG. 15D).

Notably, the light chain of PIV3HN-09 does not appear to contribute to antigen binding, as no significant contacts are observed between light chain residues and the HN protein. To gain further insight into the potential mechanism of neutralization, the structures of the HN-specific monoclonal antibodies were fit onto the PIV3 F-HN complex map (EMD-27550), which captures the conformation of the F and HN proteins in the prefusion state (FIG. 18B). This structural alignment revealed that Fab PIV3HN-09 binds adjacent to the F-HN interaction site, providing structural support for previous findings that PIV3HN-09 inhibits fusion between the F and HN glycoproteins (a likely mechanism underlying its neutralizing activity).

### Site 4 mAb 5217-2 Binds Opposite of the F-HN Binding Interface

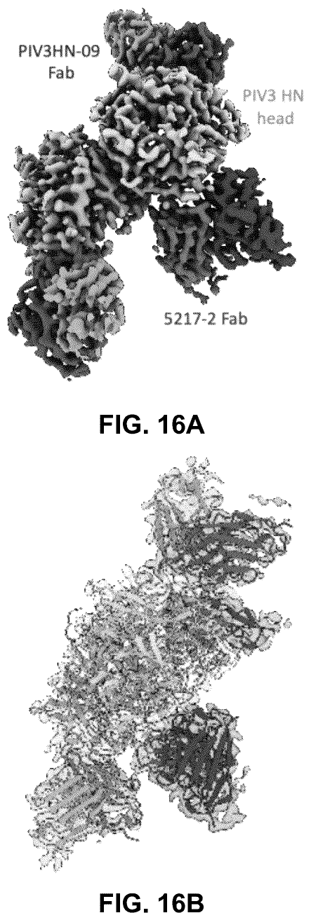

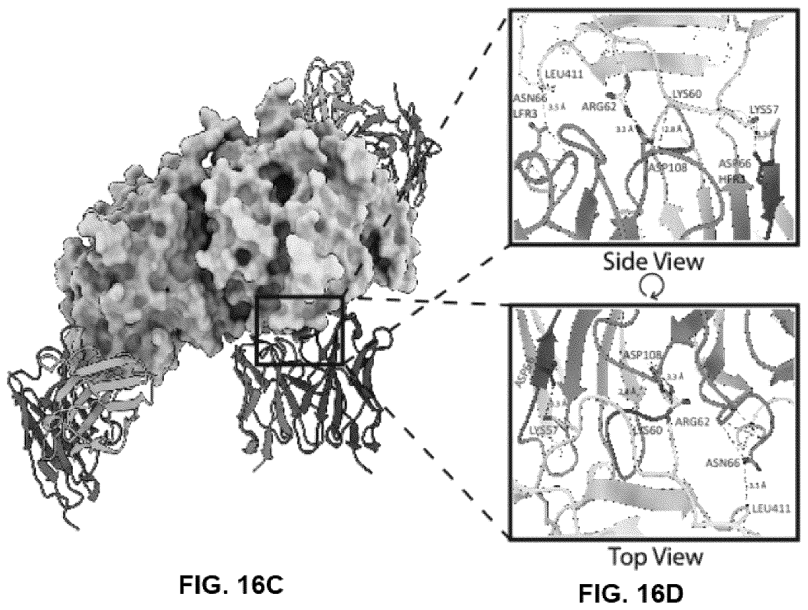

To complete the structural characterization of the four known antigenic sites on the PIV3 HN surface, cryo-electron microscopy was used to resolve the Site 4 epitope interactions of mAb 5217-2. Using the same workflow as previously described, mAb 5217-2 was cleaved to Fabs via papain digestion, complexed with PIV3 HN and PIV3HN-09 Fab, and verified by size-exclusion chromatography. Cryo-EM grids were prepared, and movies were collected for further processing. Following particle picking and heterogeneous refinement, a 3.29 Å resolution electron density map of a PIV3 HN dimer in complex with four Fabs was obtained: two from PIV3HN-09 and two from 5217-2 (FIG. 16A). An initial structural model was generated using Alpha-Fold3 and fit into the electron density map. The model was then manually refined to fit the map in Coot and further optimized in Phenix (FIG. 16B).

Structural analysis revealed that the 5217-2 Fab binds to the apex of the HN head domain, positioned distal to the F-HN binding interface (FIGS. 18A-18B). The interaction is predominantly driven by two heavy chain contacts, with an additional contribution from a single light chain residue. Unlike the PIV3HN-09 epitope, binding of the 5217-2 Fab involves a single CDR loop, with HCDR3 residue Asp108 forming two hydrogen bonds with Arg62 (3.3 Å) and Lys60 (2.8 Å). In addition to the HCDR3 interaction, framework-mediated contacts were observed: HFR3 residue Asp66 forms a hydrogen bond with Lys57 (3.3 Å), and LFR3 residue Asn66 forms a hydrogen bond with Leu 411 (3.5 Å) (FIG. 16D).

Comparison of the Site 4 mAb's binding position relative to the F-HN interface reveals that it is situated adjacent to the Site 2 mAbs (FIG. 18B). While Site 2 mAbs directly occupy the sialic acid binding pocket and block receptor engagement, structural alignment reveals that the Site 4 mAb binds just outside this pocket, adjacent to the receptor binding site. Despite not directly overlapping the sialic acid pocket, the Site 4 mAb 5217-2 exhibits potent neutralizing activity, suggesting that it may interfere with the receptor binding site.

### Updated Map and Model of Site 2 mAb PIV3HN-05

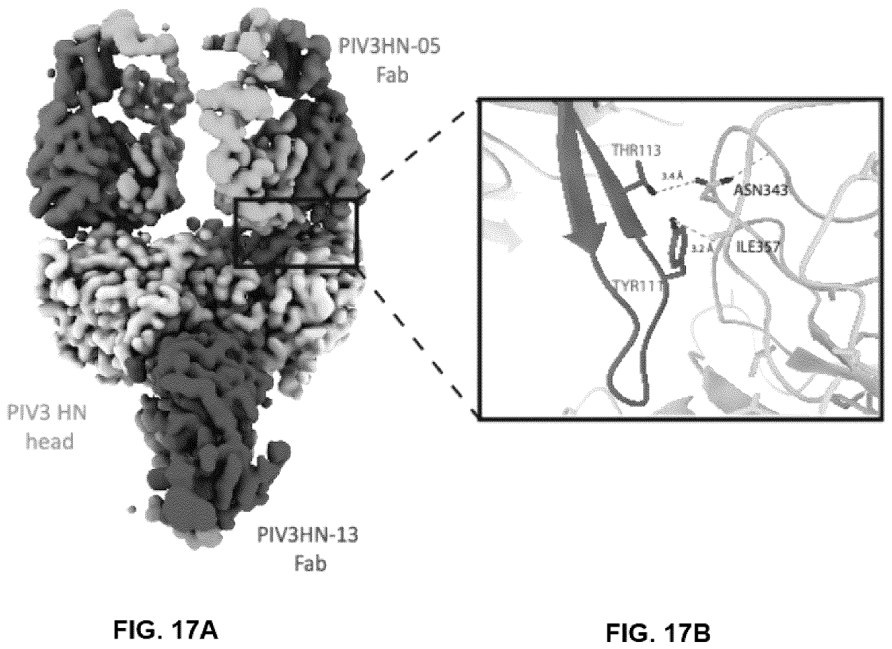

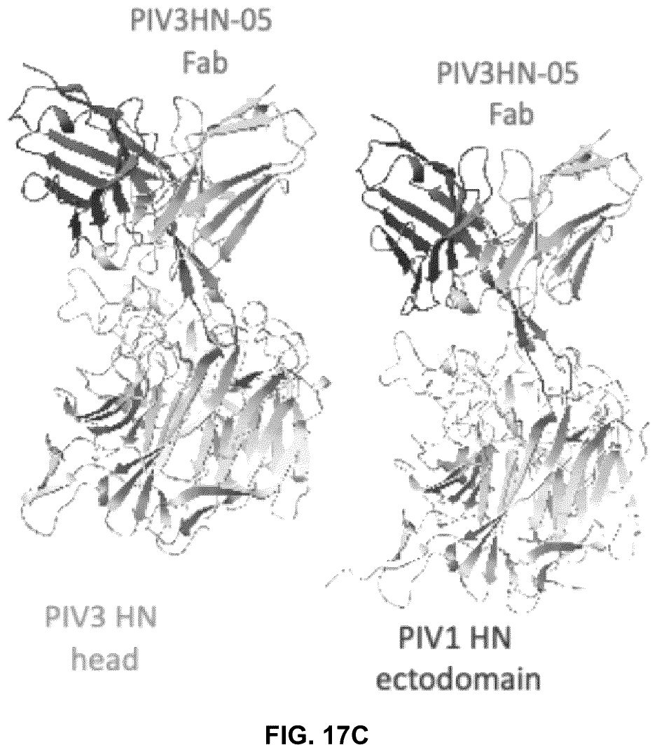

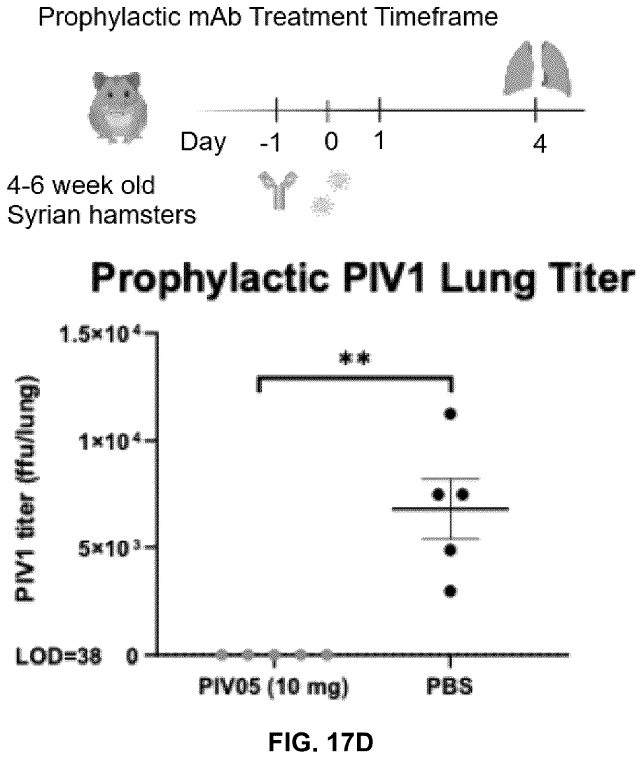

The potent neutralizing activity of Site 2 mAbs is attributed to their targeting of the sialic acid binding site on the HN protein, where they likely interfere with receptor binding and/or prevent viral release by blocking HN's interaction with sialic acid. Notably, the Site 2 mAb, PIV3HN-05 has demonstrated both prophylactic and therapeutic efficacy against PIV3 and exhibited cross neutralization against parainfluenza virus 1, with an IC₅₀ of 180 ng/mL. To better understand the molecular basis for this potent neutralizer, the structural characterization of the PIV3HN-05 epitope was revisited. The initial cryo-EM map revealed disordered regions within the variable region, limiting resolution and key Fab-HN interactions. To address this, a new, higher resolution cryo-EM map of PIV 3HN in complex with PIV3HN-05 Fab and PIV3HN-13 Fab was generated to gain a clearer understanding of the mode of binding (FIG. 17A).

The structure revealed that the interaction between PIV3HN-05 Fab and PIV3 HN is mediated exclusively by the heavy-chain complementarity-determining region 3 (HCDR3) loop. This loop fits deeply within the binding pocket of the HN sialic acid binding site, limiting the HN's ability to bind to the host cell sialic acid-containing receptors. This interaction is formed by two hydrogen bonds between the Fab and HN active site: Thr113 with Asn343 (3.4 Å) and Tyr111 with Ile357 (3.2 Å) (FIG. 17B). These contacts suggest a mechanism in which the Fab physically blocks the sialic acid binding site, therefore inhibiting viral attachment and entry into the host cell. Building on this observation, the HCDR3 loop was overlaid onto PIV1 HN to assess the mechanism behind the cross-reactivity for PIV1 (FIG. 17C). The HCDR3 loop appears to insert into the sialic acid binding pocket of PIV1 HN, suggesting a similar mechanism of neutralization against both PIV1 and PIV3.

To further evaluate the protective efficacy of the PIV3HN-05 mAb against PIV1, an in vivo prophylactic challenge study was conducted. For this study, Syrian golden hamsters were used as the animal model, as they support PIV1 replication in both the lung and nasopharynx. Hamsters (n=10) received an intraperitoneal injection of PIV3HN-05 mab at a dose of 10 mg/kg or PBS as a control, administered 24 hours prior to the intranasal PIV1 challenge. Four days after the challenge, lung samples were harvested and homogenized for viral titration via plaque assay (FIG. 17D, top). Compared to the PBS treated group, which exhibited an average of 6.8×10³ ffu per lung, the PIV3HN-05 mAb treated group showed complete inhibition of viral replication, with no detectable plaques, indicating effective reduction of PIV1 in the lungs (FIG. 17D, bottom). These findings indicate that PIV3HN-05 mAb provides protection not only against PIV3 but also against PIV1, likely due to its ability to block HN binding to host cell receptors through a conserved mechanism of neutralization.

### Discussion

With no approved vaccines or antiviral therapies currently available for human parainfluenza viruses (HPIVs), there is a critical need for the development of effective therapeutic strategies. Neutralizing monoclonal antibodies (mAbs) have shown clinical success against several respiratory viruses, including RSV and influenza, demonstrating their potential to mitigate disease severity in vulnerable populations. However, to date, no monoclonal antibodies have been approved for the treatment or prevention of HPIV infections, highlighting a significant gap in available interventions against this virus. In this study, two mAbs were introduced that target the PIV3 HN antigen and characterized the binding avidity, viral neutralization ability, and structural properties of these mAbs. With this, epitopes on the PIV3 HN protein could be mapped, and the potential of this protein as a viable target for vaccines or monoclonal antibody development was demonstrated.

The addition of newly isolated mAbs in this study led to the identification of a previously uncharacterized epitope on the PIV3 HN protein, designated Site 4. Assessment of the mAbs revealed that both Site 1 and Site 4-targeting antibodies, 5217-2 and 5217-9, bound with high avidity to the HN protein. However, consistent with the previously characterized Site 1 antibodies, 5217-9 exhibited weak neutralizing abilities. In contrast, the Site 4 mAb showed potent neutralizing activity relative to the positive control and was capable of recruiting complement proteins via its Fc-mediated effector functions.

In addition to the efficacy studies, the structures of monoclonal antibodies targeting Site 3 and Site 4 on the PIV3 HN protein were determined. Structural analysis revealed that the Site 3-specific mAb, PIV3HN-09, binds in close proximity to the interface between the HN and F proteins. The binding is primarily mediated by the heavy chain, which binds residues on the HN surface adjacent to the F-interaction interface. The proximity of PIV3HN-09 to the HN-F interface, combined with its ability to inhibit viral fusion, suggests that this mAb may interfere with the HN-mediated activation of the F protein. This supports a possible mechanism in which the antibody prevents fusion by disrupting the HN-F interaction that is required for viral entry. The structure of mAb 5217-2 revealed that Site 4 is located at the apex of the HN head domain. This region is adjacent to the epitope targeted by PIV3HN-05, a potent and cross-neutralizing Site 2 mAb. Notably, structural analysis of the PIV3HN-05 interaction reveals that the HCDR3 loop occupies the sialic acid active site of the HN protein, potentially blocking the HN's ability to bind to sialic acid on the host cell. Given the close proximity of the Site 4 epitope to this receptor-binding pocket along with the promising functional activities of the 5217-2 mAb, this antibody was further pursued for evaluation as a prophylactic treatment in an animal model.

First, the prophylactic efficacy of the Site 4 mAb using Syrian golden hamsters challenged with PIV3 was assessed. Despite its promising structural features and in vitro activity, this antibody did not significantly reduce viral load, indicating limited protection in vivo. In contrast, PIV3HN-05 was also evaluated in vivo against PIV1 as it was only shown to be cross-neutralizing in cellular models. Remarkably, animals that received PIV3HN-05 prophylactically exhibited complete protection, with no detectable viral replication in the lungs. These findings highlight a clear difference in the protective efficacy between antibodies targeting different antigenic sites on the HN protein. Collectively, these results, alongside the previously published findings, indicate that only antibodies targeting Site 2 have demonstrated protection against PIV3 and even PIV1.

In summary, this study offers comprehensive new insights into the antigenic landscape of the PIV3 HN protein, highlighting the distinct roles of various epitopes in mediating antibody protection. By integrating epitope binning, plaque reduction neutralization assays, structural characterizations, and in vivo efficacy studies, it was concluded that Site 2 is still the most promising target for further investigation. These findings emphasize the robust protective capacity of Site 2-targeting antibodies, particularly PIV3HN-05 which binds a critical and conserved region on the PIV3 HN protein. To further understand the basis for its cross-protective activity, future studies should investigate the structural interactions between PIV3HN-05 and the HN protein of PIV1. Additionally, further studies should be done to evaluate the in vivo efficacy and structural interactions of other Site 2 mAbs, such as PIV3HN-11, which was shown to inhibit fusion of PIV3 to host cells. Together, these efforts will help guide the development of broadly protective monoclonal antibodies targeting the HN protein of PIV3, as well as those from other parainfluenza viruses.

### Methods

### Cell Lines

293F cells (ThermoFisher, Cat: R79007) were cultured in Freestyle 293 media (ThermoFisher, Cat: 12338026). ExpiCHO-S cells (ThermoFisher, Cat: A29127) were cultured in ExpiCHO Expression Medium (ThermoFisher, Cat: A2910001). LLC-MK2 cells (ATCC, CCL-7) were cultured in Opti-MEM I+GlutaMAX (ThermoFisher, Cat: 51985034) supplemented with 2% fetal bovine serum.

### Enzyme-Linked Immunosorbent Assay (ELISA)

To measure binding to the PIV3 HN protein, 384-well plates were coated with 2 μg/ml of antigen overnight at 4° C. The plates were blocked for 1 h with 2% non-fat dry milk supplemented with 2% goat serum. Plates were then washed three times with water and primary mAbs were applied to wells for 1 h at 37° C. Following another three times wash with water, a secondary antibody (goat anti-human IgG Fc-AP, Southern BioTech) at a dilution of 1:4000 in 1% blocking solution was added. After incubating for an hour, the plates were washed five times with PBS-T and substrate solution (1 mg/ml PNPP disodium salt hexahydrate, ThermoFisher) was added to each well. The plates were incubated in the dark for 1 h at room temp before reading the optical density at 405 nm on a Biotek plate reader.

### Complement Deposition Cytotoxicity (CDC) Assay

Biotinylated PIV3 HN antigen was coupled with FluoSpheres Neutravidin beads (Invitrogen, Cat: F8775) at a 1:1 ratio of antigen (μg): beads (μL) for 2 h at 37° C. The beads were washed with 5% PBS-BSA and resuspended 1:100 in 0.1% PBS-BSA. The mAbs were diluted to 2.5 μg/mL in a 96 U-bottom plate and incubated for 2 h with 10 μL of antigen-specific beads. The bead-mAb complexes were then washed with 0.1% PBS-BSA. Guinea pig complement (MP Biomedicals, Cat: 64283) was diluted 1:50 in R-10 buffer (RPMI-1640+10% FBS). The diluted complement was then incubated with the bead-mAb complex for 15 mins and then washed twice with PBS. Fluorescein-conjugated goat anti-guinea pig complement c3 (MP Biomedicals, Cat: 55385) was diluted 1:100 in PBS and incubated for 15 min in the dark. Complexes were washed twice with PBS and resuspended in a final volume of 150 μL PBS. Samples were read on the Cytek Aurora.

Flow Cytometry with PIV3-Infected Cells

Biotinylated mAbs were incubated with streptavidin-conjugated PE (BioLegend, Cat: 405204) on ice for 1 h and stored at 4° C. for later use. LLC-MK2 cells were cultured in a T225 flask at 37° C., 5% CO₂ to 80-90% confluency prior to infection. Cells were washed with PBS twice and infected with PIV3 at an MOI of 0.01. The virus was incubated with the cells for 1 h at 37° C., 5% CO₂ with gentle rocking every 10 mins. Virus culture media (OptiMEM+5 μg/mL trypsin-EDTA+100 μg/mL CaCl₂+1% 100× anti-anti) was added to the flask and incubated for 48 hours. After incubation, the cells were digested using Versene (Gibco, Cat:15040-066) at 37° C., 5% CO₂ for 45 mins. Cells were washed twice with FACS buffer and incubated in FACS for 30 mins on ice. Aliquots of 1 million cells were incubated with their respective PE-conjugated mAbs at 2.5 μg/mL for 30 mins on ice in the dark. Cells were again washed twice with FACS buffer and fixed for 15 mins with 4% paraformaldehyde. Finally, the cells were resuspended in 1 mL of FACS buffer. Samples were read on the Cytek Aurora.

### PIV3 mAb Plaque Reduction Neutralization Assay

LLC-MK2 cells were seeded in 24-well flat bottom plates and incubated at 37° C., 5% CO₂ for 2 days to reach confluency. Following the incubation period, 5217-2 and 5217-9 mAbs were serially diluted and incubated at a 1:1 ratio with PIV3 for 1 hour at room temperature. The media was removed from the 24-well plates and 50 μL/well of the virus/mAb mixture was added on top of the cells and rocked at 37° C., 5% CO₂ for 1 h. Cells were then overlaid with of 0.75% methylcellulose dissolved in Opti-MEM with 5 μg/mL trypsin-EDTA and 100 μg/mL CaCl₂ and incubated for 4 days. After incubation, the cells were fixed with 10% neural buffered formalin for 45 mins and washed once with water. The plaques were immuno-stained by first blocking the plates for 1 h with block comprised of 2% non-fat milk and 2% goat serum in PBS-T. The plates were washed 2 times with water and the primary mAb, PIA174, was then added at a dilution of 1 μg/mL in block and incubated for 1 h at room temp. After washing 2 times with water, HRP-conjugated goat anti-human IgG (Southern BioTech, Cat: 2048-05) diluted 1:2000 in block was added for an hour. The plates were washed 3 times with water and TrueBlue peroxidase substrate was added to each well and rocked for no longer than 10 mins and washed once with water. The plates were allowed to dry overnight and were manually counted using a stereoscope. IC₅₀ values were calculated using GraphPad Prism10.

### Epitope Binning

Biolayer interferometry on a Gator Prime instrument was performed for epitope binning. Anti-His (HIS) Probes (GatorBio, PN:160009) were equilibrated in kinetics buffer. A baseline measurement was recorded by dipping the biosensors into wells containing the kinetics buffer for 60 s. Next, biosensors were loaded with PIV3 HN at a concentration of 100 μg/mL for 150 s followed by another baseline measurement. The loaded biosensors were then dipped into wells containing either mAb PIV01, PIV03, PIV04, PIV05, PIV09, PIV11, PIV13, 5217-2, or 5217-9 at a concentration of 100 μg/ml for 300 s. The biosensors were then dipped into another set of the same mAbs listed previously for 300 s to test for a second association. Each antibody was tested for competition against itself and all other antibodies, with assays performed in both directions by using each antibody in both the first and second association steps. Relevant mAb nucleotide and amino acid sequences are provided in Table 8, where nucleotide sequences have SEQ ID NOs. 114-133 and amino acid sequences have SEQ ID NOs. 134-153, respectively:

TABLE 8
HC and LC Sequences for PIV1F, PIV3F, and PIV3HN mAbs

| Category | Description | Nucleotide Sequence | Amino Acid Sequence |
|---|---|---|---|
| PIV1F | FS1-1557HC | ATGAAACATCTCTGGTTCTTTCTCCTGTTGGTAGCAGCGCCCC | QVRLVESGGGVVQPGGSLRLS |
| mAbs | | GCTGGGTGTTGAGCCAGGTCCGGCTGGTGGAATCAGGGGGC | CETSGFSLSSFGMHWVRQAP |
| | | GGGGTGGTACAGCCTGGGGGTAGCCTCCGACTCTCCTGTGA | GKGLEWVAFIRDDGRNTNYA |
| | | AACTTCCGGCTTTTCACTTTCCTCCTTTGGGATGCATTGGGTCC | DSVKGRFTISRDISKNTLYLEMK |
| | | GGCAGGCCCCCGGAAAAGGGCTCGAGTGGGTAGCCTTTATT | SLRVEDTATYYCAKKGASNHYF |
| | | AGAGACGATGGGCGTAACACTAACTACGCCGACTCCGTCAAG | YIDVWGRGTTVTVSS |
| | | GGCCGCTTTACAATCAGCCGCGATATATCTAAGAACACTCTCT | |
| | | ATCTCGAGATGAAATCACTGCGCGTTGAGGACACGGCCACAT | |
| | | ATTACTGCGCCAAGAAGGGCGCCAGTAATCACTACTTTTACAT | |
| | | CGACGTTTGGGGTCGGGGCACTACTGTTACCGTTAGCTCT | |
| | FS1-1557KC | ATGAAACATCTGTGGTTTTTCCTTCTCCTCGTGGCCGCTCCCC | EAVLTQSPGTLSLSPGERATLSC |
| | | GCTGGGTGCTTTCAGAGGCCGTGCTCACACAGAGCCCCGGA | RASQSVSSSFLAWYQHKPGQA |
| | | ACCTTGTCCCTGTCCCCAGGCGAGAGAGCAACTCTTTCCTGTA | PRLLIYGASNRATGIPDRFSGSG |
| | | GAGCCAGTCAGTCAGTGTCTAGCTCATTCCTGGCGTGGTACC | SGADFTLTITSLEPEDFAVYYCH |
| | | AGCACAAACCTGGGCAGGCTCCTCGCCTTTTGATATATGGCG | QYGSALKWTFGQGTKVEIK |
| | | CTTCAAACAGGGCTACCGGAATCCCGGACCGGTTTTCAGGAT | |
| | | CCGGGTCTGGTGCAGATTTTACACTGACCATAACAAGTTTGG | |
| | | AGCCAGAGGACTTTGCTGTGTACTATTGTCATCAATATGGAA | |
| | | GTGCCTTGAAGTGGACTTTCGGTCAGGGCACAAAAGTAGAG | |
| | | ATTAAA | |
| | FS1-1832HC | ATGAAGCACCTGTGGTTTTTCCTTCTTCTTGTGGCGGCACCTC | QVTLKESGPALVKPTQTLTLTC |
| | | GTTGGGTCCTCAGTCAGGTGACACTGAAGGAGAGCGGGCCT | TFSGFSLSTSGMRVSWIRQPP |
| | | GCCTTGGTTAAGCCAACGCAGACACTCACCTTGACATGCACG | GKALEWLARIDWDDDKYYSTS |
| | | TTCTCAGGCTTCAGCCTCTCCACCTCTGGGATGAGGGTGTCCT | LKTRLTISKDTSNNQVVLTMTN |
| | | GGATCCGACAACCCCCCGGGAAAGCCCTCGAATGGCTGGCC | MDPADTATYYCARSYSNSWYS |
| | | AGAATCGATTGGGACGACGATAAGTATTACTCCACTTCCCTG | WFDPWGQGTLVTVSS |
| | | AAGACGAGGCTGACCATTAGTAAGGACACCTCCAATAACCAG | |
| | | GTTGTGTTAACAATGACCAATATGGATCCTGCAGATACAGCG | |
| | | ACCTATTACTGCGCTCGGTCTTATAGCAACTCCTGGTATTCAT | |
| | | GGTTTGATCCATGGGGCCAGGGGACACTGGTGACAGTGAGT | |
| | | TCC | |
| | FS1-1832KC | ATGAAGCACCTCTGGTTTTTCTTGCTCCTTGTCGCTGCCCCTCG | DIQMTQSPSSLSASAGDRVTIT |
| | | GTGGGTGCTGAGTGATATCCAAATGACCCAGAGCCCCAGTTC | CRASQSISSFLNWYQQKQGKA |
| | | CCTTTCAGCCAGCGCCGGGGATAGAGTGACCATTACTTGCCG | PNLLIYAASSLQSGVPSRFSGSG |
| | | GGCCAGTCAGTCTATCAGCAGCTTCCTCAATTGGTACCAGCA | SGTDFTLTISSLQPEDFATYYCQ |
| | | AAAACAAGGCAAGGCACCGAACCTTCTGATCTATGCAGCTTC | QSYSIPYTFGQGTKLEIK |
| | | CAGCCTGCAGTCAGGCGTGCCCAGCCGGTTCTCCGGGAGTG | |
| | | GGAGCGGTACTGATTTTACCTTAACTATCAGTTCCCTTCAGCC | |
| | | CGAGGATTTCGCAACGTATTATTGTCAGCAGTCTTACTCCATC | |
| | | CCCTATACCTTTGGTCAGGGAACCAAGCTGGAGATTAAA | |
| PIV3F | FS1-38HC | ATGAAGCACCTGTGGTTCTTTCTCCTCCTGGTGGCCGCCCCGC | EVQLVQSGAEVKKPGESLKISC |
| mAbs | | GGTGGGTGCTGTCCGAGGTGCAATTGGTACAAAGTGGCGCT | KGSGYIFTTYWIGWVRQMPG |
| | | GAGGTTAAGAAACCAGGAGAGTCCCTGAAAATATCTTGCAA | KGLEWMGIIYPGDSDIRYSPSF |
| | | GGGCTCCGGATACATTTTTACCACCTATTGGATCGGCTGGGT | EGQVTISADKSISTAYLQWPSL |
| | | GCGCCAAATGCCAGGTAAAGGTCTGGAGTGGATGGGCATAA | KASDTAMYYCAILDGMGVWG |
| | | TTTACCCGGGGGACTCCGACATCCGGTACTCCCCTTCTTTCGA | QGTTVTVSS |
| | | GGGACAAGTGACAATTAGCGCCGATAAATCAATTAGCACCGC | |
| | | CTATCTTCAGTGGCCCAGTCTCAAGGCTTCCGATACTGCCATG | |
| | | TATTATTGTGCGATCCTGGACGGAATGGGCGTGTGGGGACA | |
| | | AGGAACTACAGTGACCGTCTCAAGT | |
| | FS1-38LC | ATGAAGCATCTCTGGTTCTTTCTGCTTCTCGTCGCAGCTCCTA | QAVLTQPSSLSASPGASASLTC |
| | | GGTGGGTACTGAGTCAGGCGGTGTTGACGCAGCCCTCCTCTT | TLRSGINVAIYMIHWYQQKPG |
| | | TATCTGCGAGCCCAGGCGCTAGCGCTTCACTGACCTGTACTTT | SPPQYLLRYKSDSDKQQGSGV |
| | | GCGCTCTGGGATAAACGTGGCCATATATATGATTCATTGGTA | PSRFSGSKDASANAGILLISGLQ |
| | | TCAGCAGAAGCCAGGCTCCCCCCCCCAGTATCTGTTAAGGTA | SEDEADYYCLIWHSNAWVFG |
| | | TAAAAGCGATTCTGATAAACAACAGGGATCAGGCGTTCCGTC | GGTKLTVL |
| | | ACGGTTTAGCGGTAGTAAAGACGCTAGTGCCAACGCTGGGA | |
| | | TTCTTCTGATCTCCGGGCTCCAGAGCGAGGACGAGGCTGATT | |
| | | ACTATTGTCTTATCTGGCATAGTAACGCCTGGGTTTTTGGTGG | |
| | | GGGCACGAAACTCACAGTACTG | |
| | FS1-503HC | ATGAAGCACCTGTGGTTTTTTCTTTTGCTGGTGGCCGCTCCTA | EVQLVESGGGLVQPGGSLRLS |
| | | GATGGGTTCTGTCCGAAGTGCAGCTGGTTGAGAGTGGTGGT | CAASGFTFSTYWMTWVRQAP |
| | | GGATTGGTTCAACCGGGTGGAAGTCTCCGTCTGAGTTGCGCC | GKGLEWVANIKEDGSEKHYVD |
| | | GCGTCTGGATTCACTTTCTCAACCTATTGGATGACGTGGGTG | SVKGRFTISRDNAKNSLYLQM |
| | | AGACAGGCACCTGGAAAAGGCCTGGAATGGGTCGCCAATAT | NSLRDEDTAVYYCARGMARD |
| | | TAAAGAGGACGGTTCTGAAAAGCATTATGTCGACTCCGTGAA | PWGQGTLVAVSS |
| | | GGGCCGCTTCACTATCTCACGCGATAACGCCAAAAACTCACTT | |
| | | TATCTCCAGATGAATTCTCTGCGGGACGAGGACACAGCAGTA | |
| | | TATTACTGTGCCAGAGGTATGGCCAGAGACCCATGGGGCCA | |
| | | GGGTACATTGGTCGCCGTGAGCAGC | |
| | FS1-503KC | ATGAAGCATTTGTGGTTCTTTCTTCTTCTCGTGGCTGCGCCTC | DIQMTQSPSTLSASVGDKVTIT |
| | | GCTGGGTCCTCTCTGACATCCAGATGACTCAAAGCCCCTCCAC | CRASQTIGSWLAWYQQKPGK |
| | | TCTTAGCGCTAGCGTCGGCGACAAAGTCACGATCACATGCAG | APNLLIYMASTLESGVPSRFSG |
| | | AGCATCTCAGACCATTGGCTCCTGGTTAGCGTGGTATCAACA | SGSGTEFTLTISSLQPDDFATYY |
| | | AAAGCCCGGCAAGGCCCCCAACCTGCTGATTTATATGGCTAG | CHQYDSYPYTFGRGTKLEIK |
| | | CACACTGGAGTCCGGTGTCCCATCCAGATTTTCCGGATCTGG | |
| | | AAGCGGCACAGAGTTCACTCTTACCATCAGCTCCCTTCAACCA | |
| | | GATGACTTTGCTACCTACTATTGCCATCAATACGACTCCTATC | |
| | | CCTATACATTTGGACGGGGGACAAAATTGGAAATAAAA | |
| | FS1-600HC | ATGAAACACCTGTGGTTCTTCCTTCTCTTAGTAGCGGCCCCTC | EVQLVQSGAEVKKPGESLKISC |
| | | GGTGGGTCCTGTCAGAGGTGCAATTGGTGCAGAGTGGGGCG | KGSGYSFTSNWIAWVRQMPG |
| | | GAGGTCAAAAAGCCCGGGGAATCTCTCAAGATTAGCTGCAA | KGLEWMGVIYPGDSDTRYRPS |
| | | GGGATCCGGGTATAGCTTTACTTCCAACTGGATCGCATGGGT | FQGQVTISADKSISTAYLQWSS |
| | | TAGACAGATGCCTGGCAAAGGGCTGGAGTGGATGGGCGTGA | LKASDTAMYYCARRGSRGFDY |
| | | TTTATCCAGGGGACTCTGACACAAGGTACAGGCCCAGTTTCC | WGQGTLVTVTVSS |
| | | AGGGACAGGTGACTATCAGCGCCGACAAGTCCATCAGCACT | |
| | | GCCTATCTGCAGTGGAGTAGTCTGAAGGCCAGTGACACCGCC | |
| | | ATGTATTATTGCGCACGCAGAGGGAGTAGAGGCTTCGACTAC | |
| | | TGGGGCCAGGGGACCCTGGTGACAGTAACCGTGTCCTCT | |
| | FS1-600LC | ATGAAACACCTGTGGTTTTTCCTGCTTCTCGTTGCCGCTCCAC | YELSQPPSVSVSPGQTASITCSA |
| | | GGTGGGTTCTTAGCTACGAGCTGTCTCAGCCGCCATCCGTAT | DKLGNENVCWYQQKPGQSPV |
| | | CTGTTAGCCCAGGTCAGACCGCCTCTATTACCTGTAGCGCCG | VVIYQNNKRPSGIPERFSGSKS |
| | | ACAAGCTCGGCAACGAGAACGTATGTTGGTATCAGCAAAAAC | GNTATLTISGTQAMDEADYYC |
| | | CTGGCCAGTCCCCCGTGGTTGTAATTTATCAAAATAACAAGA | QSWDSSTAEVFGGGTKLTVL |
| | | GACCCAGCGGAATCCCCGAGCGTTTCTCCGGCTCCAAGAGCG | |
| | | GAAACACTGCAACCTTAACGATCAGTGGGACCCAAGCCATGG | |
| | | ATGAAGCAGATTACTACTGCCAGTCCTGGGACTCTAGCACCG | |
| | | CCGAGGTCTTCGGAGGAGGAACGAAGCTCACAGTCCTC | |
| | FS1-687 HC | ATGAGAGCCTGGATCTTTTTCCTGCTGTGCCTGGCTGGGCGC | EVQLVQSGAEVKKPGESLKISC |
| | | GCCCTGGCCGAGGTCCAACTTGTTCAGAGCGGTGCTGAGGT | KGSGYSFTSNWIGWVRQMPG |
| | | GAAAAAACCCGGCGAATCACTGAAGATATCCTGCAAGGGGA | KGLEWMGIIYPGDSDTRYNPP |
| | | GTGGTTACTCCTTCACTAGTAACTGGATCGGGTGGGTAAGGC | FQGQVTMSADKSISAAFLQWS |
| | | AGATGCCCGGCAAGGGGCTGGAATGGATGGGGATCATTTAT | SLKASDTAMYYCARQSSRGFD |
| | | CCCGGCGATTCTGATACACGATATAACCCGCCCTTTCAGGGC | YWGQGTLVIVSS |
| | | CAGGTCACTATGAGTGCAGATAAAAGCATTTCAGCAGCCTTT | |
| | | CTGCAGTGGTCTAGCCTCAAAGCTTCCGATACTGCTATGTACT | |
| | | ATTGTGCGAGGCAATCCAGCAGGGGTTTCGATTACTGGGGG | |
| | | CAGGGCACTCTTGTTATCGTCAGCTCC | |
| | FS1-687 LC | ATGAGAGCCTGGATCTTTTTCCTGCTGTGCCTGGCTGGGCGC | QSVLTQPPSVSGAPGQRVTISC |
| | | GCCCTGGCCCAGTCTGTGCTCACTCAGCCCCCTAGCGTCAGT | TGSSSNIGAGYNVHWYQQLP |
| | | GGCGCCCCGGGCCAGCGTGTTACCATTAGCTGTACCGGGTCA | GTAPKLLIYGNNNRPSGVPDRF |
| | | TCCTCCAATATAGGGGGGGGCTATAACGTGCATTGGTATCAG | SGSKSGTSASLAITGLQAEDEA |
| | | CAGCTCCCGGGAACAGCACCTAAACTGCTTATATATGGTAAT | DYYCQSYDTSLSGSGVFGGGT |
| | | AATAATAGGCCATCCGGCGTGCCCGATAGGTTTAGCGGATCA | KLTVL |
| | | AAGTCAGGGACTAGCGCAAGCCTGGCAATTACTGGACTTCAA | |
| | | GCCGAGGATGAAGCCGATTACTACTGCCAGTCCTACGATACA | |
| | | TCCCTGTCCGGGTCCGGCGTTTTTGGTGGGGGCACAAAGCTC | |
| | | ACCGTCCTG | |
| | FS1-2719 HC | ATGAGAGCCTGGATCTTTTTCCTGCTGTGCCTGGCTGGGCGC | QVHLVESGGGVVQPGRSLRLS |
| | | GCCCTGGCCCAGGTTCACCTCGTGGAGTCTGGAGGCGGGGT | CVASGFTFSTYGMHWVRQAP |
| | | GGTCCAGCCCGGCCGGTCTCTCCGGCTGAGCTGCGTTGCCTC | GKGLEWVAIISDDGSNESYADS |
| | | TGGGTTTACATTCTCCACATACGGCATGCACTGGGTGAGGCA | AKGRFTISRDNSRNTVHLQMN |
| | | GGCTCCGGGGAAAGGCTTGGAGTGGGTCGCTATCATTAGCG | SLKTEDTAVYYCGNGYRNYWN |
| | | ATGATGGATCCAATGAGTCCTACGCTGATTCTGCCAAGGGTA | DIRGGPLYKWGQGTMVTVSS |
| | | GGTTTACTATATCTAGGGACAACTCCAGGAATACCGTCCATCT | |
| | | GCAGATGAATTCTCTGAAGACTGAGGACACGGCCGTGTATTA | |
| | | TTGTGGCAACGGATATAGAAACTATTGGAATGACATTCGAGG | |
| | | GGGCCCCCTGTATAAGTGGGGTCAGGGCACCATGGTGACTG | |
| | | TCAGTTCC | |
| | FS1-2719 LC | ATGAGAGCCTGGATCTTTTTCCTGCTGTGCCTGGCTGGGCGC | SYVLTQPPSVSVAPGQTATITC |
| | | GCCCTGGCCAGCTACGTCTTAACCCAGCCTCCTTCTGTAAGTG | GGNRVGSKSVHWYQQKPGQ |
| | | TCGCCCCCGGACAGACCGCTACAATCACGTGCGGGGGAAAT | APVLVVYDDSDRPSGIPERFSG |
| | | AGGGTCGGTTCCAAGTCAGTCCACTGGTATCAGCAGAAGCCT | SNFGNTATLTITGVEAGDEADY |
| | | GGACAGGCACCGGTCCTTGTGGTGTACGACGACAGCGACCG | YCQVWDNNGVHPHVIFGGGA |
| | | GCCCTCCGGAATCCCCGAGAGGTTCTCTGGTTCTAACTTTGG | KLTVL |
| | | GAATACAGCTACACTGACTATCACTGGCGTCGAGGCCGGTGA | |
| | | CGAAGCCGATTACTACTGCCAAGTATGGGATAACAATGGTGT | |
| | | GCATCCCCACGTAATTTTTGGCGGTGGCGCGAAACTGACCGT | |
| | | TCTG | |
| | FS1-3484 HC | ATGAGAGCCTGGATCTTTTTCCTGCTGTGCCTGGCTGGGCGC | QVQLQQWGAGLLTPSETLSLT |
| | | GCCCTGGCCCAGGTGCAGCTCCAACAATGGGGCGCAGGACT | CAVFGGSFSDYYWSWIRQSPG |
| | | TCTCACACCCAGCGAAACGCTCTCACTGACCTGCGCCGTGTTT | KGLEWIGEINHSGDKTYNPSLQ |
| | | GGCGGTTCATTCAGCGATTACTATTGGTCCTGGATACGTCAAT | SRIAISVDTSKKQFSLKLNSVTA |
| | | CACCAGGCAAAGGACTGGAGTGGATCGGCGAAATCAATCAC | ADTAVYFCARQGRVTTFGVLIE |
| | | TCTGGGGATAAAACATACAATCCATCACTGCAATCTCGCATCG | GFFDLWGRGTLVTVSS |
| | | CGATTAGCGTAGACACTTCAAAGAAGCAGTTTAGTCTGAAAC | |
| | | TTAATTCCGTCACAGCTGCCGACACAGCGGTCTATTTCTGTGC | |
| | | CCGCCAAGGCCGGGTTACAACCTTTGGCGTGTTGATCGAGGG | |
| | | GTTCTTTGATCTGTGGGGCAGAGGGACGCTTGTAACAGTGAG | |
| | | TAGC | |
| | FS1-3484 KC | ATGAGAGCCTGGATCTTTTTCCTGCTGTGCCTGGCTGGGCGC | DIQMTQSPSSLSASVGDRVTII |
| | | GCCCTGGCCGATATACAGATGACCCAGTCTCCCTCTAGTTTGA | CRASQNIDTYLNWYQQKPGK |
| | | GTGCTTCTGTGGGAGACCGCGTAACGATTATATGCCGGGCTT | APKFLIYAASTLQNGVPSRFSG |
| | | CCCAGAACATCGACACGTACCTGAATTGGTATCAGCAAAAGC | SGSGTDFTLTINSLQPEDFATYF |
| | | CTGGGAAGGCACCTAAGTTCCTGATCTACGCTGCCAGTACTC | CQQSHSTPLTFGGGTRVEIK |
| | | TCCAAAATGGAGTTCCGTCCAGGTTCTCTGGATCCGGCAGCG | |
| | | GCACTGATTTTACTTTAACTATTAACAGTCTGCAGCCTGAGGA | |
| | | TTTCGCTACATATTTCTGTCAACAGAGTCACTCCACACCCCTG | |
| | | ACGTTTGGGGGCGGAACCCGCGTGGAAATTAAG | |
| PIV3HN | 5217-2 HC | GAGGTGCACCTCCTGGAGTCTGGGGGAGGCCTGGTCCAGCC | EVHLLESGGGLVQPGGSRRISC |
| mAbs | | TGGGGGGTCCCGCAGAATCTCCTGTGCAGCCTCTGGATTCTC | AASGFSFGSYAMSWVRQAPG |
| | | TTTTGGCAGCTATGCCATGAGCTGGGTCCGCCAGGCTCCAGG | KGLEWVSTITSSGESTDYADSV |
| | | GAAGGGGCTGGAGTGGGTCTCGACTATAACTAGTAGTGGTG | KGRFIISRDNAKNTLDLLMNSL |
| | | AAAGCACAGACTACGCAGACTCCGTGAAGGGCCGGTTCATCA | RAGDTAVYYCAKGGAVVLSA |
| | | TCTCCAGAGACAACGCCAAGAACACGCTGGATCTCCTAATGA | MDVWGQGTTVTVSS |
| | | ACAGCCTGAGAGCCGGGGACACGGCCGTTTACTACTGTGCG | |
| | | AAGGGGGGGGCAGTAGTACTGAGCGCTATGGACGTCTGGG | |
| | | GCCAAGGGACTACGGTCACCGTCTCCTCA | |
| | 5217-2 LC | GACATCCAGTTGACCCAGTCTCCATCTTCCCTGTCTGCATCTG | DIQLTQSPSSLSASVGDRVTITC |
| | | TAGGAGACAGGGTCACCATCACTTGCCAGGCGAGTCACGAC | QASHDISNYLNWYQQKPGKA |
| | | ATTAGTAACTATCTAAATTGGTATCAGCAGAAACCAGGGAAA | PKLLIYDASNLETGVPPRFSGG |
| | | GCCCCTAAGCTCCTGATCTACGATGCATCCAATTTGGAAACAG | GSGTHFSLTISSLQPEDIATYYC |
| | | GGGTCCCACCAAGGTTCAGTGGAGGTGGATCTGGGACACAT | QQDDSLPLTFGPGTKVEIK |
| | | TTTAGTCTCACCATCAGCAGCCTGCAGCCTGAAGACATTGCAA | |
| | | CATATTACTGTCAACAGGATGATAGTCTCCCATTAACTTTCGG | |
| | | CCCTGGGACCAAGGTGGAAATCAAA | |
| | 5217-9 HC | GAGGTGCAGCTGTTGGAGTCGGGGGGAGGCTTGGTACAGCC | EVQLLESGGGLVQPGGSLRLSC |
| | | TGGGGGGTCCCTGAGACTCTCCTGTGCAGCCTCTGGATTCAC | AASGFTFSSHAMSWVRQAPG |
| | | CTTTAGCAGCCATGCCATGAGCTGGGTCCGCCAGGCTCCAGG | KGLEWVSTIGSSGISTYYTDSVK |
| | | GAAGGGGCTGGAGTGGGTCTCAACTATTGGTAGTAGTGGCA | GRFTISRDNSKNTLFLQLNSLR |
| | | TTAGTACATACTACACAGACTCCGTGAAGGGCCGCTTCACCAT | AEDTAVYYCEVGGDQIASSYW |
| | | CTCCAGAGACAATTCCAAGAACACGCTGTTTCTTCAACTGAAC | GQGTLVTVSS |
| | | AGCCTGAGAGCCGAGGACACGGCCGTCTATTACTGTGAAGT | |
| | | GGGGGGGGATCAAATCGCGTCTTCCTACTGGGGCCAGGGAA | |
| | | CCCTGGTCACCGTCTCCTCA | |
| | 5217-9 LC | GACATCCAGATGACCCAGTCTCCTTCCACCCTGTCTGCATCTG | DIQMTQSPSTLSASVGDRVTIT |
| | | TTGGGGATAGAGTCACCATCACTTGCCGGGCCAGTGAGAATA | CRASENINSWLAWYQQKPGK |
| | | TTAATAGTTGGTTGGCCTGGTATCAGCAGAAACCAGGGAAAG | APKLLIYKASSLQSGVPSRFSGS |
| | | CCCCTAAACTCCTGATCTACAAGGCGTCTAGTTTACAAAGTGG | RSGTEFTLTISSLQPDDFATYYC |
| | | GGTCCCATCAAGGTTCAGCGGCAGTAGATCTGGGACAGAATT | QQYNTYFSYTFGQGTKLEIK |
| | | CACTCTCACCATCAGTAGCCTGCAGCCTGATGATTTTGCAACT | |
| | | TATTACTGCCAACAGTATAATACTTATTTCTCGTACACTTTTGG | |
| | | CCAGGGGACCAAGTTGGAGATCAAA | |

### PBMC Isolation

Leukofilters were emptied into sterile tubes and flushed twice with phosphate-buffered saline (PBS). The collected blood was carefully layered on top of pre-warmed Lymphocyte Separation Media (Corning, Cat: 25-072-CV) and then centrifuged at 2500 RPM for 30 mins using minimal brake and acceleration (set at 1) to not disturb the layers. The plasma was removed, and the buffy coat was carefully extracted and added to Dulbecco's Modified Eagle Medium (DMEM). The PBMCs were washed twice with DMEM (300×g for 15 mins) and then resuspended at a concentration of 100 million cells/mL. PBMCs were aliquoted into cryovials and DMSO was added to minimize cell damage during freezing. Cryovials were placed in a cryogenic freezing container and stored at −80° C. overnight. For long time preservation, samples were transferred to liquid nitrogen storage.

### 5217-2 and 5217-9 mAb Discovery

Peripheral blood mononuclear cells (PBMCs) from blood of human donors were blocked with Trustain FcX (Biolegend, Cat: 422302) for 30 minutes, washed once in PBS, and subsequently stained according to the following protocol: 5 uL CD19-APC (Biolegend, Cat: 302212), 1.25 uL IgM-FITC (Southern Biotech, Cat: 2020-02), 1.25 uL IgD-FITC (Southern Biotech, Cat: 2030-02), 2.5 uL GhostRed710 (Cyteck Biosciences, Cat: 13-0871-T100), and 5 μL of PIV3 HN conjugated to PE (Biolegend, Cat: 405204). The cells were stained for 30 minutes, washed twice in PBS, and immediately sorted on Moflo Astrios EQ (Beckman Coulter) 10× sequencing (Pleasanton, CA) to obtain paired heavy and light chain variable sequences. The heavy (in a pTwist CMV hlgG1 vector), kappa (in a pTwist CMV hlgK vector), and lambda (in a pTwist CMV hlgL2 vector) variable sequences were cloned into their respective plasmids from Twist Biosciences (San Francisco, CA).

Plasmid Transformation into E. coli Competent Cells

Plasmids from 10× sequences were ordered from Twist BioScience and transformed into E. Coli DH5a competent cells. The cells were incubated with plasmid DNA (1 μL) and heat shocked before incubating in LB broth medium for 1 h and plated on ampicillin agar plates overnight. Isolated colonies were then added to a 5 mL culture of LB broth with ampicillin (1:1000) and then expanded to a 200 mL culture 8 hours later to shake overnight. The DNA plasmids were extracted using the E.Z.N.A Plasmid DNA Maxi Kit (Omega Bio-Tek, Cat: D6922-04) according to the manufacturer's instructions. The resulting plasmid DNA was sterile filtered for later use in transfections.

Plasmid Transfection into Freestyle 293-F Cells and ExpiCHO Cells

Both Freestyle 293-F cells and ExpiCHO cells were used to transfect the PIV mAbs. For Freestyle 293-F transfections, heavy and light chain plasmids were diluted in Opti-MEM medium at a final concentration of 1 μg/ml relative to the culture volume. The transfection reagent PEI MAX (Kyfora Bio, Cat: 24765) was added at a ratio based on the culture volume and incubated with the plasmid mixture for 30 m before adding to the cells. Transfections using ExpiCHO-S cells (ThermoFisher, Cat: A29127) were performed according to the ExpiFectamine CHO transfection kit (ThermoFisher, Cat: A29129) based on the Max Titer Protocol.

Purification of mAbs

Monoclonal antibodies were purified seven days post-transfection in Freestyle 293-F cells or fourteen days post-transfection in ExpiCHO cells. Cultures were centrifuged at 10,000 RPM for 15 mins then the supernatant was passed through a 0.45 μm filter. A HiTrap Protein G column (Cytiva, Cat: 17040503) was used to purify the supernatant according to the manufacturer's instructions. The eluted mAbs were spun in 30 kDa MWCO concentrators (Millipore, Cat: UFC903096) and buffered exchanged with PBS. The concentration was measured on a NanoDrop spectrophotometer using the IgG setting.

### Expression and Purification of Fabs

Fab fragments PIV01, PIV09, PIV13, and 5217-2 were generated by incubating the full length mAbs with immobilized papain overnight at 37° C., following the Pierce Fab Preparation Kit (ThermoFisher, Cat: 44985). The Fc portion was removed using HiTrap MabSelect columns (Cytiva, Cat: 28408253) according to the manufacturer's instructions.

### Production of Antigen+Fab Complexes and Cryo-EM Grid Preparation

Fab fragments (0.7 mg/mL) were combined with PIV3HN (0.35 mg/mL) at a twofold molar excess and incubated overnight at 4° C. The complex was purified on a Superdex200 10/300 SEC column with 50 mM Tris-HCl pH 7.5 and 150 mM NaCl. The purified complex peak was confirmed using SDS-PAGE gel stained with Coomassie blue and concentrated to 1 mg/mL for further use. A FEI Vitrobot MK4 was used to prep sample onto Quantifoil R 1.2/1.3 300 copper mesh grids (Ted Pella, Cat: 658-300-CU). The grids were glow-discharged before adding 5 μL of sample and fast incubated before being blotted and plunged into liquid ethane.

### Cryo-EM Data Collection and Processing

After screening the prepared grids, 4000 movies (PIV09 complex) and 6300 movies (5217-2 complex) were collected on Titan-Krios 300 KV microscope equipped with Gatan-K3 camera. Movies were then imported into CryoSPARC package where patch motion correction and patch CTF estimation were performed. Blob picker was used to pick particles, which then were purified using multiple 2D classification cycles. Selected particles then were used to make multiple ab-initio 3D reconstruction which were used for further 3D classification (heterogenous refinement in CryoSPARC) to obtain the final particle set. Final 3D reconstruction was performed using the non-uniform refinement job to obtain a 2.95 Å (PIV09 complex) and a 3.29 Å (5217-2 complex) resolution map. The sharpened map from this refinement was used for model building and model refinement, and the sharpened map from DeepEMhancer was used for visualizations and figures.

### Model Building and Validation

PIV3 HN dimer model and Fab aa sequences were all imported into Alphafold3, output models then were used to fit into the CryoEM map using ChimeraX. After fitting and fixing the backbone and sidechains with precision into the map using Coot, PHENIX was used to do a 3D real-space refinement and validation.

### Prophylactic/Therapeutic Treatment and Viral Challenge of Syrian Hamsters

Four- to six-week-old female Golden Syrian hamsters (Charles River Laboratories #049) were individually housed at Florida State University's animal facility. One day before the viral challenge, groups were prophylactically treated with their respective mAb (10 mg/kg) through intraperitoneal (IP) injection. After 24 h, hamsters were anesthetized with isoflurane for 2 min before intranasally (IN) infecting with PIV3 (106 PFU/mL). Hamsters were humanely euthanized using pentobarbital injection 4-days post-infection. Lungs were collected and washed with PBS and added to 2 mL of cold Opti-MEM and homogenized for viral titration. If not used immediately, lung samples were stored at −80° C. before homogenization.

### Syrian Hamster Lungs Viral Titration

After homogenization, the lung homogenate was serially diluted in cold Opti-MEM media. The diluted homogenate was added to LLC-MK2 cell monolayer in a 24-well tissue culture plate (200 μL/well) and rocked for 1 h at 37° C., 5% CO₂. The following steps of the titration assay were performed as described earlier.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Abramson J, et al. Accurate structure prediction of biomolecular interactions with AlphaFold 3. Nature 2024 630:8016 [Internet]. 2024 May 8 [cited 2024 Oct. 16]; 630(8016):493-500.
2. Afonine P V, et al. Real-space refinement in PHENIX for cryo-EM and crystallography. Acta Crystallogr D Struct Biol [Internet]. 2018 Jun. 1 [cited 2024 Oct. 16]; 74(Pt 6):531.
3. Ahani B, et al. Molecular and phenotypic characteristics of RSV infections in infants during two nirsevimab randomized clinical trials. Nature Communications 2023 14:1 [Internet]. 2023 Jul. 19 [cited 2024 Mar. 8]; 14(1):1-10.
4. Banerjee A, et al. Structural basis for ultrapotent antibody-mediated neutralization of human metapneumovirus. Proc Natl Acad Sci USA [Internet]. 2022 Jun. 21 [cited 2023 Jun. 15]; 119(25):e2203326119.
5. Bar-Peled Y, et al. A Potent Neutralizing Site Ill-Specific Human Antibody Neutralizes Human Metapneumovirus In Vivo. J Virol [Internet]. 2019 October [cited 2022 Nov. 27]; 93(19):342-61.
6. Boonyaratanakornkit J, et al. Protective antibodies against human parainfluenza virus type 3 infection. MAbs. 2021; 13(1).
7. Branche, A. R., et al. (2016). Parainfluenza Virus Infection. Seminars in Respiratory and Critical Care Medicine, 37(4), 538-554.
8. Branche A R, et al. Respiratory Viral Infections: Parainfluenza Virus Infection. Semin Respir Crit Care Med [Internet]. 2016 Aug. 1 [cited 2023 Mar. 14]; 37(4):538.
9. Caben M, et al. Cross-protective antibodies against common endemic respiratory viruses. Nature Communications 2023 14:1 [Internet]. 2023 Feb. 13 [cited 2023 Nov. 24]; 14(1):1-15.
10. Cagigi A, et al. Vaccine Generation of Protective Ebola Antibodies and Identification of Conserved B-Cell Signatures. J Infect Dis [Internet]. 2018 Nov. 22 [cited 2024 Mar. 8]; 218(suppl_5):S528-36.
11. Caidi H, et al. RSV growth and quantification by microtitration and qRT-PCR assays. Methods in Molecular Biology [Internet]. 2016 [cited 2024 Feb. 6]; 1442:13-32.
12. Casañal A, et al. Current developments in Coot for macromolecular model building of Electron Cryo-microscopy and Crystallographic Data. Protein Sci [Internet]. 2020 Apr. 1 [cited 2024 Oct. 16]; 29(4):1069.
13. Chang A, et al. Paramyxovirus Fusion and Entry: Multiple Paths to a Common End. Viruses 2012, Vol 4, Pages 613-636 [Internet]. 2012 Apr. 19 [cited 2023 Dec. 1]; 4(4):613-36.
14. Cooper N R, et al. The Role of Antibody and Complement in the Control of Viral Infections. Journal of Investigative Dermatology. 1984 Jul. 1; 83(1):S121-7.
15. Cortez K J, et al. Outbreak of human parainfluenza virus 3 infections in a hematopoietic stem cell transplant population. J Infect Dis [Internet]. 2001 Nov. 1 [cited 2023 Mar. 14]; 184(9):1093-7.
16. Counihan M E, et al. Human parainfluenza virus-associated hospitalizations among children less than five years of age in the United States. Pediatr Infect Dis J [Internet]. 2001 [cited 2023 Mar. 14]; 20(7):646-53.
17. Cruz-Teran, C., et al. (2021). Challenges and opportunities for antiviral monoclonal antibodies as COVID-19 therapy. Advanced Drug Delivery Reviews, 169, 100-117.
18. DeGroote N P, et al. Human parainfluenza virus circulation, United States, 2011-2019. Journal of Clinical Virology [Internet]. 2020 Mar. 1 [cited 2023 Nov. 16]; 124:104261.
19. Dunkelberger J R, et al. Complement and its role in innate and adaptive immune responses. Cell Research 2010 20:1 [Internet]. 2009 Dec. 15 [cited 2023 Nov. 16]; 20(1):34-50.
20. Ejemel M, et al. A cocktail of human monoclonal antibodies broadly neutralizes North American rabies virus variants as a promising candidate for rabies post-exposure prophylaxis. Scientific Reports 2022 12:1 [Internet]. 2022 Jun. 7 [cited 2024 Mar. 8]; 12(1):1-11.
21. Elboukari H, et al. Parainfluenza Virus. 2023 Jul. 17. In: StatPearls [Internet]. Treasure Island (FL): StatPearls Publishing; 2025 January-. PMID: 32809554.
22. Feng Z, et al. A multicentre study on the incidence of respiratory viruses in children with community-acquired pneumonia requiring hospitalization in the setting of the zero-COVID policy in China. Arch Virol [Internet]. 2023 Feb. 1 [cited 2023 Mar. 14]; 168(2):1-8.
23. Fischinger S, et al. A high-throughput, bead-based, antigen-specific assay to assess the ability of antibodies to induce complement activation. J Immunol Methods. 2019 Oct. 1; 473:112630.
24. Ganar, K., et al. (2014). Newcastle disease virus: Current status and our understanding. Virus Research, 184, 71-81.
25. Garg R, et al. Vaccination with a human parainfluenza virus type 3 chimeric FHN glycoprotein formulated with a combination adjuvant induces protective immunity. Vaccine. 2017 Dec. 18; 35(51):7139-46.
26. Goldberg B S, et al. Antibody-mediated complement activation in pathology and protection. Immunol Cell Biol [Internet]. 2020 Apr. 1 [cited 2023 Nov. 16]; 98(4):305-17.
27. Greer C E, et al. Long-term Protection in Hamsters against Human Parainfluenza Virus Type 3 Following Mucosal or Combinations of Mucosal and Systemic Immunizations with Chimeric Alphavirus-based Replicon Particles. Scand J Immunol [Internet]. 2007 Dec. 1 [cited 2024 Jan. 30]; 66(6):645-53.
28. Guthmiller J J, et al. An efficient method to generate monoclonal antibodies from human B cells. Methods in Molecular Biology [Internet]. 2019 [cited 2024 Feb. 6]; 1904:109-45.
29. Homaira, N. et al, Effectiveness of Palivizumab in Preventing RSV Hospitalization in High Risk Children: A Real-World Perspective, International Journal of Pediatrics, 2014, 571609, 13 pages, 2014.
30. Huang J, et al. Broadly reactive human monoclonal antibodies targeting the pneumococcal histidine triad protein protect against fatal pneumococcal infection. Infect Immun [Internet]. 2021 May 1 [cited 2024 Jan. 30]; 89(5).
31. Huang K, et al, 2010. Respiratory Syncytial Virus-Neutralizing Monoclonal Antibodies Motavizumab and Palivizumab Inhibit Fusion. J Virol 84: doi: 10.1128/jvi.02699-09
32. Human Parainfluenza National Trends—NREVSS|CDC [Internet]. [cited 2023 Nov. 16].
33. Human Parainfluenza Viruses (HPIV) Seasons|CDC [Internet]. [cited 2023 Nov. 24].
34. Ison, M. G., et al. (2016). Noninfluenza Respiratory Viruses. Infectious Diseases (Vol. 12, pp. 1472-1482).
35. Karron, R. A., et al. (2015). Evaluation of a Live-Attenuated Human Parainfluenza Type 1 Vaccine in Adults and Children. Journal of the Pediatric Infectious Diseases Society, 4(4), e143-e146.
36. Khayyata S H, et al. Parainfluenza Virus. Viruses and the Lung: Infections and Non-Infectious Viral-Linked Lung Disorders [Internet]. 2022 Jul. 18 [cited 2023 Mar. 14]; 9783642406058:87-93.
37. Kim H M, et al. Recent increase in the detection of human parainfluenza virus during the coronavirus disease-2019 pandemic in the Republic of Korea. Virol J [Internet]. 2022 Dec. 1 [cited 2023 Mar. 14]; 19(1):1-12.
38. Lawrence M C, et al. Structure of the Haemagglutinin-neuraminidase from Human Parainfluenza Virus Type Ill. J Mol Biol [Internet]. 2004 Jan. 30 [cited 2024 Mar. 8]; 335(5):1343-57.
39. Marcink T C, et al. Human parainfluenza virus fusion complex glycoproteins imaged in action on authentic viral surfaces. PLoS Pathog [Internet]. 2020 Sep. 1 [cited 2023 Mar. 14]; 16(9):e1008883.
40. Marcink T C, et al. Subnanometer structure of an enveloped virus fusion complex on viral surface reveals new entry mechanisms. Sci Adv [Internet]. 2023 Feb. 10 [cited 2023 Mar. 14]; 9(6).
41. Matsuse H, et al. Naturally Occurring Parainfluenza Virus 3 Infection in Adults Induces Mild Exacerbation of Asthma Associated with Increased Sputum Concentrations of Cysteinyl Leukotrienes. Int Arch Allergy Immunol [Internet]. 2005 Nov. 1 [cited 2023 Nov. 27]; 138(3):267-72.
42. Meng E C, et al. UCSF ChimeraX: Tools for structure building and analysis. Protein Science [Internet]. 2023 Nov. 1 [cited 2024 Oct. 16]; 32(11):e4792.
43. Miller, R. J., et al. The structural basis of protective and nonprotective human monoclonal antibodies targeting the parainfluenza virus type 3 hemagglutinin-neuraminidase. Nat Commun 15, 10825 (2024).
44. Moscona A. Entry of parainfluenza virus into cells as a target for interrupting childhood respiratory disease. J Clin Invest [Internet]. 2005 Jul. 1 [cited 2024 Mar. 8]; 115(7):1688-98.
45. Ono N, et al. Measles Viruses on Throat Swabs from Measles Patients Use Signaling Lymphocytic Activation Molecule (CDw150) but Not CD46 as a Cellular Receptor. J Virol [Internet]. 2001 May [cited 2024 Mar. 8]; 75(9):4399-401.
46. Pace C S, et al. Anti-CD4 monoclonal antibody ibalizumab exhibits breadth and potency against HIV-1, with natural resistance mediated by the loss of a V5 glycan in envelope. J Acquir Immune Defic Syndr (1988) [Internet]. 2013 Jan. 1 [cited 2024 Mar. 8]; 62(1):1-9.
47. Pai, M. C., et al. (2024). Clinical characteristics and risk factors of severe human parainfluenza virus infection in hospitalized children. Journal of Microbiology, Immunology and Infection, 57(4), 573-579.
48. Palermo L M, et al. Human Parainfluenza Virus Infection of the Airway Epithelium: Viral Hemagglutinin-Neuraminidase Regulates Fusion Protein Activation and Modulates Infectivity. J Virol [Internet]. 2009 July [cited 2023 Nov. 27]; 83(13):6900-8.
49. Pecchini R, et al. Parainfluenza virus as a cause of acute respiratory infection in hospitalized children. The Brazilian Journal of Infectious Diseases [Internet]. 2015 Jul. 1 [cited 2023 Mar. 14]; 19(4):358.
50. Porotto M, et al. A Second Receptor Binding Site on Human Parainfluenza Virus Type 3 Hemagglutinin-Neuraminidase Contributes to Activation of the FusionMechanism. J Virol [Internet]. 2007 Apr. [cited 2023 Nov. 16]; 81(7):3216-28.
51. Porotto M, et al. Human Parainfluenza Virus Type 3 HN-Receptor Interaction: Effect of 4-Guanidino-Neu5Ac2en on a Neuraminidase-Deficient Variant. J Virol [Internet]. 2001 Aug. 15 [cited 2023 Nov. 27]; 75(16):7481-8.
52. Porotto, M., et al. (2012). Mechanism of fusion triggering by human parainfluenza virus type Ill: Communication between viral glycoproteins during entry. Journal of Biological Chemistry, 287(1), 778-793.
53. Porotto M, et al. Spring-Loaded Model Revisited: Paramyxovirus Fusion Requires Engagement of a Receptor Binding Protein beyond Initial Triggering of the Fusion Protein. J Virol [Internet]. 2011 Dec. 15 [cited 2023 Dec. 1]; 85(24):12867-80.
54. Porotto M, et al. Triggering of Human Parainfluenza Virus 3 Fusion Protein (F) by the Hemagglutinin-Neuraminidase (HN) Protein: an HN Mutation Diminishes the Rate of F Activation and Fusion. J Virol [Internet]. 2003 Mar. 15 [cited 2023 Dec. 1]; 77(6):3647-54.
55. Punjani A, et al. cryoSPARC: algorithms for rapid unsupervised cryo-EM structure determination. Nature Methods 2017 14:3 [Internet]. 2017 Feb. 6 [cited 2024 Oct. 16]; 14(3):290-6.
56. Schmidt, A. C., et al. (2011). Progress in the development of human parainfluenza virus vaccines. In Expert Review of Respiratory Medicine (Vol. 5, Issue 4, pp. 515-526). Expert Reviews Ltd.
57. Schmidt A C. Progress in Respiratory Virus Vaccine Development. Semin Respir Crit Care Med [Internet]. 2011 [cited 2023 Mar. 14]; 32(4):527.
58. Starkie, D. O., et al. (2016). Generation of Recombinant Monoclonal Antibodies from Immunised Mice and Rabbits via Flow Cytometry and Sorting of Antigen-Specific IgG+ Memory B Cells. PloS one, 11(3), e0152282.
59. Stewart-Jones G B E, et al. Structure-based design of a quadrivalent fusion glycoprotein vaccine for human parainfluenza virus types 1-4. Proc Natl Acad Sci USA [Internet]. 2018 Nov. 27 [cited 2022 Dec. 21]; 115(48):12265-70.
60. Streltsov V A, et al. Catalytic mechanism and novel receptor binding sites of human parainfluenza virus type 3 hemagglutinin-neuraminidase (hPIV3 HN). Antiviral Res. 2015 Nov. 1; 123:216-23.
61. Takimoto T, et al. Role of the Hemagglutinin-Neuraminidase Protein in the Mechanism of Paramyxovirus-Cell Membrane Fusion. J Virol [Internet]. 2002 Dec. 15 [cited 2023 Nov. 16]; 76(24):13028-33.
62. Tappert M M, et al. Quantitative Comparison of Human Parainfluenza Virus Hemagglutinin-Neuraminidase Receptor Binding and Receptor Cleavage. J Virol [Internet]. 2013 Aug. 15 [cited 2023 Nov. 27]; 87(16):8962-70.
63. van Erp, E. A., et al. (2019). Fc-Mediated Antibody Effector Functions During Respiratory Syncytial Virus Infection and Disease. Frontiers in immunology, 10, 548.
64. Wang L, et al. Epidemiology and clinical severity of the serotypes of human parainfluenza virus in children with acute respiratory infection. Virol J [Internet]. 2023 Dec. 1 [cited 2023 Dec. 1]; 20(1):1-7.
65. Weinberg G A, et al. Parainfluenza Viral Disease. Goldman's Cecil Medicine: Twenty Fourth Edition. 2012 Jan. 1; 2:2092-5.
66. Weinberg, G. A., et al. (2009). Parainfluenza Virus Infection of Young Children: Estimates of the Population-Based Burden of Hospitalization. Journal of Pediatrics, 154(5). Doi: 10.1016/j.jpeds.2008.11.034
67. Wu H, et al. Development of Motavizumab, an Ultra-potent Antibody for the Prevention of Respiratory Syncytial Virus Infection in the Upper and Lower Respiratory Tract. J Mol Biol. 2007 May 4; 368(3):652-65.
68. Xie C B, et al. Complement Membrane Attack Complex: New Roles, Mechanisms of Action, and Therapeutic Targets. Am J Pathol. 2020 Jun. 1; 190(6):1138-50.
69. Xu R, et al. Interaction between the hemagglutinin-neuraminidase and fusion glycoproteins of human parainfluenza virus type Ill regulates viral growth in vivo. mBio [Internet]. 2013 Oct. 22 [cited 2023 Dec. 1]; 4(5).
70. Zhang L, et al. Infection of Ciliated Cells by Human Parainfluenza Virus Type 3 in an In Vitro Model of Human Airway Epithelium. J Virol [Internet]. 2005 Jan. 15 [cited 2023 Nov. 27]; 79(2):1113-24.
71. Zhang S, et al. Human Parainfluenza Virus Type 3 Matrix Protein Reduces Viral RNA Synthesis of HPIV3 by Regulating Inclusion Body Formation. Viruses [Internet]. 2018 Mar. 11 [cited 2023 Mar. 14]; 10(3).

What is claimed is:

1. An isolated human antibody that binds to at least one protein from a parainfluenza virus (PIV) selected from PIV1, PIV2, PIV3, PIV4, or any combination thereof.

2. The isolated human antibody of claim 1, wherein the antibody is a monoclonal antibody.

3. The isolated human antibody of claim 1 wherein the at least one protein comprises an envelope protein.

4. The isolated human antibody of claim 3, wherein the envelope protein comprises hemagglutinin-neuraminidase or fusion protein.

5. The isolated human antibody of claim 1, wherein a heavy chain of the isolated human antibody has an amino acid sequence selected from SEQ ID NOs. 62-65, SEQ ID NOs. 66-70, or SEQ ID NO. 134, 136, 138, 140, 142, 144, 146, 148, 150, or 152.

6. The isolated human antibody of claim 1, wherein a light chain of the isolated human antibody has an amino acid sequence selected from SEQ ID NOs. 80-83, SEQ ID NOs. 85-88, or SEQ ID NO. 135, 137, 139, 141, 143, 145, 147, 149, 151, or 153.

7. The isolated human antibody of claim 1, wherein the isolated human antibody has at least one mutation in a heavy chain of the isolated human antibody in an amino acid sequence selected from SEQ ID NOs. 62-65, SEQ ID NOs. 66-70, or SEQ ID NO. 134, 136, 138, 140, 142, 144, 146, 148, 150, or 152, or at least one mutation in a light chain of the isolated human antibody in an amino acid sequence selected from SEQ ID NOs. 80-83, SEQ ID NOs. 85-88, or SEQ ID NO. 135, 137, 139, 141, 143, 145, 147, 149, 151, or 153, or both.

8. The isolated human antibody of claim 7, wherein the at least one mutation increases complement deposition activity, antibody-dependent phagocytosis, antibody-dependent cellular cytotoxicity, antibody half-life, or any combination thereof.

9. The isolated human antibody of claim 1, wherein the isolated human antibody is bispecific or trispecific.

10. A pharmaceutical composition comprising the isolated human antibody claim 1 and a pharmaceutically acceptable carrier.

11. The pharmaceutical composition of claim 10, wherein the pharmaceutical composition is formulated to be administered intranasally, by inhalation, or by injection.

12. A method for passive immunization of a subject against a parainfluenza virus (PIV), the method comprising administering the pharmaceutical composition of claim 10 to the subject.

13. The method of claim 12, wherein the passive immunization is prophylactic, therapeutic, or both.

14. The method of claim 12, where the parainfluenza virus comprises PIV1, PIV2, PIV3, PIV4, or any combination thereof.

15. The method of claim 12, wherein performing the method inhibits viral fusion with a plurality of cells in the subject, induces complement deposition, inhibits hemagglutination of red blood cells in the subject, prevents replication of the PIV in the subject, or any combination thereof.

16. A method for detecting a parainfluenza virus (PIV) in a subject, the method comprising:
(a) obtaining a sample from the subject;
(b) contacting the sample with the isolated human antibody of claim 1; and
(c) detecting a signal from the sample;
wherein presence of the signal indicates that the PIV is present in the subject and wherein absence of the signal indicates that the PIV is not present in the subject.

17. The method of claim 16, wherein the method comprises an ELISA assay.

18. The method of claim 16, wherein the subject is a human.

19. The method of claim 16, wherein the sample comprises a nasal swab or lung fluid.

20. The method of claim 16, wherein the signal comprises a color change, a fluorescence signal, a luminescence signal, a resonance unit change, a wavelength shift change, or any combination thereof.


SEQUENCE LISTING (153 sequences)

SEQ ID NO: 1 - DNA, 24 bp, synthetic construct
  source 1..24: mol_type = other DNA
acaggtgcccactcccaggtgcag

SEQ ID NO: 2 - DNA, 23 bp, synthetic construct
  source 1..23: mol_type = other DNA
aaggtgtccagtgtgargtgcag

SEQ ID NO: 3 - DNA, 27 bp, synthetic construct
  source 1..27: mol_type = other DNA
cccagatgggtcctgtcccaggtgcag

SEQ ID NO: 4 - DNA, 24 bp, synthetic construct
  source 1..24: mol_type = other DNA
caaggagtctgttccgaggtgcag

SEQ ID NO: 5 - DNA, 22 bp, synthetic construct
  source 1..22: mol_type = other DNA
tcttgtccaccttggtgttgct

SEQ ID NO: 6 - DNA, 25 bp, synthetic construct
  source 1..25: mol_type = other DNA
atgaggstcccygctcagctgctgg

SEQ ID NO: 7 - DNA, 28 bp, synthetic construct
  source 1..28: mol_type = other DNA
ctcttcctcctgctactctggctcccag

SEQ ID NO: 8 - DNA, 25 bp, synthetic construct
  source 1..25: mol_type = other DNA
atttctctgttgctctggatctctg

SEQ ID NO: 9 - DNA, 24 bp, synthetic construct
  source 1..24: mol_type = other DNA
gtttctcgtagtctgctttgctca

SEQ ID NO: 10 - DNA, 23 bp, synthetic construct
  source 1..23: mol_type = other DNA
ggtcctgggcccagtctgtgctg

SEQ ID NO: 11 - DNA, 23 bp, synthetic construct
  source 1..23: mol_type = other DNA
ggtcctgggcccagtctgccctg

SEQ ID NO: 12 - DNA, 23 bp, synthetic construct
  source 1..23: mol_type = other DNA
gctctgtgacctcctatgagctg

SEQ ID NO: 13 - DNA, 23 bp, synthetic construct
  source 1..23: mol_type = other DNA
ggtctctctcscagcytgtgctg

SEQ ID NO: 14 - DNA, 23 bp, synthetic construct
  source 1..23: mol_type = other DNA
gttcttgggccaattttatgctg

SEQ ID NO: 15 - DNA, 23 bp, synthetic construct
  source 1..23: mol_type = other DNA
ggtccaattcycaggctgtggtg

SEQ ID NO: 16 - DNA, 23 bp, synthetic construct
  source 1..23: mol_type = other DNA
gagtggattctcagactgtggtg

SEQ ID NO: 17 - DNA, 24 bp, synthetic construct
  source 1..24: mol_type = other DNA
caccagtgtggccttgttggcttg

SEQ ID NO: 18 - DNA, 18 bp, synthetic construct
  source 1..18: mol_type = other DNA
sargtgcagctcgtggag

SEQ ID NO: 19 - DNA, 18 bp, synthetic construct
  source 1..18: mol_type = other DNA
gaggtgcagctgttggag

SEQ ID NO: 20 - DNA, 38 bp, synthetic construct
  source 1..38: mol_type = other DNA
ctgcaaccggtgtacattccgaggtgcagctggtgcag

SEQ ID NO: 21 - DNA, 38 bp, synthetic construct
  source 1..38: mol_type = other DNA
ctgcaaccggtgtacattcccaggtgcagctgcaggag

SEQ ID NO: 22 - DNA, 25 bp, synthetic construct
  source 1..25: mol_type = other DNA
agtagtccttgaccaggcagcccag

SEQ ID NO: 23 - DNA, 24 bp, synthetic construct
  source 1..24: mol_type = other DNA
atgacccagwctccabycwccctg

SEQ ID NO: 24 - DNA, 22 bp, synthetic construct
  source 1..22: mol_type = other DNA
gtgctgtccttgctgtcctgct

SEQ ID NO: 25 - DNA, 38 bp, synthetic construct
  source 1..38: mol_type = other DNA
ctgctaccggttcctgggcccagtctgtgctgackcag

SEQ ID NO: 26 - DNA, 38 bp, synthetic construct
  source 1..38: mol_type = other DNA
ctgctaccggttcctgggcccagtctgccctgactcag

SEQ ID NO: 27 - DNA, 38 bp, synthetic construct
  source 1..38: mol_type = other DNA
ctgctaccggttctgtgacctcctatgagctgacwcag

SEQ ID NO: 28 - DNA, 37 bp, synthetic construct
  source 1..37: mol_type = other DNA
ctgctaccggttctctctcscagcytgtgctgactca

SEQ ID NO: 29 - DNA, 38 bp, synthetic construct
  source 1..38: mol_type = other DNA
ctgctaccggttcttgggccaattttatgctgactcag

SEQ ID NO: 30 - DNA, 38 bp, synthetic construct
  source 1..38: mol_type = other DNA
ctgctaccggttccaattcycagrctgtggtgacycag

SEQ ID NO: 31 - DNA, 28 bp, synthetic construct
  source 1..28: mol_type = other DNA
ctcctcactcgagggygggaacagagtg

SEQ ID NO: 32 - DNA, 57 bp, synthetic construct
  source 1..57: mol_type = other DNA
atcctttttctagtagcaactgcaaccggtgtacattccgaggtgcagctggtgcag

SEQ ID NO: 33 - DNA, 57 bp, synthetic construct
  source 1..57: mol_type = other DNA
atcctttttctagtagcaactgcaaccggtgtacattctgaggtgcagctggtggag

SEQ ID NO: 34 - DNA, 57 bp, synthetic construct
  source 1..57: mol_type = other DNA
atcctttttctagtagcaactgcaaccggtgtacattctgaggtgcagctgttggag

SEQ ID NO: 35 - DNA, 57 bp, synthetic construct
  source 1..57: mol_type = other DNA
atcctttttctagtagcaactgcaaccggtgtacattcccaggtgcagctgcaggag

SEQ ID NO: 36 - DNA, 59 bp, synthetic construct
  source 1..59: mol_type = other DNA
atcctttttctagtagcaactgcaaccggtgtacattcccaggtgcagctacagcagtg

SEQ ID NO: 37 - DNA, 57 bp, synthetic construct
  source 1..57: mol_type = other DNA
atcctttttctagtagcaactgcaaccggtgtacattctgaagtgcagctggtggag

SEQ ID NO: 38 - DNA, 57 bp, synthetic construct
  source 1..57: mol_type = other DNA
atcctttttctagtagcaactgcaaccggtgtacattcccaggtacagctgcagcag

SEQ ID NO: 39 - DNA, 59 bp, synthetic construct
  source 1..59: mol_type = other DNA
atcctttttctagtagcaactgcaaccggtgtacattctgacatccagatgacccagtc

SEQ ID NO: 40 - DNA, 60 bp, synthetic construct
  source 1..60: mol_type = other DNA
atcctttttctagtagcaactgcaaccggtgtacattcagacatccagttgacccagtct

SEQ ID NO: 41 - DNA, 59 bp, synthetic construct
  source 1..59: mol_type = other DNA
atcctttttctagtagcaactgcaaccggtgtacattgtgccatccggatgacccagtc

SEQ ID NO: 42 - DNA, 59 bp, synthetic construct
  source 1..59: mol_type = other DNA
atcctttttctagtagcaactgcaaccggtgtacatggggatattgtgatgacccagac

SEQ ID NO: 43 - DNA, 59 bp, synthetic construct
  source 1..59: mol_type = other DNA
atcctttttctagtagcaactgcaaccggtgtacatggggatattgtgatgactcagtc

SEQ ID NO: 44 - DNA, 59 bp, synthetic construct
  source 1..59: mol_type = other DNA
atcctttttctagtagcaactgcaaccggtgtacattcagaaattgtgttgacacagtc

SEQ ID NO: 45 - DNA, 59 bp, synthetic construct
  source 1..59: mol_type = other DNA
atcctttttctagtagcaactgcaaccggtgtacattcagaaatagtgatgacgcagtc

SEQ ID NO: 46 - DNA, 60 bp, synthetic construct
  source 1..60: mol_type = other DNA
atcctttttctagtagcaactgcaaccggtgtacattcagaaattgtgttgacgcagtct

SEQ ID NO: 47 - DNA, 59 bp, synthetic construct
  source 1..59: mol_type = other DNA
atcctttttctagtagcaactgcaaccggtgtacattcggacatcgtgatgacccagtc

SEQ ID NO: 48 - DNA, 57 bp, synthetic construct
  source 1..57: mol_type = other DNA
atcctttttctagtagcaactgcaaccggttcctgggcccagtctgtgctgackcag

SEQ ID NO: 49 - DNA, 57 bp, synthetic construct
  source 1..57: mol_type = other DNA
atcctttttctagtagcaactgcaaccggttcctgggcccagtctgccctgactcag

SEQ ID NO: 50 - DNA, 57 bp, synthetic construct
  source 1..57: mol_type = other DNA
atcctttttctagtagcaactgcaaccggttctgtgacctcctatgagctgacwcag

SEQ ID NO: 51 - DNA, 56 bp, synthetic construct
  source 1..56: mol_type = other DNA
atcctttttctagtagcaactgcaaccggttctctctcscagcytgtgctgactca

SEQ ID NO: 52 - DNA, 57 bp, synthetic construct
  source 1..57: mol_type = other DNA
atcctttttctagtagcaactgcaaccggttcttgggccaattttatgctgactcag

SEQ ID NO: 53 - DNA, 57 bp, synthetic construct
  source 1..57: mol_type = other DNA
atcctttttctagtagcaactgcaaccggttccaattcycagrctgtggtgacycag

SEQ ID NO: 54 - DNA, 47 bp, synthetic construct
  source 1..47: mol_type = other DNA
ggaagaccgatgggcccttggtcgacgcctgaggagacggtgaccag

SEQ ID NO: 55 - DNA, 46 bp, synthetic construct
  source 1..46: mol_type = other DNA
ggaagaccgatgggcccttggtcgacgctgaggagacggtgaccag

SEQ ID NO: 56 - DNA, 48 bp, synthetic construct
  source 1..48: mol_type = other DNA
ggaagaccgatgggcccttggtcgacgctgaagagacggtgaccattg

SEQ ID NO: 57 - DNA, 47 bp, synthetic construct
  source 1..47: mol_type = other DNA
ggaagaccgatgggcccttggtcgacgctgaggagacggtgaccgtg

SEQ ID NO: 58 - DNA, 45 bp, synthetic construct
  source 1..45: mol_type = other DNA
aagacagatggtgcagccaccgtacgtttgatytccaccttggtc

SEQ ID NO: 59 - DNA, 45 bp, synthetic construct
  source 1..45: mol_type = other DNA
aagacagatggtgcagccaccgtacgtttgatatccactttggtc

SEQ ID NO: 60 - DNA, 45 bp, synthetic construct
  source 1..45: mol_type = other DNA
aagacagatggtgcagccaccgtacgtttaatctccagtcgtgtc

SEQ ID NO: 61 - DNA, 41 bp, synthetic construct
  source 1..41: mol_type = other DNA
tgttggcttgaagctcctcactcgagggygggaacagagtg

SEQ ID NO: 62 - protein, 128 aa, synthetic construct
QVQLQEWGPGVVKPSQTLSLTCTVSGGSISSGFYYWRWIRQPAGKGLEWIGRLYTSGSTNYHPSFKSGVTISVDTSKNQFSLKMRCVTAADTAVYYCARSKGYSNDYLKYXXYGMDVWGQGTTVTVSS

SEQ ID NO: 63 - protein, 120 aa, synthetic construct
EVQLVQSGAEMKKPGASVKVSCKASGYTYTNYYIHWVRQAPGLGLEWMGIVNPTGDGTSYAPKFKGRVTMTGDTSTSTVYMELNNLKSDDTAVYYCARARVNGVFESDYWGPGTLVSVSS

SEQ ID NO: 64 - protein, 130 aa, synthetic construct
EVQLVESGGGVVQPGGSLRLSCVASGLSLSPNWMHWVRQAPGKGLVWVSRISDNGDTTNYAGSVMGRFTISRDNAKNTLYLQMNNLRADDTAVYFCVRESAVISSPSWGLYDTAYHFDPWGQGTLVTVSS

SEQ ID NO: 65 - protein, 123 aa, synthetic construct
QVQLQESGPGLVKPSETLSLTCTVSGGSVSSGSYYWSWIRQPPGKGLEWIGDIFTSGSTSYSPSLKSRVTISLDGSKNHFSLKLRSVSSADAAVYYCARESWEGAYLKYFDYWGQGTLVTVSS

SEQ ID NO: 66 - protein, 133 aa, synthetic construct
EVQLVQSGAEVKEPGASVKVSCKASGYISTDYYIHWVRQAPGQGPEWMAILNPGGGSPSYAQRFQDRVSVTRDTSTETVYMELSRLRPEDTAVYYCVRDQYDFWTSYYTGVEGRHYYYGLDVWGQGTTVTVSS

SEQ ID NO: 67 - protein, 123 aa, synthetic construct; identical to SEQ ID NO: 65

SEQ ID NO: 68 - protein, 123 aa, synthetic construct; identical to SEQ ID NO: 65

SEQ ID NO: 69 - protein, 121 aa, synthetic construct
EVQLLESGGALVQPGGSLRVSCAASGFSFSSYAMSWLRQTPGKGLEWVSAIGGSGHSTYYADSVQGRFTVSRDNSKDTLYLQMNSLRAEDTAVYYCAKFFRSDGVFHFDYWGPGNPGSPSP

SEQ ID NO: 70 - protein, 124 aa, synthetic construct
QVQLQESGPGLVKPSETLSLTCTVSGGSISSYYWSWIRQPAGKGLEWIGRIYSSGSTKYSPSLRGRVTMSLDRSKNQFSLKLTSVSAADTAVYYCARDRFIAVAADTRLGMDVWGQGTTVTVSS

SEQ ID NO: 71 - DNA, 384 bp, synthetic construct
  source 1..384: mol_type = other DNA
caggtgcagctgcaggagtggggcccaggagtggtgaagccttcacagaccctgtccctcacctgcactgtctctggtggttccatcagcagtggtttttactactggagatggatccggcagcccgccgggaagggactggagtggattgggcgtttatataccagtgggagcaccaactaccacccctccttcaagagtggagtcaccatatcagtagacacgtccaagaaccagttttccctgaagatgaggtgtgtgaccgccgcagacacggccgtgtattactgtgcgagatccaaggggtacagtaatgattacctcaagtactantantacggcatggacgtctggggccaagggaccacggtcaccgtctcctcg

SEQ ID NO: 72 - DNA, 361 bp, synthetic construct
  source 1..361: mol_type = other DNA
gaggtgcagctggtgcagtctggggctgagatgaagaagcctggggcctcagtgaaggtttcctgcaaggcatctggatacacctacaccaattattacatacactgggtgcgacaggcccctggactcgggcttgagtggatgggaatagtcaaccctaccggtgatggcacaagctacgcaccgaagttcaagggcagagtcaccatgaccggggacacgtccacgagcacggtctacatggaactgaacaacctgaaatctgacgacacggccgtctattactgtgcgagagcgcgcgtgaatggagtattcgaaagtgactactggggcccgggaaccctggtctccgtctcctcag

SEQ ID NO: 73 - DNA, 391 bp, synthetic construct
  source 1..391: mol_type = other DNA
gaggtgcagctggtggagtccgggggaggcgtagttcagccgggggggtccttgagactctcctgtgttgcctctggactcagcctcagccccaattggatgcactgggtccgccaagctccagggaaggggctggtgtgggtctcacgtatcagtgataatggggatacgacaaactacgcgggctccgtgatgggccgattcaccatctccagagacaacgccaagaacacgctgtatctgcaaatgaacaatctgagagccgacgacacggctgtctatttttgtgtgagagagtcggcggtgattagcagcccgtcctggggcctctacgacaccgcataccatttcgacccctggggccagggaaccctggtcaccgtctcctcag

SEQ ID NO: 74 - DNA, 370 bp, synthetic construct
  source 1..370: mol_type = other DNA
caggtgcagctgcaggagtcgggcccaggactggtgaagccttcggagaccctgtccctcacctgcactgtctctggtggctccgtcagcagtggtagttactactggagctggatccggcagcccccagggaagggactggagtggattggggacatcttcaccagtgggagtaccagctacagcccctccctcaagagtcgagtcaccatatcactagacgggtccaagaaccacttctcactgaagctgaggtctgtaagctctgcggacgcggccgtgtattactgtgcgagagaaagttgggaaggcgcctatttaaagtactttgactactggggccagggaaccctggtcaccgtctcctcag

SEQ ID NO: 75 - DNA, 370 bp, synthetic construct; identical to SEQ ID NO: 74
  source 1..370: mol_type = other DNA

SEQ ID NO: 76 - DNA, 399 bp, synthetic construct
  source 1..399: mol_type = other DNA
gaggtgcagctggtgcagtctggggctgaggtgaaggagcctggggcctcagtgaaggtttcctgcaaggcttctggatacatctccaccgactactatatacattgggtgcgacaggcccctggacaagggcctgagtggatggcaatcctcaaccctggtggtggtagcccaagttatgcacagaggttccaggacagagtctccgtgaccagggacacgtccacggaaacagtctacatggagctgagtaggctgagacctgaggacacggccgtgtattattgtgtgagagaccagtacgatttttggactagttattataccggtgtagaggggaggcactactactacggtctggacgtctggggccaagggaccacggtcaccgtctcctca

SEQ ID NO: 77 - DNA, 370 bp, synthetic construct; identical to SEQ ID NO: 74
  source 1..370: mol_type = other DNA

SEQ ID NO: 78 - DNA, 364 bp, synthetic construct
  source 1..364: mol_type = other DNA
gaggtgcagctgttggagtctgggggagccttggtgcagccgggggggtccctgagagtctcctgtgcagcctctggattcagcttcagcagctatgccatgagctggctccgccagactccagggaaggggctggagtgggtctccgctattggtggtagtggtcatagcacatactacgcagactccgtgcagggccggttcaccgtctccagagacaattccaaggacacactgtatctgcaaatgaacagcctgagagccgaggacacggccgtttattactgtgcgaaattcttccgtagtgatggtgtcttccactttgactactgggggccagggaaccctgggtcaccgtctcctc

SEQ ID NO: 79 - DNA, 372 bp, synthetic construct
  source 1..372: mol_type = other DNA
caggtgcagctgcaggagtcgggcccaggactggtgaagccttcggagaccctgtccctcacctgcactgtctctggtggctccatcagtagttactactggagctggatccggcagcccgccgggaagggactggagtggattgggcgtatctatagcagtgggagcaccaagtacagcccctccctcaggggtcgagtcaccatgtcactagacaggtcgaagaaccagttctccctgaagctgacctctgtgagcgccgcggacacggccgtgtattattgtgcgagagataggtttatagcagtggctgcggacacacggctcggtatggacgtctggggccaagggaccacggtcaccgtctcctca

SEQ ID NO: 80 - protein, 108 aa, synthetic construct
DIQMXXXPVSLSASVGDRVTITCQASQDISNYLNWYQQKPGKAPKLLIYDASNLETGVPSRFSGSGSGTDFTFTISSLQPEDIATYYCQHFDNVPMYTFGQGTKLEIT

SEQ ID NO: 81 - protein, 107 aa, synthetic construct
DIQMTQSPSFLSASVGDRVTITCRASQGISTYLAWYQQKPGKAPKLLIYGTSTLQSGVPSRFSGTGSGTEFILTISSLQPEDFATYYCQQLDSYPVTFGGGTRVEIK

SEQ ID NO: 82 - protein, 111 aa, synthetic construct
QSVLTQPPSASGTPGQGVTISCSGSNSNIGTNAVDWYQQFPGTAPRLLIFNDNQRPSGVPDRFSGSRSGTSASLAISGLQSEDEAVYYCATWDDSLNGPVVFGGGTKLTVL

SEQ ID NO: 83 - protein, 107 aa, synthetic construct
EIVLTQSPATLSLSPGERATLSCRASQSVSTYLAWYQQKPGQAPRLLMYDASNRATGIPARFSGSGSGTDFTLTISSLESEDFAVYYCQQRSIRPWTFGQGTKVEIK

SEQ ID NO: 84 - protein, 107 aa, synthetic construct; identical to SEQ ID NO: 83

SEQ ID NO: 85 - protein, 110 aa, synthetic construct
EIVLTQSPGTLSLSPGERATLSCRASQSVGSSYLAWYQQKPGQTPRLLIYGASNRATGIPDRFSGSGSGTDFTLTISGLEPEDFAVYYCQHYGRSPLSSTFGQGTKVEIK

SEQ ID NO: 86 - protein, 107 aa, synthetic construct; identical to SEQ ID NO: 83

SEQ ID NO: 87 - protein, 111 aa, synthetic construct
DIVMTQSPLSLPVTPGEPASISCRSSQSLRHSDGNNYLDWYLQKPGQSPQLLIYLGSNRASGVPDRFSGSGSGSDFTLKISRVEAEDVGVYYCMQALQTPTFGQGTKVEIK

SEQ ID NO: 88 - protein, 108 aa, synthetic construct
SYELTQPPSVSVAPGKTARITCGGNNIGSKSVHWYQQKPGQAPVLVIYYDSDRPSGIPERFSGSNSGNTATLTISRVEAGDEADYYCQVWDSSSDHPVFGGGTKLTVL

SEQ ID NO: 89 - DNA, 325 bp, synthetic construct
  source 1..325: mol_type = other DNA
gacatccagatgacnncnnntccagtctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacctattactgtcaacattttgataatgtccctatgtatacttttggccaggggaccaagctggagatcacac

SEQ ID NO: 90 - DNA, 322 bp, synthetic construct
  source 1..322: mol_type = other DNA
gacatccagatgacccagtctccatccttcctgtctgcatctgtaggggacagagtcaccatcacttgccgggccagtcagggcattagtacttatttagcctggtatcagcaaaaaccagggaaagcccctaagctcctgatctatggcacatccactttgcaaagtggggtcccatcgaggttcagcggcactggatctgggacagaattcattctcacaatcagcagcctgcagcctgaagattttgcaacttattactgtcaacaacttgatagttaccctgtcaccttcggcggcgggacccgggtggagatcaaac

SEQ ID NO: 91 - DNA, 334 bp, synthetic construct
  source 1..334: mol_type = other DNA
cagtctgtcctgactcagcccccctcagcgtctgggacccccgggcagggggtcaccatctcttgttctggaagcaactccaacatcggaactaatgctgtcgactggtaccagcaattcccaggaacggcccccagactcctcatttttaatgataatcagcggccctcaggggtccctgatcgattctctggctccaggtctggcacctcagcctccctggccatcagtggactccagtctgaggatgaggctgtatattattgtgcaacatgggatgacagcctcaatggtcccgtggttttcggcggagggaccaagctgaccgtcctac

SEQ ID NO: 92 - DNA, 322 bp, synthetic construct
  source 1..322: mol_type = other DNA
gaaattgtgttgacacagtctccagccaccctgtctttgtctccaggggaaagagccaccctctcctgcagggccagtcagagtgttagtacctacttagcctggtaccaacagaaacctggccaggctcccaggctcctcatgtatgatgcatccaacagggccactggcatcccagccaggttcagtggcagtgggtctgggacagacttcactctcaccatcagcagcctagagtctgaagattttgcggtttattactgtcagcaacgtagcatccggccgtggacgttcggccaagggaccaaggtggagatcaaac

SEQ ID NO: 93 - DNA, 322 bp, synthetic construct; identical to SEQ ID NO: 92
  source 1..322: mol_type = other DNA

SEQ ID NO: 94 - DNA, 331 bp, synthetic construct
  source 1..331: mol_type = other DNA
gaaattgtgttgacgcagtctccaggcaccctgtctttgtctccaggggaaagagccaccctctcctgcagggccagtcagagtgttggcagcagctacttagcctggtaccaacagaaacctggccagactcccaggctcctcatctatggtgcatccaatagggccactggcatccccgacaggttcagtggcagtgggtctgggacagacttcactctcaccatcagcgggctggagcctgaagattttgcagtgtattactgtcagcactatggtcggtcacctctctcgtcgacattcggccaggggaccaaggtggaaatcaaac

SEQ ID NO: 95 - DNA, 322 bp, synthetic construct
  source 1..322: mol_type = other DNA
gaaattgtgttgacacagtctccagccaccctgtctttgtctccaggggaaagagccaccctctcctgcagggccagtcagagtgttagtacctacttagcctggtaccaacagaaacctggccaggctcccaggctcctcatgtatgatgcatccaacagggccactggcatcccagccaggttcagtggcagtgggtctgggacagacttcactctcaccatcagcagcctagagtctgaagattttgcggtttattactgtcagcaacgtagcatccggccgtggacgttcggccaagggaccaaggtggaaatcaaac

SEQ ID NO: 96 - DNA, 334 bp, synthetic construct
  source 1..334: mol_type = other DNA
gatattgtgatgactcagtctccactctccctgcccgtcacccctggagagccggcctccatctcctgcaggtctagtcagagcctccgacatagtgatggaaacaactatttggattggtacctgcagaagccagggcagtctccacagctcctgatctatttgggttctaatcgggcctccggggtccctgacaggttcagtggcagtggatcaggctcagattttacactgaaaatcagcagagtggaggctgaggatgttggggtttattactgcatgcaagctctacaaactcccacttttggccaggggaccaaggtggagatcaaac

SEQ ID NO: 97 - DNA, 325 bp, synthetic construct
  source 1..325: mol_type = other DNA
tcctatgagctgactcagccaccctcagtgtcagtggccccaggaaagacggccaggattacctgtgggggaaacaacattggaagtaaaagtgtgcactggtaccagcagaagccaggccaggcccctgtgctggtcatctattatgatagcgaccggccctcagggatccctgagcgattctctggctccaactctgggaacacggccaccctgaccatcagcagggtcgaagccggggatgaggccgactattactgtcaggtgtgggatagtagtagtgatcatccggtgttcggcggagggaccaagctgaccgtcctag

SEQ ID NO: 98 - protein, 22 aa, synthetic construct
CARSKGYSNDYLKYXXYGMDVW

SEQ ID NO: 99 - protein, 12 aa, synthetic construct
CQHFDNVPMYTF

SEQ ID NO: 100 - protein, 15 aa, synthetic construct
CARARVNGVFESDYW

SEQ ID NO: 101 - protein, 11 aa, synthetic construct [sequence omitted]

SEQ ID NO: 102 - protein, 25 aa, synthetic construct
CVRESAVISSPSWGLYDTAYHFDPW

SEQ ID NO: 103 - protein, 14 aa, synthetic construct
CATWDDSLNGPVVF

SEQ ID NO: 104 - protein, 17 aa, synthetic construct
CARESWEGAYLKYFDFW

SEQ ID NO: 105 - protein, 11 aa, synthetic construct [sequence omitted]

SEQ ID NO: 106 - protein, 28 aa, synthetic construct
CVRDQYDFWTSYYTGVEGRHYYYGLDVW

SEQ ID NO: 107 - protein, 13 aa, synthetic construct
CQHYGRSPLSSTF

SEQ ID NO: 108 - protein, 17 aa, synthetic construct
CARGLSRYTRELLFHYW

SEQ ID NO: 109 - protein, 13 aa, synthetic construct [sequence omitted]

SEQ ID NO: 110 - protein, 16 aa, synthetic construct
CAKFFRSDGVFHFDYW

SEQ ID NO: 111 - protein, 10 aa, synthetic construct [sequence omitted]

SEQ ID NO: 112 - protein, 15 aa, synthetic construct
CVKGRYTIADYFEHW

SEQ ID NO: 113 - protein, 11 aa, synthetic construct
CQQNYDLPFTF

SEQ ID NO: 114 - DNA, 420 bp, synthetic construct
  source 1..420: mol_type = other DNA
atgaaacatctctggttctttctcctgttggtagcagcgccccgctgggtgttgagccaggtccggctggtggaatcagggggcggggtggtacagcctgggggtagcctccgactctcctgtgaaacttccggcttttcactttcctcctttgggatgcattgggtccggcaggcccccggaaaagggctcgagtgggtagcctttattagagacgatgggcgtaacactaactacgccgactccgtcaagggccgctttacaatcagccgcgatatatctaagaacactctctatctcgagatgaaatcactgcgcgttgaggacacggccacatattactgcgccaagaagggcgccagtaatcactacttttacatcgacgtttggggtcggggcactactgttaccgttagctct

SEQ ID NO: 115 - DNA, 384 bp, synthetic construct
  source 1..384: mol_type = other DNA
atgaaacatctgtggtttttccttctcctcgtggccgctccccgctgggtgctttcagaggccgtgctcacacagagccccggaaccttgtccctgtccccaggcgagagagcaactctttcctgtagagccagtcagtcagtgtctagctcattcctggcgtggtaccagcacaaacctgggcaggctcctcgccttttgatatatggcgcttcaaacagggctaccggaatcccggaccggttttcaggatccgggtctggtgcagattttacactgaccataacaagtttggagccagaggactttgctgtgtactattgtcatcaatatggaagtgccttgaagtggactttcggtcagggcacaaaagtagagattaaa

SEQ ID NO: 116 - DNA, 423 bp, synthetic construct
  source 1..423: mol_type = other DNA
atgaagcacctgtggtttttccttcttcttgtggcggcacctcgttgggtcctcagtcaggtgacactgaaggagagcgggcctgccttggttaagccaacgcagacactcaccttgacatgcacgttctcaggcttcagcctctccacctctgggatgagggtgtcctggatccgacaaccccccgggaaagccctcgaatggctggccagaatcgattgggacgacgataagtattactccacttccctgaagacgaggctgaccattagtaaggacacctccaataaccaggttgtgttaacaatgaccaatatggatcctgcagatacagcgacctattactgcgctcggtcttatagcaactcctggtattcatggtttgatccatggggccaggggacactggtgacagtgagttcc

SEQ ID NO: 117 - DNA, 378 bp, synthetic construct
  source 1..378: mol_type = other DNA
atgaagcacctctggtttttcttgctccttgtcgctgcccctcggtgggtgctgagtgatatccaaatgacccagagccccagttccctttcagccagcgccggggatagagtgaccattacttgccgggccagtcagtctatcagcagcttcctcaattggtaccagcaaaaacaaggcaaggcaccgaaccttctgatctatgcagcttccagcctgcagtcaggcgtgcccagccggttctccgggagtgggagcggtactgattttaccttaactatcagttcccttcagcccgaggatttcgcaacgtattattgtcagcagtcttactccatcccctatacctttggtcagggaaccaagctggagattaaa

SEQ ID NO: 118 - DNA, 402 bp, synthetic construct
  source 1..402: mol_type = other DNA
atgaagcacctgtggttctttctcctcctggtggccgccccgcggtgggtgctgtccgaggtgcaattggtacaaagtggcgctgaggttaagaaaccaggagagtccctgaaaatatcttgcaagggctccggatacatttttaccacctattggatcggctgggtgcgccaaatgccaggtaaaggtctggagtggatgggcataatttacccgggggactccgacatccggtactccccttctttcgagggacaagtgacaattagcgccgataaatcaattagcaccgcctatcttcagtggcccagtctcaaggcttccgatactgccatgtattattgtgcgatcctggacggaatgggcgtgtggggacaaggaactacagtgaccgtctcaagt

SEQ ID NO: 119 - DNA, 402 bp, synthetic construct
  source 1..402: mol_type = other DNA
atgaagcatctctggttctttctgcttctcgtcgcagctcctaggtgggtactgagtcaggcggtgttgacgcagccctcctctttatctgcgagcccaggcgctagcgcttcactgacctgtactttgcgctctgggataaacgtggccatatatatgattcattggtatcagcagaagccaggctcccccccccagtatctgttaaggtataaaagcgattctgataaacaacagggatcaggcgttccgtcacggtttagcggtagtaaagacgctagtgccaacgctgggattcttctgatctccgggctccagagcgaggacgaggctgattactattgtcttatctggcatagtaacgcctgggtttttggtgggggcacgaaactcacagtactg

SEQ ID NO: 120 - DNA, 402 bp, synthetic construct
  source 1..402: mol_type = other DNA
atgaagcacctgtggttttttcttttgctggtggccgctcctagatgggttctgtccgaagtgcagctggttgagagtggtggtggattggttcaaccgggtggaagtctccgtctgagttgcgccgcgtctggattcactttctcaacctattggatgacgtgggtgagacaggcacctggaaaaggcctggaatgggtcgccaatattaaagaggacggttctgaaaagcattatgtcgactccgtgaagggccgcttcactatctcacgcgataacgccaaaaactcactttatctccagatgaattctctgcgggacgaggacacagcagtatattactgtgccagaggtatggccagagacccatggggccagggtacattggtcgccgtgagcagc

SEQ ID NO: 121 - DNA, 378 bp, synthetic construct
  source 1..378: mol_type = other DNA
atgaagcatttgtggttctttcttcttctcgtggctgcgcctcgctgggtcctctctgacatccagatgactcaaagcccctccactcttagcgctagcgtcggcgacaaagtcacgatcacatgcagagcatctcagaccattggctcctggttagcgtggtatcaacaaaagcccggcaaggcccccaacctgctgatttatatggctagcacactggagtccggtgtcccatccagattttccggatctggaagcggcacagagttcactcttaccatcagctcccttcaaccagatgactttgctacctactattgccatcaatacgactcctatccctatacatttggacgggggacaaaattggaaataaaa

SEQ ID NO: 122 - DNA, 414 bp, synthetic construct
  source 1..414: mol_type = other DNA
atgaaacacctgtggttcttccttctcttagtagcggcccctcggtgggtcctgtcagaggtgcaattggtgcagagtggggcggaggtcaaaaagcccggggaatctctcaagattagctgcaagggatccgggtatagctttacttccaactggatcgcatgggttagacagatgcctggcaaagggctggagtggatgggcgtgatttatccaggggactctgacacaaggtacaggcccagtttccagggacaggtgactatcagcgccgacaagtccatcagcactgcctatctgcagtggagtagtctgaaggccagtgacaccgccatgtattattgcgcacgcagagggagtagaggcttcgactactggggccaggggaccctggtgacagtaaccgtgtcctct

SEQ ID NO: 123 - DNA, 375 bp, synthetic construct
  source 1..375: mol_type = other DNA
atgaaacacctgtggtttttcctgcttctcgttgccgctccacggtgggttcttagctacgagctgtctcagccgccatccgtatctgttagcccaggtcagaccgcctctattacctgtagcgccgacaagctcggcaacgagaacgtatgttggtatcagcaaaaacctggccagtcccccgtggttgtaatttatcaaaataacaagagacccagcggaatccccgagcgtttctccggctccaagagcggaaacactgcaaccttaacgatcagtgggacccaagccatggatgaagcagattactactgccagtcctgggactctagcaccgccgaggtcttcggaggaggaacgaagctcacagtcctc

SEQ ID NO: 124 - DNA, 402 bp, synthetic construct
  source 1..402: mol_type = other DNA
atgagagcctggatctttttcctgctgtgcctggctgggcgcgccctggccgaggtccaacttgttcagagcggtgctgaggtgaaaaaacccggcgaatcactgaagatatcctgcaaggggagtggttactccttcactagtaactggatcgggtgggtaaggcagatgcccggcaaggggctggaatggatggggatcatttatcccggcgattctgatacacgatataacccgccctttcagggccaggtcactatgagtgcagataaaagcatttcagcagcctttctgcagtggtctagcctcaaagcttccgatactgctatgtactattgtgcgaggcaatccagcaggggtttcgattactgggggcagggcactcttgttatcgtcagctcc

SEQ ID NO: 125 - DNA, 387 bp, synthetic construct
  source 1..387: mol_type = other DNA
atgagagcctggatctttttcctgctgtgcctggctgggcgcgccctggcccagtctgtgctcactcagccccctagcgtcagtggcgccccgggccagcgtgttaccattagctgtaccgggtcatcctccaatataggggcgggctataacgtgcattggtatcagcagctcccgggaacagcacctaaactgcttatatatggtaataataataggccatccggcgtgcccgataggtttagcggatcaaagtcagggactagcgcaagcctggcaattactggacttcaagccgaggatgaagccgattactactgccagtcctacgatacatccctgtccgggtccggcgtttttggtgggggcacaaagctcaccgtcctg

SEQ ID NO: 126 - DNA, 426 bp, synthetic construct
  source 1..426: mol_type = other DNA
atgagagcctggatctttttcctgctgtgcctggctgggcgcgccctggcccaggttcacctcgtggagtctggaggcggggtggtccagcccggccggtctctccggctgagctgcgttgcctctgggtttacattctccacatacggcatgcactgggtgaggcaggctccggggaaaggcttggagtgggtcgctatcattagcgatgatggatccaatgagtcctacgctgattctgccaagggtaggtttactatatctagggacaactccaggaataccgtccatctgcagatgaattctctgaagactgaggacacggccgtgtattattgtggcaacggatatagaaactattggaatgacattcgagggggccccctgtataagtggggtcagggcaccatggtgactgtcagttcc

SEQ ID NO: 127 - DNA, 381 bp, synthetic construct
  source 1..381: mol_type = other DNA
atgagagcctggatctttttcctgctgtgcctggctgggcgcgccctggccagctacgtcttaacccagcctccttctgtaagtgtcgcccccggacagaccgctacaatcacgtgcgggggaaatagggtcggttccaagtcagtccactggtatcagcagaagcctggacaggcaccggtccttgtggtgtacgacgacagcgaccggccctccggaatccccgagaggttctctggttctaactttgggaatacagctacactgactatcactggcgtcgaggccggtgacgaagccgattactactgccaagtatgggataacaatggtgtgcatccccacgtaatttttggcggtggcgcgaaactgaccgttctg

SEQ ID NO: 128 - DNA, 426 bp, synthetic construct
  source 1..426: mol_type = other DNA
atgagagcctggatctttttcctgctgtgcctggctgggcgcgccctggcccaggtgcagctccaacaatggggcgcaggacttctcacacccagcgaaacgctctcactgacctgcgccgtgtttggcggttcattcagcgattactattggtcctggatacgtcaatcaccaggcaaaggactggagtggatcggcgaaatcaatcactctggggataaaacatacaatccatcactgcaatctcgcatcgcgattagcgtagacacttcaaagaagcagtttagtctgaaacttaattccgtcacagctgccgacacagcggtctatttctgtgcccgccaaggccgggttacaacctttggcgtgttgatcgaggggttctttgatctgtggggcagagggacgcttgtaacagtgagtagc

SEQ ID NO: 129 - DNA, 372 bp, synthetic construct
  source 1..372: mol_type = other DNA
atgagagcctggatctttttcctgctgtgcctggctgggcgcgccctggccgatatacagatgacccagtctccctctagtttgagtgcttctgtgggagaccgcgtaacgattatatgccgggcttcccagaacatcgacacgtacctgaattggtatcagcaaaagcctgggaaggcacctaagttcctgatctacgctgccagtactctccaaaatggagttccgtccaggttctctggatccggcagcggcactgattttactttaactattaacagtctgcagcctgaggatttcgctacatatttctgtcaacagagtcactccacacccctgacgtttgggggcggaacccgcgtggaaattaag

SEQ ID NO: 130 - DNA, 360 bp, synthetic construct
  source 1..360: mol_type = other DNA
gaggtgcacctcctggagtctgggggaggcctggtccagcctggggggtcccgcagaatctcctgtgcagcctctggattctcttttggcagctatgccatgagctgggtccgccaggctccagggaaggggctggagtgggtctcgactataactagtagtggtgaaagcacagactacgcagactccgtgaagggccggttcatcatctccagagacaacgccaagaacacgctggatctcctaatgaacagcctgagagccggggacacggccgtttactactgtgcgaaggggggggcagtagtactgagcgctatggacgtctggggccaagggactacggtcaccgtctcctca

SEQ ID NO: 131 - DNA, 321 bp, synthetic construct
  source 1..321: mol_type = other DNA
gacatccagttgacccagtctccatcttccctgtctgcatctgtaggagacagggtcaccatcacttgccaggcgagtcacgacattagtaactatctaaattggtatcagcagaaaccagggaaagcccctaagctcctgatctacgatgcatccaatttggaaacaggggtcccaccaaggttcagtggaggtggatctgggacacattttagtctcaccatcagcagcctgcagcctgaagacattgcaacatattactgtcaacaggatgatagtctcccattaactttcggccctgggaccaaggtggaaatcaaa

SEQ ID NO: 132 - DNA, 354 bp, synthetic construct
  source 1..354: mol_type = other DNA
gaggtgcagctgttggagtcggggggaggcttggtacagcctggggggtccctgagactctcctgtgcagcctctggattcacctttagcagccatgccatgagctgggtccgccaggctccagggaaggggctggagtgggtctcaactattggtagtagtggcattagtacatactacacagactccgtgaagggccgcttcaccatctccagagacaattccaagaacacgctgtttcttcaactgaacagcctgagagccgaggacacggccgtctattactgtgaagtggggggggatcaaatcgcgtcttcctactggggccagggaaccctggtcaccgtctcctca

SEQ ID NO: 133 - DNA, 324 bp, synthetic construct
  source 1..324: mol_type = other DNA
gacatccagatgacccagtctccttccaccctgtctgcatctgttggggatagagtcaccatcacttgccgggccagtgagaatattaatagttggttggcctggtatcagcagaaaccagggaaagcccctaaactcctgatctacaaggcgtctagtttacaaagtggggtcccatcaaggttcagcggcagtagatctgggacagaattcactctcaccatcagtagcctgcagcctgatgattttgcaacttattactgccaacagtataatacttatttctcgtacacttttggccaggggaccaagttggagatcaaa

SEQ ID NO: 134 - protein, 121 aa, synthetic construct
QVRLVESGGGVVQPGGSLRLSCETSGFSLSSFGMHWVRQAPGKGLEWVAFIRDDGRNTNYADSVKGRFTISRDISKNTLYLEMKSLRVEDTATYYCAKKGASNHYFYIDVWGRGTTVTVSS

SEQ ID NO: 135 - protein, 109 aa, synthetic construct
EAVLTQSPGTLSLSPGERATLSCRASQSVSSSFLAWYQHKPGQAPRLLIYGASNRATGIPDRFSGSGSGADFTLTITSLEPEDFAVYYCHQYGSALKWTFGQGTKVEIK

SEQ ID NO: 136 - protein, 122 aa, synthetic construct
QVTLKESGPALVKPTQTLTLTCTFSGFSLSTSGMRVSWIRQPPGKALEWLARIDWDDDKYYSTSLKTRLTISKDTSNNQVVLTMTNMDPADTATYYCARSYSNSWYSWFDPWGQGTLVTVSS

SEQ ID NO: 137 - protein, 107 aa, synthetic construct
DIQMTQSPSSLSASAGDRVTITCRASQSISSFLNWYQQKQGKAPNLLIYAASSLQSGVPSRFSGSGSGTDFTLTISSLQPEDFATYYCQQSYSIPYTFGQGTKLEIK

SEQ ID NO: 138 - protein, 115 aa, synthetic construct
EVQLVQSGAEVKKPGESLKISCKGSGYIFTTYWIGWVRQMPGKGLEWMGIIYPGDSDIRYSPSFEGQVTISADKSISTAYLQWPSLKASDTAMYYCAILDGMGVWGQGTTVTVSS

SEQ ID NO: 139 - protein, 115 aa, synthetic construct
QAVLTQPSSLSASPGASASLTCTLRSGINVAIYMIHWYQQKPGSPPQYLLRYKSDSDKQQGSGVPSRFSGSKDASANAGILLISGLQSEDEADYYCLIWHSNAWVFGGGTKLTVL

SEQ ID NO: 140 - protein, 115 aa, synthetic construct
EVQLVESGGGLVQPGGSLRLSCAASGFTFSTYWMTWVRQAPGKGLEWVANIKEDGSEKHYVDSVKGRFTISRDNAKNSLYLQMNSLRDEDTAVYYCARGMARDPWGQGTLVAVSS

SEQ ID NO: 141 - protein, 107 aa, synthetic construct
DIQMTQSPSTLSASVGDKVTITCRASQTIGSWLAWYQQKPGKAPNLLIYMASTLESGVPSRFSGSGSGTEFTLTISSLQPDDFATYYCHQYDSYPYTFGRGTKLEIK

SEQ ID NO: 142 - protein, 119 aa, synthetic construct
EVQLVQSGAEVKKPGESLKISCKGSGYSFTSNWIAWVRQMPGKGLEWMGVIYPGDSDTRYRPSFQGQVTISADKSISTAYLQWSSLKASDTAMYYCARRGSRGFDYWGQGTLVTVTVSS

SEQ ID NO: 143 - protein, 106 aa, synthetic construct
YELSQPPSVSVSPGQTASITCSADKLGNENVCWYQQKPGQSPVVVIYQNNKRPSGIPERFSGSKSGNTATLTISGTQAMDEADYYCQSWDSSTAEVFGGGTKLTVL

SEQ ID NO: 144 - protein, 117 aa, synthetic construct
EVQLVQSGAEVKKPGESLKISCKGSGYSFTSNWIGWVRQMPGKGLEWMGIIYPGDSDTRYNPPFQGQVTMSADKSISAAFLQWSSLKASDTAMYYCARQSSRGFDYWGQGTLVIVSS

SEQ ID NO: 145 - protein, 112 aa, synthetic construct
QSVLTQPPSVSGAPGQRVTISCTGSSSNIGAGYNVHWYQQLPGTAPKLLIYGNNNRPSGVPDRFSGSKSGTSASLAITGLQAEDEADYYCQSYDTSLSGSGVFGGGTKLTVL

SEQ ID NO: 146 - protein, 125 aa, synthetic construct
QVHLVESGGGVVQPGRSLRLSCVASGFTFSTYGMHWVRQAPGKGLEWVAIISDDGSNESYADSAKGRFTISRDNSRNTVHLQMNSLKTEDTAVYYCGNGYRNYWNDIRGGPLYKWGQGTMVTVSS

SEQ ID NO: 147 - protein, 110 aa, synthetic construct
SYVLTQPPSVSVAPGQTATITCGGNRVGSKSVHWYQQKPGQAPVLVVYDDSDRPSGIPERFSGSNFGNTATLTITGVEAGDEADYYCQVWDNNGVHPHVIFGGGAKLTVL

SEQ ID NO: 148 - protein, 125 aa, synthetic construct
QVQLQQWGAGLLTPSETLSLTCAVFGGSFSDYYWSWIRQSPGKGLEWIGEINHSGDKTYNPSLQSRIAISVDTSKKQFSLKLNSVTAADTAVYFCARQGRVTTFGVLIEGFFDLWGRGTLVTVSS

SEQ ID NO: 149 - protein, 107 aa, synthetic construct
DIQMTQSPSSLSASVGDRVTIICRASQNIDTYLNWYQQKPGKAPKFLIYAASTLQNGVPSRFSGSGSGTDFTLTINSLQPEDFATYFCQQSHSTPLTFGGGTRVEIK

SEQ ID NO: 150 - protein, 120 aa, synthetic construct
EVHLLESGGGLVQPGGSRRISCAASGFSFGSYAMSWVRQAPGKGLEWVSTITSSGESTDYADSVKGRFIISRDNAKNTLDLLMNSLRAGDTAVYYCAKGGAVVLSAMDVWGQGTTVTVSS

SEQ ID NO: 151 - protein, 107 aa, synthetic construct
DIQLTQSPSSLSASVGDRVTITCQASHDISNYLNWYQQKPGKAPKLLIYDASNLETGVPPRFSGGGSGTHFSLTISSLQPEDIATYYCQQDDSLPLTFGPGTKVEIK

SEQ ID NO: 152 - protein, 118 aa, synthetic construct
EVQLLESGGGLVQPGGSLRLSCAASGFTFSSHAMSWVRQAPGKGLEWVSTIGSSGISTYYTDSVKGRFTISRDNSKNTLFLQLNSLRAEDTAVYYCEVGGDQIASSYWGQGTLVTVSS

SEQ ID NO: 153 - protein, 108 aa, synthetic construct
DIQMTQSPSTLSASVGDRVTITCRASENINSWLAWYQQKPGKAPKLLIYKASSLQSGVPSRFSGSRSGTEFTLTISSLQPDDFATYYCQQYNTYFSYTFGQGTKLEIK